(19) United States
(12) Patent Application Publication
(10) Pub. No.: US 2004/0170668 A1
(43) Pub. Date: Sep. 2, 2004

(54) Use of docosahexaenoic acid and arachidonic acid enhancing the growth of preterm infants

(75) Inventors: Deborah A. Schade, Evansville, IN (US); Kimberly L. Merkel, Evansville, IN (US); James W. Hansen, Evansville, IN (US)

Correspondence Address: Robert S.Thomas, P.O. Box 11070, Columbia, SC, 29211-1070, US

(73) Assignee: Bristol-Myers Squibb Company, Princeton, NJ (US)

(21) Appl. No.: 10/713,936

(22) Filed: Nov. 14, 2003

Related U.S. Application Data
(62) Division of application No. 09/381,484, filed on Feb. 28, 2000.
Application No. 09/381,484 is a 371 of international application No. PCT/US98/10566, filed on Mar. 20, 1998.
(60) Provisional application No. 60/042,366, filed on Mar. 27, 1997.

(51) Int. Cl. A61K 47/00; A61K 31/202

(52) U.S. Cl. 424/439; 514/560

(57) ABSTRACT

A method for enhancing the growth of preterm infants involving the administration of certain long chain polyunsaturated fatty acids. It is preferred that the infants are administered an infant formula containing a combination of docosahexaenoic acid and arachidonic acid.

### CROSS-REFERENCE TO RELATED PATENT APPLICATIONS

The present application is a Divisional of U.S. patent application Ser. No. 09/381,484, filed Feb. 19, 2002, which is a Continued Prosecution Application of United States Non-provisional patent application Ser. No. 09/381,484, filed Sep. 21, 1999 under 35 U.S.C. § 371, which is a National Phase Application of International Application No. PCT/US98/10566, filed Mar. 20, 1998, which designated the United States and claims the benefit of U.S. Provisional Application Serial No. 60/042,366, filed Mar. 27, 1997, and claims the priority benefit of each of these applications, each of which is incorporated herein by reference in its entirety, and is related to a commonly assigned and copending application having the title “Use of Docosahexaenoic Acid and Arachidonic Acid Enhancing The Growth of Preterm Infants”, which was filed on the same date as the present application.

### BACKGROUND OF THE INVENTION

(1) Field of the Invention

The present invention concerns enhancing the growth of preterm infants involving administration of infant formula containing a combination of docosahexaenoic and arachidonic acid.

(2) Description of the Related Art

The long chain polyunsaturated fatty acids (LC PUFA) have been shown to be important in infant development. Particularly, arachidonic acid (ARA) and docosahexaenoic acid (DHA) are LC PUFA that are of special interest in infant nutrition because they are found in high concentrations in the brain (Sastry P S, Lipids of nervous tissue: composition and metabolism. Progress Lipid Res 1985;24:69-176) and the retina (Fliesler S J and Anderson R E. Chemistry and metabolism of lipids in the vertebrate retina. Progress Lipid Res 1983;22:79-131). ARA (20:4n-6) and DHA (22:6n-3) are derived from the parent essential fatty acids linoleic acid (18:2n-6) and α-linolenic acid (18:3n-3) through alternate desaturation and elongation and accumulate rapidly in fetal neural tissue during the last months of gestation and the first months of postnatal life (Makrides M, Neuman M A, Byard R W, Simmer K, Gibson R A. Fatty composition of the brain, retina and erythrocytes in breast- and formula-fed infants. Am J Clin Nutr 1994;60:189-94).

Unlike term infants, preterm infants do not fully benefit from the maternal and placental LC PUFA supply during the last trimester of pregnancy. Even though preterm infants are capable of synthesizing both DHA and ARA from their 18 carbon precursors (Carnielli V P, Wattimena D J L, Luijendijk I H T, Boerlage A, Degenhart H J, Sauer P J J. The very low birth weight premature infant is capable of synthesizing arachidonic and docosahexaenoic acids from linoleic and linolenic acids. Pediat Res 1996;40:169-174), it remains unclear whether the rate of synthesis is adequate to meet the optimal needs for central nervous system accretion in the absence of a dietary supply of these fatty acids. Preterm infants are dependent on their own dietary supply of linoleic and α-linolenic acids through either human milk, which also contains small but significant amounts of ARA and DHA or through commercially available artificial formulas, none of which in the United States contain ARA end DHA.

It has been demonstrated in recent studies (Hoffman D R and Uauy R. Essentiality of dietary ω-3 fatty acids for premature infants: Plasma and red blood cell fatty acid composition. Lipids 1992;27:886-95) that the fatty acid composition of red blood cell membrane lipids in infants receiving formulas supplemented with DHA (0.35% of total fatty acids) was similar to human milk-fed infants. In the same study, Birch (Birch D G, Birch E E, Hoffman D R Uauy R D. Retinal development in very-low-birth-weight infants fed diets differing in Omega-3 fatty acids. Investigation Ophthalmology Visual Science 1992;33:2365-76) found that retinal function improved with the provision of a dietary supply of DHA in very low birth weight infants.

The first year growth of preterm infants fed standard formula compared to marine oil LC PUFA supplemented formula was studied by Carlson et al. (Carlson S E, Cooke, R J, Werkman S H, Tolley E A. First year growth of preterm infants fed standard compared to marine oil n-3 supplemented formula Lipids 1992:27:901-907). The experimental formulas provided 0.2% of total fatty acids as DHA and also provided 0.3% as EPA (20:5n-3). This EPA concentration is higher than found in human milk while the DHA level is similar to human milk. Beginning at 40 weeks from conception, marine oil supplemented infants compared to controls had significantly lower weight, length, and head circumference. From this study, Carlson (Carlson S E, Werkman S H, Peeles J M, Cooke R J, Tolley E A. Arachidonic acid status correlates with first year growth in preterm infants. Proc Natl Acad Sci USA 1993;90:1073-77) hypothesized that dietary ARA could improve first year growth of preterm infants, in the context of restoring growth to the level of control formula containing no LC PUFA.

In another study (Montalto, F B, et al., Pediatric Research, Vol 39, page 316A, abstract no. 1878) it was shown that male infants fed marine oil supplemented formula (containing DHA but essentially no ARA) had, by 4 to 6 months, lower head circumference, length, weight and fat free mass than standard formula fed infants. A third study also showed decreased weight at 9 and 12 months corrected age in preterm infants fed marine oil supplemented formula (with LC PUFA) to 2 months corrected age compared with control formula containing no LC PUFA (Carlson S E, et al., Am. J. Clin. Nutr., 63 pp 687-97,1996).

The prior art has demonstrated that infants with altered tissue LC PUFA levels, resulting from a lack of LC PUFA in their diets, may be at risk for neurological problems, may also have reduced scores on cognitive tests, and may have lower retinal development than human milk-fed infants. Worldwide regulatory organizations such as the WHO/FAO Expert Committee on Fats and Oils in Human Nutrition have recommended that LC PUFA be included in preterm infant formula. These recommendations have been made despite the negative effects observed of DHA supplements on growth. There has been no demonstration in the literature that ARA and DHA, particularly when added to infant formula, enhances the growth of infants above that demonstrated by control formulas not containing ARA and DHA.

### SUMMARY OF THE INVENTION

It has unexpectedly been discovered that preterm infants receiving infant formula supplemented with both DHA and ARA demonstrate enhanced growth. The present invention is directed to enhancing the growth of preterm infants comprising administering to said infants a growth enhancing amount of DHA and ARA.

### DETAILED DESCRIPTION OF THE INVENTION

As reported in a review of preterm infant growth by Carlson, S E, (The Jml of Pediatrics, vol 125, pp 533-8, 1994) “After adjusting for postconceptional age, preterm infants show a decline (rather than a catch-up) in the normalized weight from approximately 2 to 4 months past expected term.”

Several prior art studies have documented the value of administering DHA to infants. However, when DHA, either as the primary LC PUFA or combined with EPA, is administered to preterm infants, said infants suffer from decreased growth. It has been suggested that ARA may be beneficial to growth; however, heretofore the growth effects of administering both DHA and ARA to preterm infants have been unknown. It has been surprisingly discovered that administering the combination of ARA and DHA results in enhanced growth of infants relative to infants fed DHA alone. It has also been discovered that preterm infants administered an infant formula containing ARA and DHA exhibit enhanced growth relative to preterm infants fed control formula without DHA and ARA, such as those formulas currently used in modem nurseries. It has further been discovered that practice of the method of the invention results in growth of preterm infants catching up in an unexpected short time to a reference group of normal term breast fed infants.

The time to achieve growth similar or equivalent to normal term breast fed infants by practice of the method of the invention is less than 9 months corrected age; preferably less than 6 months corrected age, more preferably less than 4 months corrected age, even more preferably less than 2 months corrected age, and most preferably no greater than term corrected age.

The method of the invention requires a combination of DHA and ARA. The weight ratio weight of ARA:DHA can be about 1:2 to about 5:1, preferably about 1:1 to about 3:1, and more preferably about 2:1.

In the method of the invention the combination of DHA and ARA is preferably administered as part of an infant formula. The infant formula for use in the present invention is preferably nutritionally complete and typically contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, soybean oil, palmolein, coconut oil, medium chain triglyceride oil, high oleic sunflower oil, high oleic safflower oil, and the like. Conveniently, commercially available infant formula can be used. For example, Enfamil®, Enfamil® Premature Formula, Enfamil® with Iron, Lactofree®, Nutramigen®, Pregestimil®, ProSobee® (available from Mead Johnson & Company, Evansville, Ind., U.S.A.), Similac®, Isomil®, Alimentum®, Neocare®, and Similac® Special Care (available from Ross Laboratories, Columbus, Ohio, U.S.A.), may be supplemented with suitable levels of ARA and DHA at the proper ratios and used in practice of the method of the invention.

The form of administration of the DHA and ARA in the method of the invention is not critical, as long as a growth enhancing amount is administered. Most conveniently, the DHA and ARA are supplemented into infant formula which is then fed to the infants. Alternatively, the DHA and ARA can be administered as a supplement not integral to the formula feeding, for example, as oil drops, sachets, in combination with other nutrient supplements such as vitamins, and the like.

The growth enhancing amount of DHA is typically about 2.5 mg/kg of body weight/day to about 60 mg/kg of body weight/day, preferably about 6 mg/kg of body weight/day to about 40 mg/kg of body weight/day, more preferably about 12 mg/kg body weight/day to about 30 mg/kg body weight/day, and even more preferably about 18 mg/kg of body weight/day to about 24 mg/kg of body weight/day.

The growth enhancing amount of ARA is typically about 5 mg/kg of body weight/day to about 120 mg/kg of body weight/day, preferably about 12 mg/kg of body weight/day to about 80 mg/kg of body weight/day, more preferably about 24 mg/kg body weight/day to about 60 mg/kg body weight/day, and even more preferably about 36 mg/kg of body weight/day to about 48 mg/kg body weight/day.

The amount of DHA in infant formulas for use in the present invention typically varies from about 2 mg/100 kilocalories (kcal) to about 50 mg/100 kcal, preferably about 5 mg/100 kcal to about 33 mg/100 kcal, more preferably about 10 mg/100 kcal to about 25 mg/100 kcal, and even more preferably about 15 mg/100 kcal to about 20 mg/100 kcal.

The amount of ARA in infant formula for use in the present invention typically varies from about 4 mg/100 kcal to about 100 mg/100 kcal, preferably about 10 mg/100 kcal to about 67 mg/100 kcal, more preferably about 20 mg/100 kcal to about 50 mg/100 kcal, and even more preferably about 30 mg/100 kcal to about 40 mg/100 kcal.

The infant formula supplemented with oils containing DHA and ARA for use in the present invention can be made using standard techniques known in the art. For example, replacing an equivalent amount of an oil normally present, e.g., high oleic sunflower oil.

The source of the ARA and DHA can be any source known in the art such as fish oil, single cell oil, egg yolk lipid, brain lipid, and the like. The DHA and ARA can be in natural form, provided that the remainder of the LC PUFA source does not result in any substantial deleterious effect on the infant. Alternatively, the DHA and ARA can be used in refined form. It is preferred that the LC PUFA used in the invention contain little or no EPA. For example, it is preferred that the infant formulas used herein contain less than about 20 mg/100 kcal EPA; preferably less than about 10 mg/100 kcal EPA; more preferably less than about 5 mg/100 kcal EPA; and most preferably substantially no EPA.

Preferred sources of DHA and ARA are single cell oils as taught in U.S. Pat. Nos. 5,374,657, 5,550,156, and 5,397,591, the disclosures of which are incorporated herein by reference in their entirety.

The following examples are to illustrate the invention but should not be interpreted as a limitation thereon.

### EXAMPLES

### Clinical Study Design

1. Introduction

This study is a double-blind, randomized, controlled parallel design, prospective trial of premature infant formulas containing microalgae and fungi-derived oils which contain a part of their constituents arachidonic acid and docosahexaenoic acid. Formula feeding subjects will be randomized into one of 3 feeding groups:

premature formula plus DHA (about 0.13% of energy) and ARA (about 0.26% of energy)

premature formula plus DHA (about 0.13% of energy)

premature formula WITHOUT DHA and ARA

The products have the same nutrient composition (see Appendix A) and differ only in the level of DHA and ARA. The products will be blinded. The present order of formula has no relationship to randomization.

Normal, term, breast fed infants will be enrolled to provide a normal visual acuity reference.

Fifty evaluable subjects will be completed in each group. Premature infants will remain on study formulas after reaching 90 kcal/kg/d for a minimum of 28 days or until hospital discharge whichever is longer. After 28 days or discharge, whichever is longer, all premature infants will receive Enfamil or Enfalac with Iron. If medically indicated, ProSobee, Lactofree, Alactamil, Nutramigen, or Pregestimil may be used in place of Enfamil or Enfalac with Iron. Term infants will receive at least 85% of their nutrition from breast milk. Primary measures of effectiveness will include visual acuity and red blood cell membrane fatty acid profiles (i.e. DHA and ARA levels). The measure of safety will be growth and adverse experience reports.

2. Subjects

2.1 Source and Characterization of Study Group

Acceptable preterm subjects will be relatively healthy premature infants taking preterm formula. Anticipated hospitalization should be sufficient to allow for 28 days of enteral intake ≧90 kcal/kg/d and ≧85% study formula intake. All races and both sexes will be eligible for the study.

2.2. Inclusion Criteria

Preterm infants:

Birth weight≧900 g

Formula feeding at time of study enrollment

Anticipate enteral intake of ≧90 kcal/kg/day for ≧28 days before discharge home

Informed consent obtained

Term Infants:

38 to 42 weeks gestation

Committed to breast feeding

Informed Consent obtained

2.3 Exclusion Criteria

Preterm infants:

≧1500 g at birth

Preterm and Term Infants:

History of underlying disease or congenital malformation which in the opinion of the investigator is likely to interfere with the evaluation of the subject

More than 24 days between birth and full oral feeds (≧90 kcal/kg/d)

Small (<10th percentile) for gestational age at birth (SGA)

Necrotizing enterocolitis as diagnosed by the physician

Other gastrointestinal disease

Impaired visual or ocular status at birth

2.4 Concomitant Medications, Hospitalizations, Illnesses

No medication which may affect FPL response may be used within 3 days of measurement.

No evidence of viral of bacterial infection during FPL testing.

No medications known to affect lipid metabolism (e.g., heparin at therapeutic levels)

3. Study Product Information

3.1 Formulations

Nutrient composition is included as Appendix A.

4. Study Procedures

4.2.1 Enrollment

Enrollment will take place over a 6 month period. Ideally, sufficient subjects will be enrolled so that 10 subjects in each group complete the study at each site for the multi-center trial. A total of 50 infants per formula group will complete this trial.

4.2.2 Schedule of Events (See Flow Chart, Section 8.4)

4.2.2.1 Recruitment

Mothers of eligible, healthy, preterm formula fed infants and term, breastfed infants will be contacted, the study explained to them, and if they are agreeable, written informed consent obtained.

Term infants may be enrolled anytime from birth until or during the 48 week visit.

4.2.2.2 Randomization

Recruited formula fed subjects will be randomized into study groups. Randomization can occur anytime after enteral feeds reach 50 kcal/kg/day until commencement of full enteral feeds (i.e., ≧90 kcal/kg/day).

4.2.2.3 Feeding

All premature infants will receive their assigned study formula after informed consent has been granted and enteral feeds are at least 50 kcal/kg/day. The infant will remain on study formula 28 days after reaching 90 kcal/kg/d or until hospital discharge, whichever is longer. Oral feeding amount, strength and rate will advance as appropriate for the clinical management of the infant.

All parents will be instructed not to feed solid foods during the study. The parents will be instructed that the study formula or breast milk is to serve as the sole source of food from enrollment to study end.

4.2.2.4 Baseline Data Collection

The following data will be collected by the Investigator at the time of enrollment and randomization on the case report forms:

Informed consent of parent obtained.

Post conceptual age.

That the subject is a premature infant, with Birth weight ≧900 gm and ≧1500 gm or a normal term infant between 38 and 42 weeks gestational age.

That the preterm subject is receiving infant formula or term infant is committed to breast feeding.

Anticipated preterm infant enteral intake of ≧90 kcal/kg/day for ≧28 days prior to discharge home.

That the subject has no history of underlying disease, inborn error of metabolism, or congenital malformation which in the opinion of the Investigator is likely to interfere with the evaluation of the study formulas.

That the subject is not small (<10th percentile) for gestational age at birth.

That the subject does not have necrotizing enterocolitis as diagnosed by a physician.

That the subject does not have a gastrointestinal disease.

No more than 24 days between birth and full enteral feeds (i.e., ≧90 kcal/kg/day).

That the subject did not have impaired visual or ocular status at birth.

Birth date, sex, race.

Birth weight, length and head circumference

4.2.2.5 Investigator Periodic Data Collection

“During hospitalization, preterm subjects will have their weight recorded daily while they are receiving study formula. Length and head circumference will be recorded weekly, along with an additional weight measurement. For a given subject, the same scale should be used for the weekly weight measurement.”

“Weight, length, and head circumference will also be recorded at the 40, 48, and 57 week post conceptual age visit (preterm) and 56 and 119 days of age visit (term).”

4.2.2.6 Blood Draw

When preterm infant enrolls in the study and again at termination of study formula (i.e., hospital discharge or 28 days after reaching 90 kcal/kg/d of study product), the Investigator will ascertain that the infant is essentially solely formula fed. If this criteria is met, 1.2 ml/blood will be drawn for blood lipids. The sample will be processed as described in Appendix B.

An attempt will also be made to draw a similar blood sample at the 48 weeks PCA visit when visual acuity is measured in both term and preterm infants.

4.2.2.7 Visual Acuity by Forced Choice Preferential Looking (FPL) at 48 and 57 Weeks±4 Days Post-Conceptual Age

When the infant is 48 and 57 weeks±4 days post-conceptual age, trained persons at each study site will follow the Teller Acuity Card Procedure for the measurement of visual acuity of all study subjects. It is essential that only persons who are trained in the FPL procedure for determining visual acuity do the testing. If necessary, training of responsible persons and documentation of completion of successful training will be done at Children's Hospital Medical Center Ophthalmology Department in Seattle, Wash., according to the procedure attached as Appendix C.

If the infant cannot complete the procedure at 48 or 57 weeks±4 days postconceptual age (i.e., too fussy, too sleepy, too inattentive) the test should be repeated within 7 days.

4.2.2.8 Interim Evaluation

At preterm infant hospital discharge or 28 days after reaching 90 kcal/kg/d of study formula feeding, whichever is longer, the Investigator will fill out an “Interim Evaluation” form. After reviewing the subject's records and discussion with the parents and staff, the Investigator will indicate:

Whether or not the subject completed at least 28 days of study formula intake ≧90 kcal/kg/d and both blood samples obtained

If the study was not completed, and reason

Whether or not the subject received steroids (glucorticoids)

Investigator's evaluation of the study formula

The first and last dates study material was taken will be recorded.

4.2.2.9 Final Evaluation

At the final study visit (57 weeks postconceptual age) or earlier if the subject drops out, the Investigator will fill out a “Final Evaluation” Case Report Form. After reviewing the subject's records and discussion with the parents, the Investigator will indicate whether the subject:

(1) Completed feeding regiment and all study parameters (i.e., anthropometrics and visual acuity measured).

(2) Did not complete feeding regimen.

(3) Not completed and reason.

4.3 Clinical Observations

4.3.1 Physical Examinations

Subjects will have weight, length and head circumferences recorded at birth, weekly while hospitalized, then at 40, 48, and 57 weeks±4 days postconceptual age.

Body weight will be measured using an electronic balance or a double beam balance accurate to 10 g or ½oz with non-detachable weights. During hospitalization, if more than one such balance is employed in the practice, either one balance should be designated the study balance and all study weights will be carried out on that balance for a particular subject, or the balances will be checked and certified to register the same weight throughout the range of weights expected. Outpatient weights will be obtained on a calibrated office scale.

Documentation indicating balance calibration of the outpatient balance carried out within 12 months of study initiation will be supplied to the Sponsor.

Length will be measured with the infant in recumbent position with the help of two examiners and a suitable measuring apparatus. One person holds the subject's head in contact with a fixed vertical headboard and a second person holds the subject's feet, toes pointing directly upward and, also applying gentle traction. The baby is measured from the headboard to the soles of the feet with a non-stretching tape measure.

Head circumference will be measured, employing a flexible, non-stretchable cloth or vinyl tape.

4.3.2 Visual Acuity by Forced Choice Preferential Looking (FPL)

Visual acuity will be determined at 48 and 57 weeks±4 days postconceptual age according to procedures outlined in Appendix C.

4.3.3 Laboratory Tests

Blood will be drawn from preterm infants by heel prick or venipuncture when study formula is begun and terminated. An attempt will be made to draw blood at 48 weeks±4 days PCA from both term and preterm infants. Procedures for handling the blood are described in Appendix B.

4.4 Flow Chart

| | PRETERM | | | | | | TERM | | |
|---|---|---|---|---|---|---|---|---|---|
| | | Enteral | Termination | Visit 1 | Visit 2 | Visit 3 | Visit 1 | Visit 2 | Visit 3 |
| | | Intake >50 | of Study | 40 wks ± 4 d | 48 wks ± 4 d | 57 wks ± 4 d | 40 wks ± 4 d | 48 wks ± 4 d | 57 wks ± 4 d |
| EVENT | Birth | kcal/kg/d | Formula ♯ | PCA | PCA | PCA | PCA | PCA | PCA |
| Randomization | | √ | | | | | | | |
| Study Formula | | √ | | | | | | | |
| Enfamil w/iron | | | √ | √ | √ | √ | | | |
| Human Milk | | | | | | | √ | √ | √ |
| | Physical | | | | | | Physical | | |
| Weight | √ | √* | √ | √ | √ | √ | √ | √ | √ |
| Length | √ | √* | √ | √ | √ | √ | √ | √ | √ |
| Head | √ | √* | √ | √ | √ | √ | √ | √ | √ |
| Circumference | | | | | | | | | |
| Blood Draw | | √ | √ | | √ | | | √ | |
| Visual Acuity | | | | | √ | √ | | √ | √ |
| Test | | | | | | | | | |
| Parental | | | | √ | √ | √ | | √ | √ |
| Concerns | | | | | | | | | |
| Interim | | | √ | | | | | | |
| Assessment | | | | | | | | | |

| | | |
|---|---|---|
| Final | (when the subject discontinues or completes) | (when the subject discontinues |
| Assessment | | or completes) |

Medical problems related to or affecting formula consumption will be recorded when they occur.
*rocorded weekly during hospitalization
♯ At hospital discharge or 28 days of study formula intake (after reaching 90 kcal/kg/d), whichever is later

5. Criteria for Response

Criteria for response will depend upon the following:

Visual Acuity better than the control formula.

Visual Acuity comparable to breastfed term infant.

Red Blood Cell phosphatidyl ethanolamine DHA and ARA weight % greater than formula control group.

Growth as measured by weight achieved at 48 and 57 weeks postconceptual age comparable to formula control group.

6. Statistics

6.1 Randomization

If the subject meets the inclusion and exclusion criteria, randomization to one of three formula groups will take place. The randomization schedule will be provided by Mead Johnson Research Center. A separate randomization schedule will be provided for males and females.

6.2 Sample Size

The primary parameter of interest is visual acuity as measured by the Forced Choice Preferential Looking (FPL). The minimal clinically relevant difference was determined to be 0.5 octave. A consultant in the field of visual acuity estimated the standard deviation to be 0.5 octave. This value was increased to 0.7 octave in case more variability was experienced in this study. Thirty-two subjects per group are needed to attain 80% power when testing at an alpha level of 0.05.

A sample size estimate of 50 per group was determined to achieve α+0.05, β+0.20, for weight of infants receiving study oil being greater than 400 gm below control at 48 weeks postconceptual age or 500 g below control at 57 weeks postconceptual age with a standard deviation of 800 g. It was therefore determined that 50 subjects per group will be used in the study.

6.3 Analytical Plan

Visual acuity data will be recorded in cycles per cm. These values will be converted to cycles per degree using the following formula:

$$cycles \text{/} degree = \frac{38 \times cycles \text{/} cm}{55}$$

A log transformation will be applied to the data prior to analysis. Analysis of variance techniques will be used to assess feeding regimen group differences in visual acuity. If the overall F test for feeding regimen is significant at an alpha level of 0.05, pairwise comparisons will be made at an alpha level of 0.05. If no significant differences are detected, then a post-study power analysis will be performed to demonstrate that the study had adequate power to detect the minimal clinically relevant difference.

Analysis of variance will be used to assess feeding regimen differences in phosphatidyl choline DHA and ARA levels and in phosphatidyl ethanolamine DHA and ARA levels at each time point. If the overall F test is significant at an alpha level of 0.05, then pairwise comparisons will be made at an alpha level of 0.05.

Analysis of variance will be used to assess feeding regiment differences in weight at 48 and 57 weeks postconceptual age. The statistical model will include terms for feeding regimen, study center, sex and all two-way interactions. Non-significant interactions will be removed from the final statistical model. Two one-sided tests will be performed comparing each experimental formula (EC) with the control formula (CF). The hypothesis to be tested is as follows:

H₀=Weight (CF)≦Weight (EF).

The alternative hypothesis is as follows:

H₁=Weight (CF)>Weight (EF).

If H₀ is rejected and the mean weight of the control formula exceeds that of the experimental formula by more than 400 mg at 48 weeks postconceptual age or by 500 g at 57 weeks postconceptual age then the conclusion is that the experimental formula does not exceed that of the experimental formula by more than 400 g at 48 weeks postconceptual age or by 500 mg at 57 weeks postconceptual age then the conclusion is that the experimental formula does provide adequate growth. If H₀ is not rejected then a post-study power analysis will be performed to demonstrate that the study had adequate power to detect the above mentioned clinically relevant differences. If adequate power is achieved then the conclusion is that the experimental formula does provide adequate growth.

Fisher's exact test will be used to compare the proportion of subjects in each group with illness/symptoms of concern during the study. The analysis will be performed for each type of illness/symptom reported, with classification of investigator terms into similar terminology made as necessary.

### Appendix A

### Nutrient Composition of Formulas

All study formulas are 24 kcal/fl oz and are identical in composition to marketed Enfamil Premature Formula except for the study oils employed. These oils are described in the protocol.

| | STUDY FORMULAS | |
|---|---|---|
| NUTRIENT | AMOUNT/100 kcal | ENFAMIL WITH Fe |
| Protein g | 3 | 2.2 |
| Fat, g | 5.1 | 5.6 |
| Carbohydrate, g | 11.1 | 10.3 |
| Vitamin A IU | 1250 | 310 |
| Vitamin D IU | 270 | 63 |
| Vitamin E IU | 6.3 | .2 |
| Vitamin K mcg | 8 | 8 |
| Thiamine, mcg | 200 | 78 |
| Riboflavin, mcg | 300 | 150 |
| Vitamin B₆, mcg | 150 | 63 |
| Vitamin B₁₂, mcg | 0.25 | 0.23 |
| Niacin, mcg | 4000 | 1250 |
| Folic Acid, mcg | 35 | 15.6 |
| Pantothenate, mcg | 1200 | 470 |
| Biotin, mcg | 4 | 2.3 |
| Vitamin C, mg | 20 | 8.1 |
| Choline, mg | 12 | 15.6 |
| Inositol, mg | 17 | 4.7 |
| Calcium, mg | 165 | 78 |
| Phosphorus, mg | 83 | 53 |
| Magnesium, mg | 6.3 | 7.8 |
| Iron, mg | 1.8 | 0.5 |
| Zinc, mg | 1.5 | 0.78 |
| Manganese, mcg | 6.3 | 15.6 |
| Copper, mcg | 125 | 94 |
| Iodine, mcg | 25 | 6 |
| Sodium mg (mEq) | 39 (1.7) | 27 (1.17) |
| Potassium mg(Meq) | 103 (2.6) | 108 (2.8) |
| Chloride mg (Meq) | 85 (2.4) | 63 (1.77) |

### Final Study Report

Study Design:

This double-blind, parallel-group study (project 3338) was carried out in 16 neonatal centers (study numbers 9698-9709, 9712, 9723, 9743, and 9746) in North America. Three premature infant feedings were compared. Each had the same composition except for the incorporation of fungal and/or micro algal oils up to about 3% of the fat blend to provide the experimental levels of docosahexaenoic acid (DHA) and arachidonic acid (ARA). The control formula (C, Enfamil® Premature Formula) contained no DHA or ARA, the DHA formula (D) contained about 0.15% of energy as DHA (0.34% of fat), and the DHA+ARA formula (DA) contained about 0.14% of energy as DHA (0.33% of fat) and 0.27% of energy as ARA (0.60% of fat). The formulas were fed to 284 randomized infants weighing 846 to 1560 grams at birth for at least 28 days. Upon completion of study formula intake, they were given routine infant formula and followed through 4 months gestationally corrected age. A group of 90 exclusively human milk fed term infants were enrolled and followed to 4 months of age as a reference group (H).

Study Objective and Statistical Analysis:

The primary objective of this study was to establish the safety of feeding D or DA to preterm infants during their initial hospitalization as measured 1) by growth, acceptance and tolerance while consuming the formula for at least 1 month and 2) by close monitoring and observation for a 4 to 5 month follow-up period (4-5 times the treatment period) while consuming unsupplemented routine term infant formula. The primary growth parameter selected was weight with evaluation of the proposition that weight on test formula was greater than or equal to weight on control formula. The one sided statistical test for an adverse effect on growth maximized the power to detect a difference should one be present. A two-sided test was used for all other parameters. A p-value of less than 0.05 was used to establish significance.

Secondary objectives of the study were 1) to evaluate the impact of fatty acid levels in erythrocyte phospholipids at the end of study feeding and 2) to determine if any effect on mean visual acuity greater than half an octave could be demonstrated at 2 and 4 months corrected age.

Results:

Six infants were just outside the weight parameters and five infants just older than the less than 24 days chronological age parameter for enrollment in the study. In each case, judgement by the clinical or medical monitor was made to include them in the study prior to enrollment based on their homogeneity with other study infants in all other particulars, e.g., state of health, type of medical complications, and weight for gestational age. All these infants were included in the analysis of the study results.

The formula groups were comparable at enrollment (See table 1). Post-conceptual age, weight, length, and head circumference at enrollment did not differ among the groups.

All groups experienced comparable final study status (See table 2). Drop outs did not differ among the formula fed groups during hospitalization. There also were no differences in drop outs among the four groups at study completion.

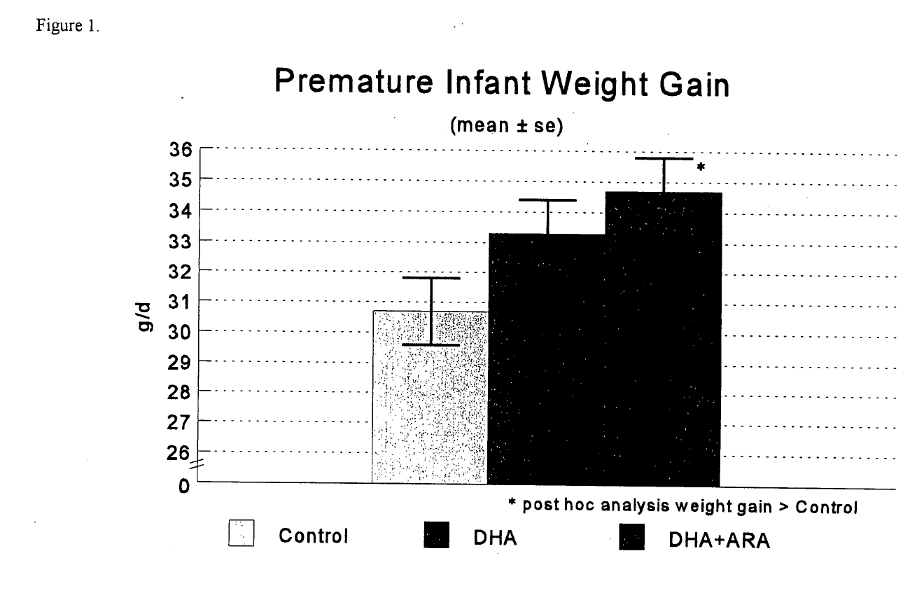

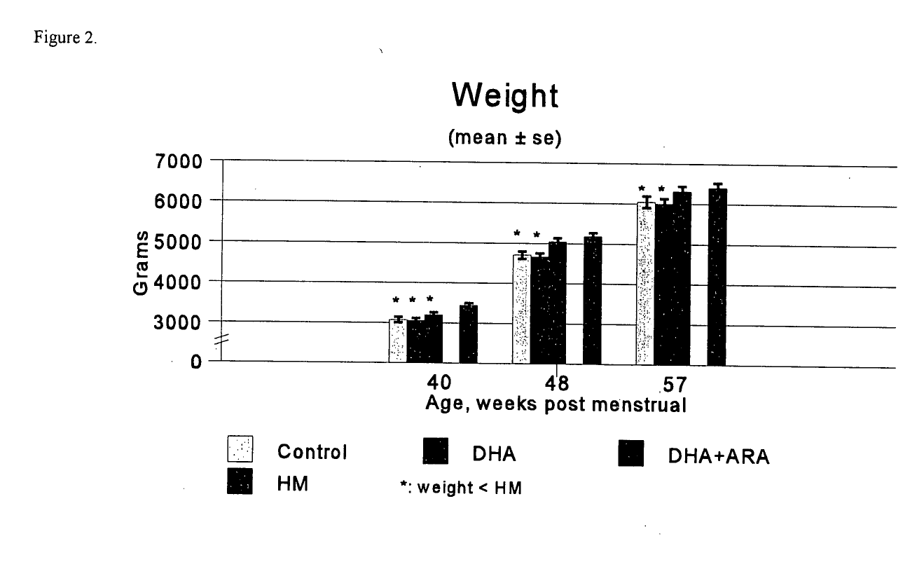

Both formulas D and DA provide adequate growth when compared to formula C (See table 3, FIG. 1, and Appendix 1). Weight gain during hospitalization was no less on D or DA than on C, 33.3, 34.7, and 30.7 g/day, respectively. Furthermore, no less weight was achieved on D or DA than on C at 40, 48, and 57 weeks post-conceptual age (See table 4, FIG. 2, and Appendix 1); statistical power was greater than 0.89 to detect a clinically relevant decrease.

Post-hoc analysis reveals that infants on DA grew faster than infants receiving C and D (See table 5 and FIG. 1). This enhanced growth provided faster “premature infant catch-up” compared to C and D. Weight achieved by the DA group (3198 g) was higher than C (3075 g) and D (3051 g) at 40 weeks post-conceptual age but had not fully caught up to the term birth weight (3438 g) of group H (See table 4 and FIG. 2). This catch up trend continued through 48 to 57 weeks by which time the mean weight of group DA did not differ from group H while groups C and D remained significantly lower.

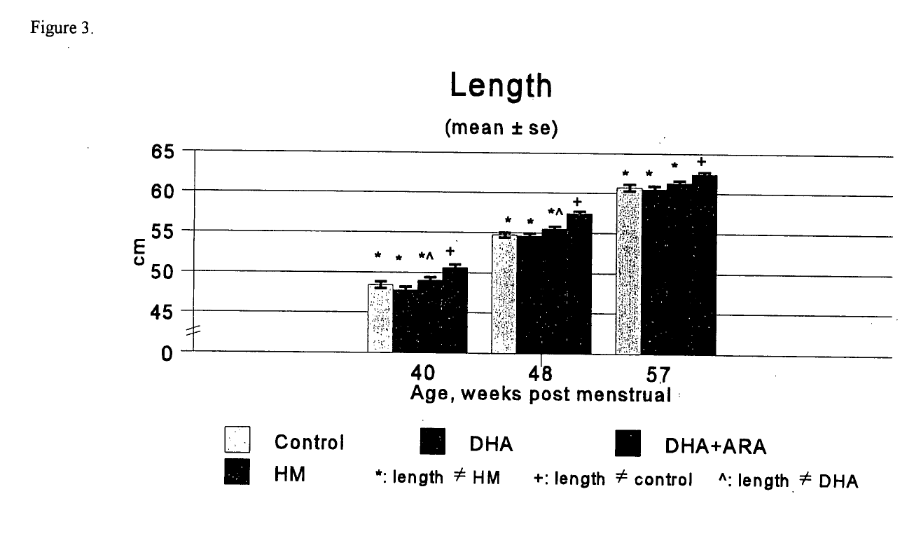

Length was not different among the formula groups either during hospitalization or the follow-up period, although the ordered sequence of mean lengths was the same as for the weights (See table 7 and FIG. 3). This is likely at least partially due to length being a less sensitive parameter of growth than weight. For the same reason, the mean lengths of group H infants were higher than that of all the premature infant groups at 40, 48 and 57 weeks post-conceptual age indicating slower catch up in this parameter.

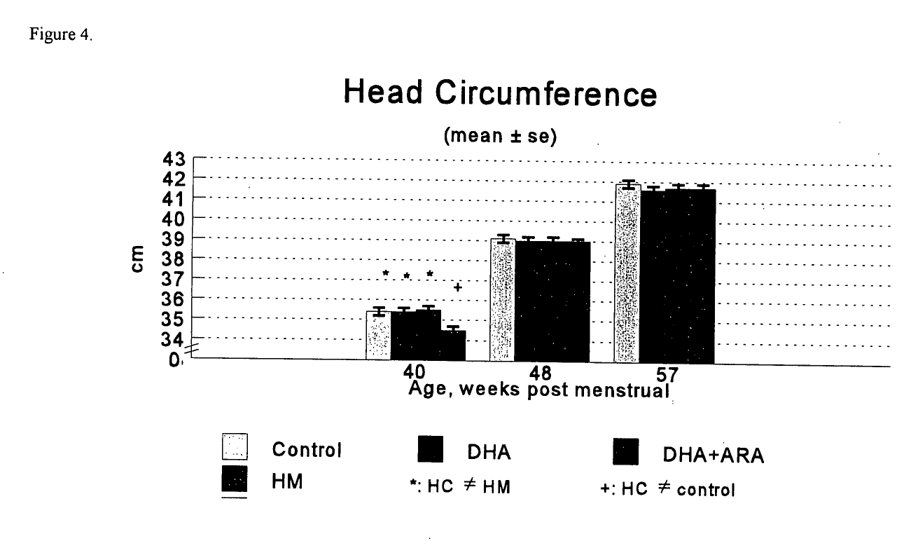

Head circumference is the least sensitive parameter of growth and was not different among any of the four groups at any time measured except at 40 weeks postconceptual age (See table 8 and FIG. 4). At this time, as expected, the birth head circumference of group H was smaller than the formula fed premature infants possibly due to molding of labor and to insufficient time for adjustment to the extrauterine environment.

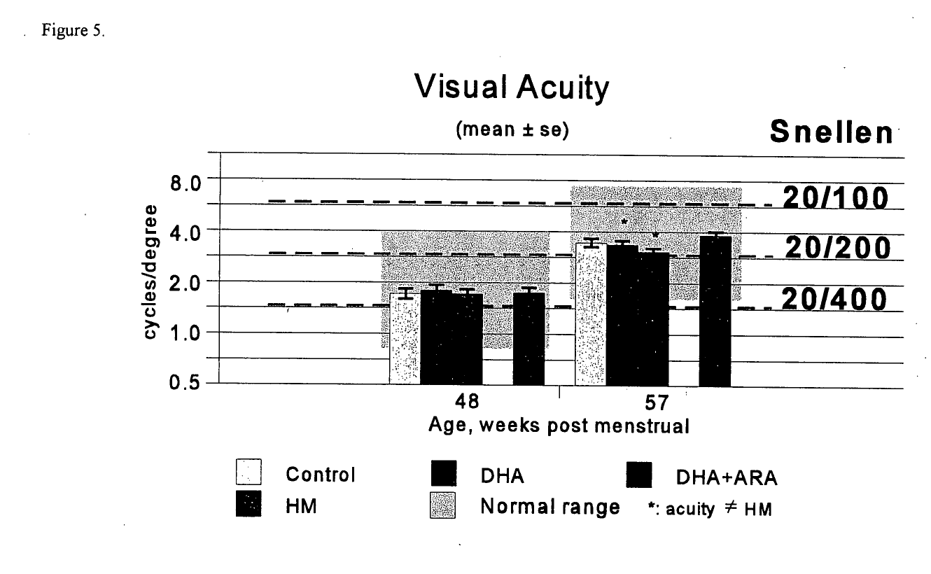

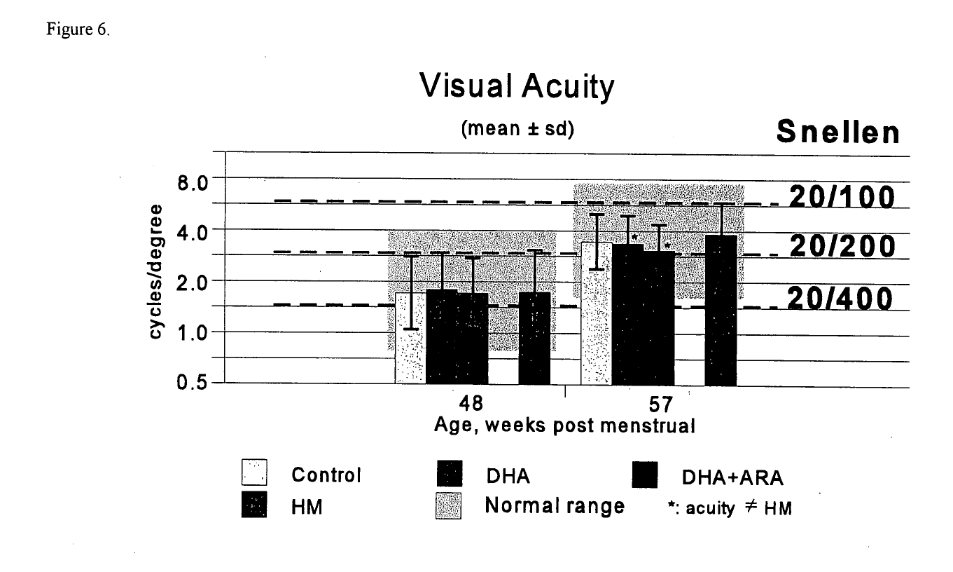

Visual acuity has reportedly been enhanced in studies where DHA supplemented formulas were fed to premature infants both in the hospital and continuing after discharge. In this study, visual acuity was measured about 3 months and then about 5 months after stopping study formula to determine whether a residual beneficial effect of at least half an octave might be observed. Although no difference in visual acuity was found among the formula groups at these times (See table 8 and FIG. 5), the acuity card method used, the length of study formula feeding, and/or the length of time not on study formula at the time of measurement may have precluded its detection. However, at 57 weeks post-conceptual age, the breast fed term infant group did have statistically higher visual acuity scores than the test formula groups. But even these differences were at most only 0.33 octave and were clinically insignificant (See FIG. 6). It is important to note that the breast fed infants continued to receive DHA and ARA during the 3-5 month follow-up period while the formula fed groups did not. Thus, this minor difference in performance was not unexpected based on previous study findings and on developmental differences between term and preterm infants even at the same gestational age.

Individual fatty acid levels were determined in the phosphatidylcholine and phosphatidylethanolamine fractions of red blood cells before formula feeding, at the conclusion of test formula feeding, and at 48 weeks post-conceptual age (See tables 9 and 10). The premature infant groups were comparable at the beginning of test formula feeding. At the conclusion of test formula feeding, individual fatty acid levels varied among the groups. DHA and ARA were statistically significantly higher in the respectively supplemented groups. Other fatty acid levels reflected the impact of the supplementation. No clinically significant alterations in fatty acid levels or metabolism were identified. After discontinuing study formula and consuming a diet without DHA or ARA for about 3 months, no differences in fatty acid levels among formula fed groups were detectable, except for phosphatidylethanolamine levels of 18:2 (range 8.9-9.3%) and DHA (range 3.2-4.1%) which differences were not identified as being clinically significant. However, the breast fed group shows statistically significant differences in 13 fatty acid levels compared to the formula fed infants. These differences are undoubtedly due to the differences in fatty acid composition of human milk and the term formulas including the lack of DHA and ARA in the latter.

Preterm infant complications were similar in all groups (See table 11). Over 80% of all infants were opthamologically examined and over 90% had ultrasound evaluation of their heads. Specifically, the incidence and severity of retinopathy of prematurity (ROP or retrolental fibroplasia/RLF) and the incidence of intraventricular hemorrhage or its complications did not differ among formula groups. No feeding group related complications were identified.

Serious adverse experiences did not differ (p=0.93) among the formula groups and were in the range of those expected in a premature infant population while on study formula: 6% in group C, 5% in group D, and 6% in group DA (See table 12). After the experimental formula phase, serious adverse experiences still did not differ among the preterm groups (See table 13): 13% in group C, 15% in group D, and 15% in group DA. However, the term infant breast fed group had significantly fewer serious adverse experiences (1%, p=0.002) as expected. Two infants reportedly suffered sudden infant death syndrome (SIDS), one in group C and one in group D; there was no significant difference in this complication among all four groups.

### CONCLUSIONS

We conclude that feeding 0.13% of calories as DHA from micro algal oil and feeding 0.13% of calories as DHA from micro algal oil plus 0.26% of calories as ARA from fungal oil in the matrix of premature infant formula to premature infants during the period of their initial hospitalization prior to 40 weeks post conceptual age is safe. These micro algal and fungal oil supplements do not result in any adverse effect on growth, clinical complications, or untoward events. Furthermore, this study reveals that growth benefits accrue to premature infants fed Enfamil Premature Formula supplemented with DHA and ARA from these sources compared to unsupplemented formula or formula supplemented with only DHA. No measurable benefit on visual acuity was identified when infants were tested at about 3 and 5 months after the supplemented formula was discontinued (2 and 4 months corrected age). However, providing human milk levels of intake of long chain polyunsaturated acids are warranted because they are critical to brain development and foster enhanced catch-up growth during this early development period.

TABLE 1
Birth Statistics of Premature Subjects

| | | | | p- |
|---|---|---|---|---|
| | n | Mean (std) | Range | value |
| Post-Conceptual Age (Weeks) | | | | |
| Control | 62 | 29.5 (1.7) | 25-33 | 0.076 |
| DHA | 66 | 30.0 (1.4) | 26-32 | |
| DHA + ARA | 66 | 29.7 (1.7) | 26-34 | |
| Birth Weight (g) | | | | |
| Control | 62 | 1233.1 (176.6) | 846-1560 | 0.25 |
| DHA | 66 | 1272.8 (168.1) | 900-1545 | |
| DHA + ARA | 66 | 1278.9 (177.6) | 910-1535 | |
| Birth Length (cm) | | | | |
| Control | 60 | 38.4 (2.3) | 34-43.75 | 0.62 |
| DHA | 66 | 38.6 (2.2) | 33-43.5 | |
| DHA + ARA | 66 | 38.7 (2.3) | 33-44 | |
| Birth Head Circumference | | | | |
| (cm) | | | | |
| Control | 61 | 26.9 (1.5) | 23.5-30.5 | 0.53 |
| DHA | 64 | 27.3 (2.1) | 22-37 | |
| DHA + ARA | 65 | 27.2 (1.6) | 23.5-30 | |

TABLE 2
Summary of Final Study Status

| | Regimen | | | | |
|---|---|---|---|---|---|
| | Control | DHA | DHA + ARA | HM | p-value |
| Immediate dropout, study formula | | 2 | 2 | | |
| never consumed | | | | | |
| Study Formula Phase* | | | | | |
| Completed | 52 (84%) | 59 (89%) | 62 (94%) | | 0.20 |
| Discontinued | 10 (16%) | 7 (11%) | 4 (6%) | | |
| Reason discontinued | | | | | |
| >96 cumulative hours NPO | 3 | 1 | | | |
| <28 days of intake >= 90 kcal/kg/day | 3 | 3 | | | |
| Complications unrelated to study | 1 | | | | |
| formula | | | | | |
| NEC or other GI disease | | 1 | 1 | | |
| Formula intolerance | | | 1 | | |
| Parents request | 2 | 2 | 1 | | |
| Not off oxygen prior to discharge | | | 1 | | |
| Protocol violation | 1 | | | | |
| Term Formula Phase** | | | | | |
| Completed | 45 (87%) | 47 (80%) | 53 (85%) | 77 (86%) | 0.74 |
| Discontinued | 7 (13%) | 12 (20%) | 9 (15%) | 13 (14%) | |

*The CRFs for 9709-003 (DHA) and 9743-304 (DHA) were marked discontinued because the subjects met the study formula intake criteria for only 27 days. These subjects are counted completed here because subjects at other sites with similar intakes were marked completed.
**Based on subjects who completed the Study Formula phase. During the Term Formula phase, subjects were fed marketed formula. Switching to a different marketed formula did not result in termination from the Term Formula phase.

TABLE 3
Weight Growth Rate During Study Formula Phase

| | | Least Square | Standard | | Comparison | Study | Gender | Gender-by-Regimen |
|---|---|---|---|---|---|---|---|---|
| Regimen | n | Mean | Error | Comparison | p-value* | p-value | p-value | p-value |
| Control | 60 | 30.7 | 1.1 | Control vs DHA | 0.967 | 0.00 | 0.17 | 0.87 |
| DHA | 65 | 33.3 | 1.1 | Control vs DHA + ARA | 0.998 | | | |
| DHA + ARA | 66 | 34.7 | 1.1 | | | | | |

*One-sided test of the null hypothesis: Test Mean >= Control Mean

TABLE 4
Weight at 40, 48, and 57 Weeks Post-Conceptual Age

| Weeks | | | Least | | | | | | Gender-by- |
|---|---|---|---|---|---|---|---|---|---|
| Post-Conceptual | | | Square | Standard | | Comparison | Study | Gender | Regimen |
| Age | Regimen | n | Mean | Error | Comparison | p-value* | p-value | p-value | p-value |
| 40 | Control | 52 | 3075.3 | 67.9 | Control vs DHA | 0.388 | 0.59 | 0.45 | 1.00 |
| | DHA | 54 | 3051.4 | 66.8 | Control vs DHA + ARA | 0.931 | | | |
| | DHA + ARA | 59 | 3198.2 | 62.9 | HM vs DHA | 0.000 | | | |
| | HM | 90 | 3437.7 | 60.6 | HM vs DHA + ARA | 0.001 | | | |
| | | | | | HM vs Control | 0.000 | | | |
| 48 | Control | 53 | 4711.0 | 94.6 | Control vs DHA | 0.360 | 0.58 | 0.13 | 0.29 |
| | DHA | 51 | 4663.8 | 97.3 | Control vs DHA + ARA | 0.995 | | | |
| | DHA + ARA | 57 | 5039.1 | 93.0 | HM vs DHA | 0.000 | | | |
| | HM | 81 | 5181.5 | 85.9 | HM vs DHA + ARA | 0.114 | | | |
| | | | | | HM vs Control | 0.000 | | | |
| 57 | Control | 47 | 6045.4 | 139.5 | Control vs DHA | 0.371 | 0.58 | 0.29 | 0.33 |
| | DHA | 49 | 5987.2 | 137.6 | Control vs DHA + ARA | 0.940 | | | |
| | DHA + ARA | 55 | 6312.9 | 127.9 | HM vs DHA | 0.005 | | | |
| | HM | 76 | 6405.0 | 126.7 | HM vs DMA + ARA | 0.278 | | | |
| | | | | | HM vs Control | 0.014 | | | |

*One-sided test of the null hypothesis: Test Mean >= Control Mean

TABLE 5
Post-hoc Analysis of Weight

| | | Two-sided |
|---|---|---|
| Time | Comparison | p-value |
| Weight Gain During Study | C vs. DHA | 0.067 |
| Formula Phase | C vs. DHA + ARA | 0.004 |
| | DHA vs. DHA + ARA | 0.30 |
| Weight at 40 Weeks pca | C vs. DHA | 0.78 |
| | C vs. DHA + ARA | 0.14 |
| | DHA vs. DHA + ARA | 0.074 |
| | HM vs. DHA | <0.001 |
| | HM vs. DHA + ARA | 0.002 |
| | HM vs. C | <0.001 |
| Weight at 48 Weeks pca | C vs. DHA | 0.72 |
| | C vs. DHA + ARA | 0.011 |
| | DHA vs. DHA + ARA | 0.004 |
| | HM vs. DHA | <0.001 |
| | HM vs. DHA + ARA | 0.23 |
| | HM vs. C | <0.001 |
| Weight at 57 Weeks pca | C vs. DHA | 0.74 |
| | C vs. DHA + ARA | 0.12 |
| | DHA vs. DHA + ARA | 0.057 |
| | HM vs. DHA | 0.010 |
| | HM vs. DHA + ARA | 0.56 |
| | HM vs. C | 0.028 |

TABLE 6
Length at 40, 48, and 57 Weeks Post-Conceptual Age

| Weeks | | | | | | | | | | Gender- |
|---|---|---|---|---|---|---|---|---|---|---|
| Post- | | | Least | | | | | | | by- |
| Conceptual | | | Square | Standard | Regimen | Pairwise | Pairwise | Study | Gender | Regimen |
| Age | Regimen | n | Mean | Error | p-value | Comparison | p-value | p-value | p-value | p-value |
| 40 | Control | 52 | 48.4 | 0.4 | 0.000 | Control vs DHA | 0.242 | 0.03 | 0.88 | 0.63 |
| | DHA | 54 | 47.8 | 0.4 | | Control vs DHA + ARA | 0.233 | | | |
| | DHA + ARA | 58 | 49.0 | 0.4 | | HM vs DHA | 0.000 | | | |
| | HM | 89 | 50.6 | 0.4 | | HM vs DHA + ARA | 0.000 | | | |
| | | | | | | Control vs HM | 0.000 | | | |
| | | | | | | DHA vs DHA + ARA | 0.017 | | | |
| 48 | Control | 53 | 54.7 | 0.3 | 0.000 | Control vs DHA | 0.824 | 0.00 | 0.14 | 0.52 |
| | DHA | 52 | 54.6 | 0.3 | | Control vs DHA + ARA | 0.079 | | | |
| | DHA + ARA | 57 | 55.5 | 0.3 | | HM vs DHA | 0.000 | | | |
| | HM | 81 | 57.4 | 0.3 | | HM vs DHA + ARA | 0.000 | | | |
| | | | | | | Control vs HM | 0.000 | | | |
| | | | | | | DHA vs DHA + ARA | 0.050 | | | |
| 57 | Control | 47 | 60.7 | 0.4 | 0.000 | Control vs DHA | 0.615 | 0.00 | 0.02 | 0.84 |
| | DHA | 49 | 60.5 | 0.4 | | Control vs DHA + ARA | 0.236 | | | |
| | DHA + ARA | 54 | 61.3 | 0.3 | | HM vs DHA | 0.000 | | | |
| | HM | 76 | 62.4 | 0.3 | | HM vs DHA + ARA | 0.006 | | | |
| | | | | | | Control vs HM | 0.000 | | | |
| | | | | | | DHA vs DHA + ARA | 0.087 | | | |

TABLE 7
Head Circumference at 40, 48, and 57 Weeks Post-Conceptual Age

| Weeks | | | | | | | | | | Gender- |
|---|---|---|---|---|---|---|---|---|---|---|
| Post- | | | Least | | | | | | | by- |
| Conceptual | | | Square | Standard | Regimen | Pairwise | Pairwise | Study | Gender | Regimen |
| Age | Regimen | n | Mean | Error | p-value | Comparison | p-value | p-value | p-value | p-value |
| 40 | Control | 51 | 35.4 | 0.2 | 0.000 | Control vs DHA | 0.931 | 0.91 | 0.00 | 0.38 |
| | DHA | 53 | 35.4 | 0.2 | | Control vs DHA + ARA | 0.900 | | | |
| | DHA + ARA | 58 | 35.5 | 0.2 | | HM vs DHA | 0.000 | | | |
| | HM | 85 | 34.5 | 0.2 | | HM vs DHA + ARA | 0.000 | | | |
| | | | | | | Control vs HM | 0.000 | | | |
| | | | | | | DHA vs DHA + ARA | 0.829 | | | |
| 48 | Control | 52 | 39.1 | 0.2 | 0.983 | | | 0.81 | 0.00 | 1.00 |
| | DHA | 51 | 39.0 | 0.2 | | | | | | |
| | DHA + ARA | 56 | 39.0 | 0.2 | | | | | | |
| | HM | 81 | 39.0 | 0.1 | | | | | | |
| 57 | Control | 47 | 41.9 | 0.2 | 0.689 | | | 0.64 | 0.00 | 0.85 |
| | DHA | 49 | 41.6 | 0.2 | | | | | | |
| | DHA + ARA | 53 | 41.7 | 0.2 | | | | | | |
| | HM | 76 | 41.7 | 0.2 | | | | | | |

TABLE 8
Visual Acuity at 48 and 57 Weeks Post-Conceptual Age

| | | | | Least Square | | | | | |
|---|---|---|---|---|---|---|---|---|---|
| Weeks | | | | Mean (log base 2 | Standard | | | | |
| Post-Conceptual | | | Geometric mean | cycles/ | Error | Regimen | Pairwise | Pairwise | Study |
| Age | Regimen | n | (cycles/deg) | deg) | (octaves) | p-value | Comparison | p-value | p-value |
| 48 | Control | 51 | 1.72 | 0.78 | 0.10 | 0.950 | | | 0.000 |
| | DHA | 50 | 1.80 | 0.85 | 0.10 | | | | |
| | DHA + ARA | 57 | 1.72 | 0.78 | 0.09 | | | | |
| | HM | 81 | 1.75 | 0.81 | 0.09 | | | | |
| 57 | Control | 46 | 3.47 | 1.79 | 0.08 | 0.004 | Control vs DHA | 0.697 | 0.000 |
| | DHA | 47 | 3.37 | 1.75 | 0.08 | | Control vs DHA + ARA | 0.071 | |
| | DHA + ARA | 55 | 3.06 | 1.61 | 0.07 | | HM vs DHA | 0.042 | |
| | HM | 77 | 3.85 | 1.94 | 0.07 | | HM vs DHA + ARA | 0.000 | |
| | | | | | | | Control vs HM | 0.113 | |
| | | | | | | | DHA vs DHA + ARA | 0.158 | |

TABLE 9
Red Blood Cell Phosphatidylcholine Fatty Acids

| | | | | Arithmetic | Standard | | Regimen | Pairwise | |
|---|---|---|---|---|---|---|---|---|---|
| Time | Fatty Acid | Regimen | n | Mean | Error | Median | p-value | Comparison | Pairwise p-value |
| Study Form Initiation | 12:0 | Control | 52 | 0.081 | 0.019 | 0.036 | 0.762 | | |
| | | DHA | 58 | 0.066 | 0.013 | 0.030 | | | |
| | | DHA + ARA | 61 | 0.057 | 0.009 | 0.031 | | | |
| Study Form Initiation | 14:0 | Control | 52 | 0.623 | 0.036 | 0.599 | 0.559 | | |
| | | DHA | 58 | 0.663 | 0.031 | 0.686 | | | |
| | | DHA + ARA | 61 | 0.661 | 0.031 | 0.656 | | | |
| Study Form Initiation | 14:1 | Control | 52 | 0.045 | 0.009 | 0.021 | 0.165 | | |
| | | DHA | 58 | 0.026 | 0.005 | 0.016 | | | |
| | | DHA + ARA | 61 | 0.035 | 0.006 | 0.018 | | | |
| Study Form Initiation | 16:0 | Control | 52 | 36.706 | 0.540 | 36.594 | 0.884 | | |
| | | DHA | 58 | 36.363 | 0.462 | 35.578 | | | |
| | | DHA + ARA | 61 | 36.877 | 0.445 | 35.987 | | | |
| Study Form Initiation | 16:1 | Control | 52 | 0.940 | 0.049 | 0.845 | 0.441 | | |
| | | DHA | 58 | 0.981 | 0.050 | 0.976 | | | |
| | | DHA + ARA | 61 | 1.094 | 0.064 | 0.931 | | | |
| Study Form Initiation | 18:0 | Control | 52 | 11.660 | 0.243 | 11.468 | 0.243 | | |
| | | DHA | 58 | 11.402 | 0.238 | 11.201 | | | |
| | | DHA + ARA | 61 | 11.016 | 0.192 | 11.174 | | | |
| Study Form Initiation | 18:1 | Control | 52 | 17.053 | 0.298 | 17.308 | 0.679 | | |
| | | DHA | 58 | 17.219 | 0.391 | 16.935 | | | |
| | | DHA + ARA | 61 | 17.256 | 0.271 | 16.988 | | | |
| Study Form Initiation | 18:2 | Control | 52 | 18.614 | 0.525 | 18.952 | 0.830 | | |
| | | DHA | 58 | 18.631 | 0.505 | 19.603 | | | |
| | | DHA + ARA | 61 | 18.573 | 0.466 | 18.824 | | | |
| Study Form Initiation | 18:3n6 | Control | 52 | 0.120 | 0.008 | 0.116 | 0.034 | Control vs DHA | 0.196 |
| | | DHA | 58 | 0.136 | 0.008 | 0.130 | | Control vs DHA + ARA | 0.010 |
| | | DHA + ARA | 61 | 0.150 | 0.009 | 0.134 | | DHA vs DHA + ARA | 0.176 |
| Study Form Initiation | 20:0 | Control | 52 | 0.399 | 0.050 | 0.224 | 0.647 | | |
| | | DHA | 58 | 0.337 | 0.035 | 0.236 | | | |
| | | DHA + ARA | 61 | 0.310 | 0.037 | 0.188 | | | |
| Study Form Initiation | 18:3n3 | Control | 52 | 0.315 | 0.033 | 0.246 | 0.234 | | |
| | | DHA | 58 | 0.257 | 0.014 | 0.246 | | | |
| | | DHA + ARA | 61 | 0.233 | 0.010 | 0.216 | | | |
| Study Form Initiation | 20:1 | Control | 52 | 0.287 | 0.020 | 0.262 | 0.723 | | |
| | | DHA | 58 | 0.287 | 0.015 | 0.281 | | | |
| | | DHA + ARA | 61 | 0.268 | 0.011 | 0.269 | | | |
| Study Form Initiation | 18:4 | Control | 52 | 0.017 | 0.003 | 0.000 | 0.290 | | |
| | | DHA | 58 | 0.025 | 0.004 | 0.017 | | | |
| | | DHA + ARA | 61 | 0.017 | 0.003 | 0.008 | | | |
| Study Form Initiation | 20:2n6 | Control | 52 | 0.632 | 0.025 | 0.632 | 0.673 | | |
| | | DHA | 58 | 0.628 | 0.025 | 0.640 | | | |
| | | DHA + ARA | 61 | 0.602 | 0.021 | 0.614 | | | |
| Study Form Initiation | 20:3n6 | Control | 52 | 2.144 | 0.098 | 2.096 | 0.507 | | |
| | | DHA | 58 | 2.208 | 0.080 | 2.296 | | | |
| | | DHA + ARA | 61 | 2.218 | 0.074 | 2.135 | | | |
| Study Form Initiation | 20:4n6 | Control | 52 | 7.657 | 0.262 | 8.124 | 0.819 | | |
| | | DHA | 58 | 8.164 | 0.347 | 7.876 | | | |
| | | DHA + ARA | 61 | 8.090 | 0.310 | 8.207 | | | |
| Study Form Initiation | 22:1 | Control | 52 | 0.106 | 0.010 | 0.105 | 0.155 | | |
| | | DHA | 58 | 0.127 | 0.010 | 0.130 | | | |
| | | DHA + ARA | 61 | 0.126 | 0.010 | 0.139 | | | |
| Study Form Initiation | 20:5n3 | Control | 52 | 0.351 | 0.057 | 0.298 | 0.911 | | |
| | | DHA | 58 | 0.322 | 0.015 | 0.302 | | | |
| | | DHA + ARA | 61 | 0.321 | 0.015 | 0.329 | | | |
| Study Form Initiation | 22:4n6 | Control | 52 | 0.578 | 0.144 | 0.423 | 0.331 | | |
| | | DHA | 58 | 0.493 | 0.030 | 0.481 | | | |
| | | DHA + ARA | 61 | 0.443 | 0.021 | 0.425 | | | |
| Study Form Initiation | 24:1 | Control | 52 | 0.208 | 0.054 | 0.075 | 0.665 | | |
| | | DHA | 58 | 0.115 | 0.019 | 0.084 | | | |
| | | DHA + ARA | 61 | 0.180 | 0.056 | 0.096 | | | |
| Study Form Initiation | 22:5n6 | Control | 52 | 0.266 | 0.020 | 0.232 | 0.923 | | |
| | | DHA | 58 | 0.259 | 0.017 | 0.239 | | | |
| | | DHA + ARA | 61 | 0.265 | 0.018 | 0.256 | | | |
| Study Form Initiation | 22:4n3 | Control | 52 | 0.000 | 0.000 | 0.000 | 0.199 | | |
| | | DHA | 58 | 0.001 | 0.001 | 0.000 | | | |
| | | DHA + ARA | 61 | 0.002 | 0.001 | 0.000 | | | |
| Study Form Initiation | 22:5n3 | Control | 52 | 0.213 | 0.019 | 0.203 | 0.885 | | |
| | | DHA | 58 | 0.215 | 0.013 | 0.195 | | | |
| | | DHA + ARA | 61 | 0.203 | 0.010 | 0.193 | | | |
| Study Form Initiation | 22:6n3 | Control | 52 | 0.984 | 0.051 | 1.000 | 0.858 | | |
| | | DHA | 58 | 1.075 | 0.053 | 1.034 | | | |
| | | DHA + ARA | 61 | 1.006 | 0.050 | 0.970 | | | |
| Study Form Termination | 12:0 | Control | 53 | 0.100 | 0.026 | 0.035 | 0.843 | | |
| | | DHA | 56 | 0.111 | 0.042 | 0.031 | | | |
| | | DHA + ARA | 59 | 0.064 | 0.012 | 0.032 | | | |
| Study Form Termination | 14:0 | Control | 53 | 0.808 | 0.039 | 0.806 | 0.834 | | |
| | | DHA | 56 | 0.781 | 0.035 | 0.783 | | | |
| | | DHA + ARA | 59 | 0.755 | 0.036 | 0.758 | | | |
| Study Form Termination | 14:1 | Control | 53 | 0.047 | 0.008 | 0.033 | 0.155 | | |
| | | DHA | 56 | 0.036 | 0.009 | 0.015 | | | |
| | | DHA + ARA | 59 | 0.036 | 0.007 | 0.018 | | | |
| Study Form Termination | 16:0 | Control | 53 | 35.837 | 0.512 | 34.798 | 0.767 | | |
| | | DHA | 56 | 35.560 | 0.595 | 34.841 | | | |
| | | DHA + ARA | 59 | 35.069 | 0.584 | 33.890 | | | |
| Study Form Termination | 16:1 | Control | 53 | 0.566 | 0.026 | 0.526 | 0.013 | Control vs DHA | 0.118 |
| | | DHA | 56 | 0.594 | 0.042 | 0.475 | | Control vs DHA + ARA | 0.003 |
| | | DHA + ARA | 59 | 0.526 | 0.029 | 0.472 | | DHA vs DHA + ARA | 0.152 |
| Study Form Termination | 18:0 | Control | 53 | 13.972 | 0.261 | 14.197 | 0.886 | | |
| | | DHA | 56 | 14.065 | 0.237 | 13.867 | | | |
| | | DHA + ARA | 59 | 14.341 | 0.253 | 14.108 | | | |
| Study Form Termination | 18:1 | Control | 53 | 14.456 | 0.277 | 14.291 | 0.686 | | |
| | | DHA | 56 | 14.116 | 0.272 | 13.998 | | | |
| | | DHA + ARA | 59 | 14.344 | 0.380 | 14.218 | | | |
| Study Form Termination | 18:2 | Control | 53 | 21.673 | 0.340 | 21.506 | 0.001 | Control vs DHA | 0.600 |
| | | DHA | 56 | 22.045 | 0.457 | 22.517 | | Control vs DHA + ARA | 0.005 |
| | | DHA + ARA | 59 | 19.899 | 0.337 | 20.662 | | DHA vs DHA + ARA | 0.001 |
| Study Form Termination | 18:3n6 | Control | 53 | 0.080 | 0.006 | 0.074 | 0.527 | | |
| | | DHA | 56 | 0.088 | 0.009 | 0.076 | | | |
| | | DHA + ARA | 59 | 0.087 | 0.013 | 0.066 | | | |
| Study Form Termination | 20:0 | Control | 53 | 0.504 | 0.050 | 0.392 | 0.424 | | |
| | | DHA | 56 | 0.472 | 0.053 | 0.281 | | | |
| | | DHA + ARA | 59 | 0.430 | 0.049 | 0.251 | | | |
| Study Form Termination | 18:3n3 | Control | 53 | 0.321 | 0.020 | 0.283 | 0.031 | Control vs DHA | 0.503 |
| | | DHA | 56 | 0.335 | 0.030 | 0.285 | | Control vs DHA + ARA | 0.068 |
| | | DHA + ARA | 59 | 0.273 | 0.009 | 0.256 | | DHA vs DHA + ARA | 0.011 |
| Study Form Termination | 20:1 | Control | 53 | 0.318 | 0.014 | 0.302 | 0.149 | | |
| | | DHA | 56 | 0.300 | 0.013 | 0.283 | | | |
| | | DHA + ARA | 59 | 0.307 | 0.013 | 0.283 | | | |
| Study Form Termination | 18:4 | Control | 53 | 0.022 | 0.004 | 0.015 | 0.672 | | |
| | | DHA | 56 | 0.022 | 0.003 | 0.018 | | | |
| | | DHA + ARA | 59 | 0.014 | 0.002 | 0.008 | | | |
| Study Form Termination | 20:2n6 | Control | 53 | 0.893 | 0.026 | 0.910 | 0.051 | | |
| | | DHA | 56 | 0.880 | 0.023 | 0.873 | | | |
| | | DHA + ARA | 59 | 0.824 | 0.022 | 0.821 | | | |
| Study Form Termination | 20:3n6 | Control | 53 | 2.032 | 0.073 | 2.091 | 0.208 | | |
| | | DHA | 56 | 2.017 | 0.070 | 2.043 | | | |
| | | DHA + ARA | 59 | 1.908 | 0.064 | 1.904 | | | |
| Study Form Termination | 20:4n6 | Control | 53 | 6.046 | 0.240 | 6.029 | 0.000 | Control vs DHA | 0.097 |
| | | DHA | 56 | 5.774 | 0.220 | 5.892 | | Control vs DHA + ARA | 0.000 |
| | | DHA + ARA | 59 | 8.465 | 0.255 | 8.891 | | DHA vs DHA + ARA | 0.000 |
| Study Form Termination | 22:1 | Control | 53 | 0.117 | 0.010 | 0.125 | 0.946 | | |
| | | DHA | 56 | 0.110 | 0.009 | 0.114 | | | |
| | | DHA + ARA | 59 | 0.115 | 0.011 | 0.104 | | | |
| Study Form Termination | 20:5n3 | Control | 53 | 0.214 | 0.022 | 0.189 | 0.000 | Control vs DHA | 0.004 |
| | | DHA | 56 | 0.246 | 0.012 | 0.233 | | Control vs DHA + ARA | 0.108 |
| | | DHA + ARA | 59 | 0.186 | 0.014 | 0.169 | | DHA vs DHA + ARA | 0.000 |
| Study Form Termination | 22:4n6 | Control | 53 | 0.484 | 0.048 | 0.390 | 0.093 | | |
| | | DHA | 56 | 0.489 | 0.061 | 0.426 | | | |
| | | DHA + ARA | 59 | 0.496 | 0.027 | 0.487 | | | |
| Study Form Termination | 24:1 | Control | 53 | 0.127 | 0.039 | 0.062 | 0.303 | | |
| | | DHA | 56 | 0.143 | 0.036 | 0.086 | | | |
| | | DHA + ARA | 59 | 0.177 | 0.040 | 0.089 | | | |
| Study Form Termination | 22:5n6 | Control | 53 | 0.181 | 0.013 | 0.163 | 0.006 | Control vs DHA | 0.005 |
| | | DHA | 56 | 0.145 | 0.011 | 0.133 | | Control vs DHA + ARA | 0.895 |
| | | DHA + ARA | 59 | 0.172 | 0.009 | 0.165 | | DHA vs DHA + ARA | 0.006 |
| Study Form Termination | 22:4n3 | Control | 53 | 0.001 | 0.001 | 0.000 | 0.359 | | |
| | | DHA | 56 | 0.001 | 0.001 | 0.000 | | | |
| | | DHA + ARA | 59 | 0.003 | 0.002 | 0.000 | | | |
| Study Form Termination | 22:5n3 | Control | 53 | 0.306 | 0.019 | 0.289 | 0.221 | | |
| | | DHA | 56 | 0.293 | 0.026 | 0.260 | | | |
| | | DHA + ARA | 59 | 0.265 | 0.013 | 0.255 | | | |
| Study Form Termination | 22:6n3 | Control | 53 | 0.895 | 0.072 | 0.812 | 0.000 | Control vs DHA | 0.000 |
| | | DHA | 56 | 1.380 | 0.063 | 1.352 | | Control vs DHA + ARA | 0.000 |
| | | DHA + ARA | 59 | 1.244 | 0.049 | 1.259 | | DHA vs DHA + ARA | 0.141 |
| 48 Weeks PCA | 12:0 | Control | 37 | 0.032 | 0.005 | 0.026 | 0.729 | | |
| | | DHA | 32 | 0.028 | 0.006 | 0.016 | | | |
| | | DHA + ARA | 38 | 0.026 | 0.004 | 0.021 | | | |
| | | HM | 56 | 0.059 | 0.016 | 0.020 | | | |
| 48 Weeks PCA | 14:0 | Control | 37 | 0.402 | 0.039 | 0.331 | 0.943 | | |
| | | DHA | 32 | 0.353 | 0.032 | 0.324 | | | |
| | | DHA + ARA | 38 | 0.353 | 0.024 | 0.328 | | | |
| | | HM | 56 | 0.381 | 0.026 | 0.335 | | | |
| 48 Weeks PCA | 14:1 | Control | 37 | 0.025 | 0.006 | 0.013 | 0.448 | | |
| | | DHA | 32 | 0.026 | 0.007 | 0.011 | | | |
| | | DHA + ARA | 38 | 0.026 | 0.006 | 0.015 | | | |
| | | HM | 56 | 0.024 | 0.003 | 0.020 | | | |
| 48 Weeks PCA | 16:0 | Control | 37 | 34.627 | 0.577 | 34.319 | 0.000 | Control vs DHA | 0.527 |
| | | DHA | 32 | 35.272 | 0.689 | 34.473 | | Control vs DHA + ARA | 0.593 |
| | | DHA + ARA | 38 | 34.802 | 0.506 | 34.165 | | HM vs DHA | 0.000 |
| | | HM | 56 | 33.037 | 0.506 | 32.228 | | HM vs DHA + ARA | 0.000 |
| | | | | | | | | Control vs HM | 0.000 |
| | | | | | | | | DHA vs DHA + ARA | 0.906 |
| 48 Weeks PCA | 16:1 | Control | 37 | 0.435 | 0.043 | 0.338 | 0.000 | Control vs DHA | 0.524 |
| | | DHA | 32 | 0.380 | 0.023 | 0.352 | | Control vs DHA + ARA | 0.467 |
| | | DHA + ARA | 38 | 0.395 | 0.024 | 0.368 | | HM vs DHA | 0.000 |
| | | HM | 56 | 0.507 | 0.020 | 0.473 | | HM vs DHA + ARA | 0.006 |
| | | | | | | | | Control vs HM | 0.000 |
| | | | | | | | | DHA vs DHA + ARA | 0.183 |
| 48 Weeks PCA | 18:0 | Control | 37 | 13.016 | 0.313 | 12.759 | 0.000 | Control vs DHA | 0.760 |
| | | DHA | 32 | 12.944 | 0.249 | 12.786 | | Control vs DHA + ARA | 0.889 |
| | | DHA + ARA | 38 | 12.804 | 0.235 | 12.793 | | HM vs DHA | 0.000 |
| | | HM | 56 | 14.583 | 0.287 | 14.729 | | HM vs DHA + ARA | 0.000 |
| | | | | | | | | Control vs HM | 0.000 |
| | | | | | | | | DHA vs DHA + ARA | 0.661 |
| 48 Weeks PCA | 18:1 | Control | 37 | 17.894 | 0.453 | 18.636 | 0.256 | | |
| | | DHA | 32 | 17.766 | 0.429 | 18.492 | | | |
| | | DHA + ARA | 38 | 17.850 | 0.289 | 18.227 | | | |
| | | HM | 56 | 18.662 | 0.305 | 18.727 | | | |
| 48 Weeks PCA | 18:2 | Control | 37 | 23.469 | 0.518 | 23.552 | 0.000 | Control vs DHA | 0.840 |
| | | DHA | 32 | 23.538 | 0.516 | 23.717 | | Control vs DHA + ARA | 0.527 |
| | | DHA + ARA | 38 | 23.738 | 0.422 | 23.839 | | HM vs DHA | 0.000 |
| | | HM | 56 | 18.650 | 0.344 | 18.482 | | HM vs DHA + ARA | 0.000 |
| | | | | | | | | Control vs HM | 0.000 |
| | | | | | | | | DHA vs DHA + ARA | 0.685 |
| 48 Weeks PCA | 18:3n6 | Control | 37 | 0.071 | 0.008 | 0.061 | 0.002 | Control vs DHA | 0.950 |
| | | DHA | 32 | 0.069 | 0.005 | 0.067 | | Control vs DHA + ARA | 0.774 |
| | | DHA + ARA | 38 | 0.069 | 0.006 | 0.062 | | HM vs DHA | 0.004 |
| | | HM | 56 | 0.042 | 0.004 | 0.039 | | HM vs DHA + ARA | 0.001 |
| | | | | | | | | Control vs HM | 0.003 |
| | | | | | | | | DHA vs DHA + ARA | 0.831 |
| 48 Weeks PCA | 20:0 | Control | 37 | 0.348 | 0.075 | 0.197 | 0.785 | | |
| | | DHA | 32 | 0.339 | 0.061 | 0.206 | | | |
| | | DHA + ARA | 38 | 0.304 | 0.061 | 0.172 | | | |
| | | HM | 56 | 0.409 | 0.044 | 0.215 | | | |
| 48 Weeks PCA | 18:3n3 | Control | 37 | 0.222 | 0.019 | 0.182 | 0.001 | Control vs DHA | 0.812 |
| | | DHA | 32 | 0.211 | 0.015 | 0.182 | | Control vs DHA + ARA | 0.918 |
| | | DHA + ARA | 38 | 0.203 | 0.010 | 0.190 | | HM vs DHA | 0.001 |
| | | HM | 56 | 0.182 | 0.022 | 0.120 | | HM vs DHA + ARA | 0.002 |
| | | | | | | | | Control vs HM | 0.001 |
| | | | | | | | | DHA vs DHA + ARA | 0.737 |
| 48 Weeks PCA | 20:1 | Control | 37 | 0.418 | 0.019 | 0.420 | 0.000 | Control vs DHA | 0.579 |
| | | DHA | 32 | 0.406 | 0.025 | 0.435 | | Control vs DHA + ARA | 0.588 |
| | | DHA + ARA | 38 | 0.382 | 0.016 | 0.375 | | HM vs DHA | 0.001 |
| | | HM | 56 | 0.311 | 0.014 | 0.309 | | HM vs DHA + ARA | 0.001 |
| | | | | | | | | Control vs HM | 0.000 |
| | | | | | | | | DHA vs DHA + ARA | 0.974 |
| 48 Weeks PCA | 18:4 | Control | 37 | 0.018 | 0.005 | 0.000 | 0.010 | Control vs DHA | 0.822 |
| | | DHA | 32 | 0.016 | 0.004 | 0.000 | | Control vs DHA + ARA | 0.161 |
| | | DHA + ARA | 38 | 0.007 | 0.002 | 0.000 | | HM vs DHA | 0.039 |
| | | HM | 56 | 0.024 | 0.004 | 0.015 | | HM vs DHA + ARA | 0.001 |
| | | | | | | | | Control vs HM | 0.054 |
| | | | | | | | | DHA vs DHA + ARA | 0.262 |
| 48 Weeks PCA | 20:2n6 | Control | 37 | 0.543 | 0.023 | 0.537 | 0.629 | | |
| | | DHA | 32 | 0.557 | 0.032 | 0.543 | | | |
| | | DHA + ARA | 38 | 0.636 | 0.053 | 0.550 | | | |
| | | HM | 56 | 0.560 | 0.014 | 0.531 | | | |
| 48 Weeks PCA | 20:3n6 | Control | 37 | 1.709 | 0.086 | 1.741 | 0.000 | Control vs DHA | 0.610 |
| | | DHA | 32 | 1.702 | 0.073 | 1.684 | | Control vs DHA + ARA | 0.735 |
| | | DHA + ARA | 38 | 1.844 | 0.090 | 1.717 | | HM vs DHA | 0.000 |
| | | HM | 56 | 2.265 | 0.086 | 2.166 | | HM vs DHA + ARA | 0.000 |
| | | | | | | | | Control vs HM | 0.000 |
| | | | | | | | | DHA vs DHA + ARA | 0.405 |
| 48 Weeks PCA | 20:4n6 | Control | 37 | 4.738 | 0.255 | 4.736 | 0.000 | Control vs DHA | 0.508 |
| | | DHA | 32 | 4.475 | 0.196 | 4.499 | | Control vs DHA + ARA | 0.805 |
| | | DHA + ARA | 38 | 4.550 | 0.185 | 4.746 | | HM vs DHA | 0.000 |
| | | HM | 56 | 7.408 | 0.250 | 7.666 | | HM vs DHA + ARA | 0.000 |
| | | | | | | | | Control vs HM | 0.000 |
| | | | | | | | | DHA vs DHA + ARA | 0.672 |
| 48 Weeks PCA | 22:1 | Control | 37 | 0.166 | 0.036 | 0.131 | 0.664 | | |
| | | DHA | 32 | 0.116 | 0.014 | 0.118 | | | |
| | | DHA + ARA | 38 | 0.131 | 0.024 | 0.105 | | | |
| | | HM | 56 | 0.160 | 0.030 | 0.104 | | | |
| 48 Weeks PCA | 20:5n3 | Control | 37 | 0.102 | 0.015 | 0.077 | 0.000 | Control vs DHA | 0.633 |
| | | DHA | 32 | 0.084 | 0.006 | 0.083 | | Control vs DHA + ARA | 0.086 |
| | | DHA + ARA | 38 | 0.099 | 0.009 | 0.078 | | HM vs DHA | 0.000 |
| | | HM | 56 | 0.138 | 0.009 | 0.123 | | HM vs DHA + ARA | 0.000 |
| | | | | | | | | Control vs HM | 0.000 |
| | | | | | | | | DHA vs DHA + ARA | 0.239 |
| 48 Weeks PCA | 22:4n6 | Control | 37 | 0.426 | 0.059 | 0.373 | 0.244 | | |
| | | DHA | 32 | 0.382 | 0.029 | 0.417 | | | |
| | | DHA + ARA | 38 | 0.440 | 0.054 | 0.384 | | | |
| | | HM | 56 | 0.406 | 0.022 | 0.377 | | | |
| 48 Weeks PCA | 24:1 | Control | 37 | 0.247 | 0.070 | 0.112 | 0.000 | Control vs DHA | 0.337 |
| | | DHA | 32 | 0.210 | 0.062 | 0.116 | | Control vs DHA + ARA | 0.247 |
| | | DHA + ARA | 38 | 0.179 | 0.055 | 0.108 | | HM vs DHA | 0.000 |
| | | HM | 56 | 0.115 | 0.020 | 0.079 | | HM vs DHA + ARA | 0.000 |
| | | | | | | | | Control vs HM | 0.000 |
| | | | | | | | | DHA vs DHA + ARA | 0.878 |
| 48 Weeks PCA | 22:5n6 | Control | 37 | 0.210 | 0.016 | 0.212 | 0.000 | Control vs DHA | 0.505 |
| | | DHA | 32 | 0.189 | 0.012 | 0.186 | | Control vs DHA + ARA | 0.647 |
| | | DHA + ARA | 38 | 0.231 | 0.022 | 0.198 | | HM vs DHA | 0.000 |
| | | HM | 56 | 0.264 | 0.016 | 0.265 | | HM vs DHA + ARA | 0.001 |
| | | | | | | | | Control vs HM | 0.000 |
| | | | | | | | | DHA vs DHA + ARA | 0.270 |
| 48 Weeks PCA | 22:4n3 | Control | 37 | 0.000 | 0.000 | 0.000 | 1.000 | | |
| | | DHA | 32 | 0.000 | 0.000 | 0.000 | | | |
| | | DHA + ARA | 38 | 0.000 | 0.000 | 0.000 | | | |
| | | HM | 56 | 0.000 | 0.000 | 0.000 | | | |
| 48 Weeks PCA | 22:5n3 | Control | 37 | 0.286 | 0.029 | 0.260 | 0.000 | Control vs DHA | 0.598 |
| | | DHA | 32 | 0.253 | 0.017 | 0.251 | | Control vs DHA + ARA | 0.759 |
| | | DHA + ARA | 38 | 0.268 | 0.026 | 0.256 | | HM vs DHA | 0.000 |
| | | HM | 56 | 0.339 | 0.018 | 0.314 | | HM vs DHA + ARA | 0.000 |
| | | | | | | | | Control vs HM | 0.000 |
| | | | | | | | | DHA vs DHA + ARA | 0.817 |
| 48 Weeks PCA | 22:6n3 | Control | 37 | 0.595 | 0.047 | 0.569 | 0.000 | Control vs DHA | 0.111 |
| | | DHA | 32 | 0.685 | 0.048 | 0.676 | | Control vs DHA + ARA | 0.052 |
| | | DHA + ARA | 38 | 0.662 | 0.043 | 0.663 | | HM vs DHA | 0.000 |
| | | HM | 56 | 1.475 | 0.081 | 1.333 | | HM vs DHA + ARA | 0.000 |
| | | | | | | | | Control vs HM | 0.000 |
| | | | | | | | | DHA vs DHA + ARA | 0.776 |

TABLE 10
Red Blood Cell Phosphatidylethanolamine Fatty Acids

| | | | | Arithmetic | Standard | | Regimen | Pairwise | |
|---|---|---|---|---|---|---|---|---|---|
| Time | Fatty Acid | Regimen | n | Mean | Error | Median | p-value | Comparison | Pairwise p-value |
| Study Form Initiation | 12:0 | Control | 52 | 0.069 | 0.015 | 0.022 | 0.546 | | |
| | | DHA | 57 | 0.075 | 0.013 | 0.033 | | | |
| | | DHA + ARA | 61 | 0.063 | 0.010 | 0.039 | | | |
| Study Form Initiation | 14:0 | Control | 52 | 0.307 | 0.038 | 0.220 | 0.792 | | |
| | | DHA | 57 | 0.278 | 0.025 | 0.206 | | | |
| | | DHA + ARA | 61 | 0.277 | 0.021 | 0.246 | | | |
| Study Form Initiation | 14:1 | Control | 52 | 0.080 | 0.015 | 0.032 | 0.181 | | |
| | | DHA | 57 | 0.061 | 0.012 | 0.028 | | | |
| | | DHA + ARA | 61 | 0.062 | 0.009 | 0.050 | | | |
| Study Form Initiation | 16:0 | Control | 52 | 20.021 | 0.736 | 17.945 | 0.967 | | |
| | | DHA | 57 | 19.847 | 0.622 | 19.295 | | | |
| | | DHA + ARA | 61 | 19.796 | 0.451 | 19.035 | | | |
| Study Form Initiation | 16:1 | Control | 52 | 0.731 | 0.035 | 0.698 | 0.337 | | |
| | | DHA | 57 | 0.769 | 0.034 | 0.746 | | | |
| | | DHA + ARA | 61 | 0.836 | 0.035 | 0.837 | | | |
| Study Form Initiation | 18:0 | Control | 52 | 8.857 | 0.329 | 8.469 | 0.142 | | |
| | | DHA | 57 | 8.434 | 0.227 | 8.308 | | | |
| | | DHA + ARA | 61 | 8.201 | 0.215 | 7.904 | | | |
| Study Form Initiation | 18:1 | Control | 52 | 16.450 | 0.301 | 16.698 | 0.412 | | |
| | | DHA | 57 | 16.208 | 0.326 | 16.308 | | | |
| | | DHA + ARA | 61 | 16.415 | 0.375 | 16.001 | | | |
| Study Form Initiation | 18:2 | Control | 52 | 6.615 | 0.253 | 6.682 | 0.773 | | |
| | | DHA | 57 | 6.336 | 0.280 | 6.346 | | | |
| | | DHA + ARA | 61 | 6.175 | 0.294 | 5.682 | | | |
| Study Form Initiation | 18:3n6 | Control | 52 | 0.165 | 0.018 | 0.145 | 0.040 | Control vs DHA | 0.373 |
| | | DHA | 57 | 0.190 | 0.019 | 0.152 | | Control vs DHA + ARA | 0.013 |
| | | DHA + ARA | 61 | 0.192 | 0.016 | 0.169 | | DHA vs DHA + ARA | 0.101 |
| Study Form Initiation | 20:0 | Control | 52 | 0.372 | 0.043 | 0.291 | 0.151 | | |
| | | DHA | 57 | 0.314 | 0.030 | 0.244 | | | |
| | | DHA + ARA | 61 | 0.259 | 0.024 | 0.186 | | | |
| Study Form Initiation | 18:3n3 | Control | 52 | 0.305 | 0.023 | 0.261 | 0.641 | | |
| | | DHA | 57 | 0.269 | 0.018 | 0.249 | | | |
| | | DHA + ARA | 61 | 0.257 | 0.016 | 0.225 | | | |
| Study Form Initiation | 20:1 | Control | 52 | 0.573 | 0.036 | 0.517 | 0.395 | | |
| | | DHA | 57 | 0.615 | 0.034 | 0.555 | | | |
| | | DHA + ARA | 61 | 0.571 | 0.027 | 0.544 | | | |
| Study Form Initiation | 18:4 | Control | 52 | 0.025 | 0.005 | 0.000 | 0.371 | | |
| | | DHA | 57 | 0.031 | 0.004 | 0.025 | | | |
| | | DHA + ARA | 61 | 0.030 | 0.007 | 0.021 | | | |
| Study Form Initiation | 20:2n6 | Control | 52 | 0.479 | 0.023 | 0.480 | 0.706 | | |
| | | DHA | 57 | 0.463 | 0.024 | 0.437 | | | |
| | | DHA + ARA | 61 | 0.443 | 0.028 | 0.427 | | | |
| Study Form Initiation | 20:3n6 | Control | 52 | 1.843 | 0.072 | 1.829 | 0.099 | | |
| | | DHA | 57 | 1.965 | 0.077 | 1.820 | | | |
| | | DHA + ARA | 61 | 1.973 | 0.064 | 1.911 | | | |
| Study Form Initiation | 20:4n6 | Control | 52 | 25.817 | 0.618 | 26.820 | 0.353 | | |
| | | DHA | 57 | 26.475 | 0.611 | 27.376 | | | |
| | | DHA + ARA | 61 | 26.747 | 0.645 | 27.708 | | | |
| Study Form Initiation | 22:1 | Control | 52 | 0.150 | 0.017 | 0.138 | 0.572 | | |
| | | DHA | 57 | 0.167 | 0.015 | 0.151 | | | |
| | | DHA + ARA | 61 | 0.168 | 0.017 | 0.141 | | | |
| Study Form Initiation | 20:5n3 | Control | 52 | 0.378 | 0.024 | 0.357 | 0.997 | | |
| | | DHA | 57 | 0.384 | 0.024 | 0.370 | | | |
| | | DHA + ARA | 61 | 0.366 | 0.022 | 0.335 | | | |
| Study Form Initiation | 22:4n6 | Control | 52 | 7.290 | 0.182 | 7.402 | 0.875 | | |
| | | DHA | 57 | 7.431 | 0.186 | 7.638 | | | |
| | | DHA + ARA | 61 | 7.456 | 0.167 | 7.270 | | | |
| Study Form Initiation | 24:1 | Control | 52 | 0.100 | 0.028 | 0.041 | 0.068 | | |
| | | DHA | 57 | 0.059 | 0.009 | 0.031 | | | |
| | | DHA + ARA | 61 | 0.072 | 0.010 | 0.047 | | | |
| Study Form Initiation | 22:5n6 | Control | 52 | 1.757 | 0.083 | 1.782 | 0.555 | | |
| | | DHA | 57 | 1.809 | 0.070 | 1.857 | | | |
| | | DHA + ARA | 61 | 1.851 | 0.075 | 1.775 | | | |
| Study Form Initiation | 22:4n3 | Control | 52 | 0.001 | 0.001 | 0.000 | 0.257 | | |
| | | DHA | 57 | 0.001 | 0.001 | 0.000 | | | |
| | | DHA + ARA | 61 | 0.005 | 0.002 | 0.000 | | | |
| Study Form Initiation | 22:5n3 | Control | 52 | 1.496 | 0.109 | 1.308 | 0.195 | | |
| | | DHA | 57 | 1.375 | 0.109 | 0.988 | | | |
| | | DHA + ARA | 61 | 1.380 | 0.097 | 1.041 | | | |
| Study Form Initiation | 22:6n3 | Control | 52 | 6.119 | 0.200 | 6.381 | 0.375 | | |
| | | DHA | 57 | 6.444 | 0.185 | 6.468 | | | |
| | | DHA + ARA | 61 | 6.407 | 0.220 | 6.579 | | | |
| Study Form Termination | 12:0 | Control | 53 | 0.093 | 0.018 | 0.033 | 0.630 | | |
| | | DHA | 55 | 0.093 | 0.019 | 0.036 | | | |
| | | DHA + ARA | 58 | 0.067 | 0.012 | 0.035 | | | |
| Study Form Termination | 14:0 | Control | 53 | 0.360 | 0.031 | 0.279 | 0.782 | | |
| | | DHA | 55 | 0.380 | 0.039 | 0.265 | | | |
| | | DHA + ARA | 58 | 0.348 | 0.030 | 0.256 | | | |
| Study Form Termination | 14:1 | Control | 53 | 0.086 | 0.020 | 0.041 | 0.592 | | |
| | | DHA | 55 | 0.066 | 0.013 | 0.000 | | | |
| | | DHA + ARA | 58 | 0.066 | 0.011 | 0.043 | | | |
| Study Form Termination | 16:0 | Control | 53 | 19.326 | 0.673 | 17.617 | 0.560 | | |
| | | DHA | 55 | 19.062 | 0.614 | 17.556 | | | |
| | | DHA + ARA | 58 | 18.357 | 0.467 | 17.568 | | | |
| Study Form Termination | 16:1 | Control | 53 | 0.511 | 0.034 | 0.476 | 0.604 | | |
| | | DHA | 55 | 0.579 | 0.045 | 0.509 | | | |
| | | DHA + ARA | 58 | 0.618 | 0.049 | 0.555 | | | |
| Study Form Termination | 18:0 | Control | 53 | 9.614 | 0.266 | 9.406 | 0.024 | Control vs DHA | 0.130 |
| | | DHA | 55 | 9.173 | 0.208 | 8.818 | | Control vs DHA + ARA | 0.006 |
| | | DHA + ARA | 58 | 8.961 | 0.242 | 8.697 | | DHA vs DHA + ARA | 0.219 |
| Study Form Termination | 18:1 | Control | 53 | 14.763 | 0.437 | 14.695 | 0.333 | | |
| | | DHA | 55 | 15.177 | 0.299 | 14.927 | | | |
| | | DHA + ARA | 58 | 14.814 | 0.330 | 14.499 | | | |
| Study Form Termination | 18:2 | Control | 53 | 9.405 | 0.192 | 9.359 | 0.000 | Control vs DHA | 0.908 |
| | | DHA | 55 | 9.180 | 0.207 | 9.188 | | Control vs DHA + ARA | 0.000 |
| | | DHA + ARA | 58 | 7.756 | 0.141 | 7.586 | | DHA vs DHA + ARA | 0.000 |
| Study Form Termination | 18:3n6 | Control | 53 | 0.169 | 0.012 | 0.163 | 0.160 | | |
| | | DHA | 55 | 0.187 | 0.017 | 0.157 | | | |
| | | DHA + ARA | 58 | 0.198 | 0.018 | 0.161 | | | |
| Study Form Termination | 20:0 | Control | 53 | 0.404 | 0.044 | 0.278 | 0.146 | | |
| | | DHA | 55 | 0.336 | 0.037 | 0.208 | | | |
| | | DHA + ARA | 58 | 0.288 | 0.029 | 0.208 | | | |
| Study Form Termination | 18:3n3 | Control | 53 | 0.382 | 0.017 | 0.364 | 0.134 | | |
| | | DHA | 55 | 0.368 | 0.016 | 0.354 | | | |
| | | DHA + ARA | 58 | 0.329 | 0.015 | 0.305 | | | |
| Study Form Termination | 20:1 | Control | 53 | 0.553 | 0.029 | 0.526 | 0.164 | | |
| | | DHA | 55 | 0.579 | 0.028 | 0.537 | | | |
| | | DHA + ARA | 58 | 0.507 | 0.025 | 0.483 | | | |
| Study Form Termination | 18:4 | Control | 53 | 0.042 | 0.010 | 0.018 | 0.108 | | |
| | | DHA | 55 | 0.026 | 0.005 | 0.019 | | | |
| | | DHA + ARA | 58 | 0.022 | 0.004 | 0.000 | | | |
| Study Form Termination | 20:2n6 | Control | 53 | 0.754 | 0.029 | 0.765 | 0.068 | | |
| | | DHA | 55 | 0.774 | 0.030 | 0.750 | | | |
| | | DHA + ARA | 58 | 0.654 | 0.026 | 0.663 | | | |
| Study Form Termination | 20:3n6 | Control | 53 | 2.253 | 0.111 | 2.073 | 0.203 | | |
| | | DHA | 55 | 2.295 | 0.094 | 2.206 | | | |
| | | DHA + ARA | 58 | 2.066 | 0.073 | 1.992 | | | |
| Study Form Termination | 20:4n6 | Control | 53 | 24.279 | 0.527 | 25.132 | 0.000 | Control vs DHA | 0.119 |
| | | DHA | 55 | 23.464 | 0.520 | 24.038 | | Control vs DHA + ARA | 0.000 |
| | | DHA + ARA | 58 | 26.760 | 0.437 | 27.372 | | DHA vs DHA + ARA | 0.000 |
| Study Form Termination | 22:1 | Control | 53 | 0.149 | 0.019 | 0.122 | 0.229 | | |
| | | DHA | 55 | 0.176 | 0.016 | 0.169 | | | |
| | | DHA + ARA | 58 | 0.146 | 0.012 | 0.130 | | | |
| Study Form Termination | 20:5n3 | Control | 53 | 0.519 | 0.020 | 0.493 | 0.000 | Control vs DHA | 0.286 |
| | | DHA | 55 | 0.563 | 0.025 | 0.575 | | Control vs DHA + ARA | 0.000 |
| | | DHA + ARA | 58 | 0.411 | 0.015 | 0.415 | | DHA vs DHA + ARA | 0.000 |
| Study Form Termination | 22:4n6 | Control | 53 | 7.309 | 0.208 | 7.656 | 0.007 | Control vs DHA | 0.025 |
| | | DHA | 55 | 7.135 | 0.154 | 6.885 | | Control vs DHA + ARA | 0.461 |
| | | DHA + ARA | 58 | 7.592 | 0.155 | 7.635 | | DHA vs DHA + ARA | 0.002 |
| Study Form Termination | 24:1 | Control | 53 | 0.092 | 0.023 | 0.038 | 0.294 | | |
| | | DHA | 55 | 0.056 | 0.009 | 0.042 | | | |
| | | DHA + ARA | 58 | 0.062 | 0.008 | 0.041 | | | |
| Study Form Termination | 22:5n6 | Control | 53 | 1.444 | 0.064 | 1.423 | 0.010 | Control vs DHA | 0.003 |
| | | DHA | 55 | 1.231 | 0.034 | 1.213 | | Control vs DHA + ARA | 0.255 |
| | | DHA + ARA | 58 | 1.347 | 0.040 | 1.330 | | DHA vs DHA + ARA | 0.050 |
| Study Form Termination | 22:4n3 | Control | 53 | 0.000 | 0.000 | 0.000 | 0.137 | | |
| | | DHA | 55 | 0.004 | 0.002 | 0.000 | | | |
| | | DHA + ARA | 58 | 0.004 | 0.002 | 0.000 | | | |
| Study Form Termination | 22:5n3 | Control | 53 | 2.694 | 0.110 | 2.839 | 0.003 | Control vs DHA | 0.004 |
| | | DHA | 55 | 2.334 | 0.091 | 2.400 | | Control vs DHA + ARA | 0.002 |
| | | DHA + ARA | 58 | 2.237 | 0.069 | 2.269 | | DHA vs DHA + ARA | 0.943 |
| Study Form Termination | 22:6n3 | Control | 53 | 4.798 | 0.151 | 4.815 | 0.000 | Control vs DHA | 0.000 |
| | | DHA | 55 | 6.762 | 0.183 | 7.043 | | Control vs DHA + ARA | 0.000 |
| | | DHA + ARA | 58 | 6.389 | 0.150 | 6.498 | | DHA vs DHA + ARA | 0.027 |
| 48 Weeks PCA | 12:0 | Control | 37 | 0.053 | 0.019 | 0.024 | 0.587 | | |
| | | DHA | 32 | 0.054 | 0.016 | 0.019 | | | |
| | | DHA + ARA | 38 | 0.047 | 0.014 | 0.018 | | | |
| | | HM | 56 | 0.045 | 0.011 | 0.023 | | | |
| 48 Weeks PCA | 14:0 | Control | 37 | 0.243 | 0.030 | 0.169 | 0.598 | | |
| | | DHA | 32 | 0.251 | 0.041 | 0.162 | | | |
| | | DHA + ARA | 38 | 0.235 | 0.025 | 0.188 | | | |
| | | HM | 56 | 0.230 | 0.016 | 0.210 | | | |
| 48 Weeks PCA | 14:1 | Control | 37 | 0.080 | 0.017 | 0.037 | 0.092 | | |
| | | DHA | 32 | 0.055 | 0.017 | 0.000 | | | |
| | | DHA + ARA | 38 | 0.078 | 0.019 | 0.044 | | | |
| | | HM | 56 | 0.053 | 0.011 | 0.021 | | | |
| 48 Weeks PCA | 16:0 | Control | 37 | 17.319 | 0.595 | 16.314 | 0.177 | | |
| | | DHA | 32 | 17.101 | 0.729 | 15.692 | | | |
| | | DHA + ARA | 38 | 17.225 | 0.538 | 16.997 | | | |
| | | HM | 56 | 18.138 | 0.395 | 17.607 | | | |
| 48 Weeks PCA | 16:1 | Control | 37 | 0.440 | 0.050 | 0.349 | 0.000 | Control vs DHA | 0.601 |
| | | DHA | 32 | 0.390 | 0.035 | 0.336 | | Control vs DHA + ARA | 0.524 |
| | | DHA + ARA | 38 | 0.390 | 0.022 | 0.376 | | HM vs DHA | 0.000 |
| | | HM | 56 | 0.596 | 0.027 | 0.562 | | HM vs DHA + ARA | 0.000 |
| | | | | | | | | Control vs HM | 0.001 |
| | | | | | | | | DHA vs DHA + ARA | 0.928 |
| 48 Weeks PCA | 18:0 | Control | 37 | 7.935 | 0.327 | 7.174 | 0.000 | Control vs DHA | 0.347 |
| | | DHA | 32 | 7.962 | 0.293 | 7.552 | | Control vs DHA + ARA | 0.483 |
| | | DHA + ARA | 38 | 7.443 | 0.270 | 7.173 | | HM vs DHA | 0.020 |
| | | HM | 56 | 8.754 | 0.230 | 8.409 | | HM vs DHA + ARA | 0.000 |
| | | | | | | | | Control vs HM | 0.001 |
| | | | | | | | | DHA vs DHA + ARA | 0.108 |
| 48 Weeks PCA | 18:1 | Control | 37 | 19.438 | 0.368 | 19.410 | 0.038 | Control vs DHA | 0.401 |
| | | DHA | 32 | 19.066 | 0.421 | 19.534 | | Control vs DHA + ARA | 0.234 |
| | | DHA + ARA | 38 | 19.302 | 0.332 | 19.433 | | HM vs DHA | 0.067 |
| | | HM | 56 | 18.469 | 0.278 | 18.141 | | HM vs DHA + ARA | 0.118 |
| | | | | | | | | Control vs HM | 0.005 |
| | | | | | | | | DHA vs DHA + ARA | 0.758 |
| 48 Weeks PCA | 18:2 | Control | 37 | 9.328 | 0.261 | 9.267 | 0.000 | Control vs DHA | 0.024 |
| | | DHA | 32 | 8.867 | 0.210 | 8.696 | | Control vs DHA + ARA | 0.187 |
| | | DHA + ARA | 38 | 9.257 | 0.216 | 8.840 | | HM vs DHA | 0.000 |
| | | HM | 56 | 6.291 | 0.193 | 6.027 | | HM vs DHA + ARA | 0.000 |
| | | | | | | | | Control vs HM | 0.000 |
| | | | | | | | | DHA vs DHA + ARA | 0.318 |
| 48 Weeks PCA | 18:3n6 | Control | 37 | 0.198 | 0.020 | 0.182 | 0.050 | Control vs DHA | 0.879 |
| | | DHA | 32 | 0.219 | 0.031 | 0.171 | | Control vs DHA + ARA | 0.590 |
| | | DHA + ARA | 38 | 0.188 | 0.021 | 0.158 | | HM vs DHA | 0.029 |
| | | HM | 56 | 0.129 | 0.012 | 0.112 | | HM vs DHA + ARA | 0.061 |
| | | | | | | | | Control vs HM | 0.014 |
| | | | | | | | | DHA vs DHA + ARA | 0.714 |
| 48 Weeks PCA | 20:0 | Control | 37 | 0.263 | 0.058 | 0.146 | 0.728 | | |
| | | DHA | 32 | 0.262 | 0.042 | 0.145 | | | |
| | | DHA + ARA | 38 | 0.212 | 0.037 | 0.125 | | | |
| | | HM | 56 | 0.295 | 0.031 | 0.240 | | | |
| 48 Weeks PCA | 18:3n3 | Control | 37 | 0.291 | 0.025 | 0.225 | 0.001 | Control vs DHA | 0.559 |
| | | DHA | 32 | 0.270 | 0.017 | 0.262 | | Control vs DHA + ARA | 0.848 |
| | | DHA + ARA | 38 | 0.265 | 0.015 | 0.245 | | HM vs DHA | 0.008 |
| | | HM | 56 | 0.226 | 0.020 | 0.169 | | HM vs DHA + ARA | 0.002 |
| | | | | | | | | Control vs HM | 0.001 |
| | | | | | | | | DHA vs DHA + ARA | 0.689 |
| 48 Weeks PCA | 20:1 | Control | 37 | 0.715 | 0.031 | 0.648 | 0.000 | Control vs DHA | 0.339 |
| | | DHA | 32 | 0.772 | 0.032 | 0.782 | | Control vs DHA + ARA | 0.512 |
| | | DHA + ARA | 38 | 0.936 | 0.188 | 0.738 | | HM vs DHA | 0.000 |
| | | HM | 56 | 0.533 | 0.024 | 0.492 | | HM vs DHA + ARA | 0.000 |
| | | | | | | | | Control vs HM | 0.000 |
| | | | | | | | | DHA vs DHA + ARA | 0.115 |
| 48 Weeks PCA | 18:4 | Control | 37 | 0.017 | 0.005 | 0.003 | 0.057 | | |
| | | DHA | 32 | 0.017 | 0.005 | 0.000 | | | |
| | | DHA + ARA | 38 | 0.023 | 0.006 | 0.000 | | | |
| | | HM | 56 | 0.027 | 0.004 | 0.019 | | | |
| 48 Weeks PCA | 20:2n6 | Control | 37 | 0.672 | 0.035 | 0.698 | 0.000 | Control vs DHA | 0.543 |
| | | DHA | 32 | 0.668 | 0.026 | 0.684 | | Control vs DHA + ARA | 0.532 |
| | | DHA + ARA | 38 | 0.715 | 0.032 | 0.689 | | HM vs DHA | 0.000 |
| | | HM | 56 | 0.444 | 0.016 | 0.412 | | HM vs DHA + ARA | 0.000 |
| | | | | | | | | Control vs HM | 0.000 |
| | | | | | | | | DHA vs DHA + ARA | 0.995 |
| 48 Weeks PCA | 20:3n6 | Control | 37 | 2.138 | 0.099 | 1.999 | 0.012 | Control vs DHA | 0.896 |
| | | DHA | 32 | 2.165 | 0.100 | 2.045 | | Control vs DHA + ARA | 0.935 |
| | | DHA + ARA | 38 | 2.172 | 0.114 | 2.132 | | HM vs DHA | 0.015 |
| | | HM | 56 | 1.715 | 0.053 | 1.637 | | HM vs DHA + ARA | 0.006 |
| | | | | | | | | Control vs HM | 0.007 |
| | | | | | | | | DHA vs DHA + ARA | 0.835 |
| 48 Weeks PCA | 20:4n6 | Control | 37 | 24.508 | 0.536 | 24.774 | 0.950 | | |
| | | DHA | 32 | 24.428 | 0.491 | 25.206 | | | |
| | | DHA + ARA | 38 | 24.788 | 0.429 | 25.122 | | | |
| | | HM | 56 | 24.625 | 0.384 | 25.189 | | | |
| 48 Weeks PCA | 22:1 | Control | 37 | 0.168 | 0.016 | 0.172 | 0.121 | | |
| | | DHA | 32 | 0.189 | 0.022 | 0.188 | | | |
| | | DHA + ARA | 38 | 0.154 | 0.022 | 0.133 | | | |
| | | HM | 56 | 0.148 | 0.013 | 0.134 | | | |
| 48 Weeks PCA | 20:5n3 | Control | 37 | 0.382 | 0.026 | 0.368 | 0.497 | | |
| | | DHA | 32 | 0.369 | 0.015 | 0.377 | | | |
| | | DHA + ARA | 38 | 0.347 | 0.011 | 0.347 | | | |
| | | HM | 56 | 0.384 | 0.016 | 0.360 | | | |
| 48 Weeks PCA | 22:4n6 | Control | 37 | 8.580 | 0.267 | 8.761 | 0.001 | Control vs DHA | 0.612 |
| | | DHA | 32 | 8.791 | 0.250 | 9.132 | | Control vs DHA + ARA | 0.416 |
| | | DHA + ARA | 38 | 8.576 | 0.188 | 8.472 | | HM vs DHA | 0.000 |
| | | HM | 56 | 7.727 | 0.203 | 7.618 | | HM vs DHA + ARA | 0.013 |
| | | | | | | | | Control vs HM | 0.001 |
| | | | | | | | | DHA vs DHA + ARA | 0.199 |
| 48 Weeks PCA | 24:1 | Control | 37 | 0.067 | 0.016 | 0.035 | 0.943 | | |
| | | DHA | 32 | 0.049 | 0.009 | 0.034 | | | |
| | | DHA + ARA | 38 | 0.046 | 0.008 | 0.036 | | | |
| | | HM | 56 | 0.062 | 0.016 | 0.027 | | | |
| 48 Weeks PCA | 22:5n6 | Control | 37 | 1.401 | 0.066 | 1.411 | 0.000 | Control vs DHA | 0.977 |
| | | DHA | 32 | 1.353 | 0.057 | 1.414 | | Control vs DHA + ARA | 0.997 |
| | | DHA + ARA | 38 | 1.364 | 0.054 | 1.359 | | HM vs DHA | 0.000 |
| | | HM | 56 | 1.883 | 0.056 | 1.889 | | HM vs DHA + ARA | 0.000 |
| | | | | | | | | Control vs HM | 0.000 |
| | | | | | | | | DHA vs DHA + ARA | 0.975 |
| 48 Weeks PCA | 22:4n3 | Control | 37 | 0.000 | 0.000 | 0.000 | 1.000 | | |
| | | DHA | 32 | 0.000 | 0.000 | 0.000 | | | |
| | | DHA + ARA | 38 | 0.000 | 0.000 | 0.000 | | | |
| | | HM | 56 | 0.001 | 0.001 | 0.000 | | | |
| 48 Weeks PCA | 22:5n3 | Control | 37 | 2.567 | 0.092 | 2.681 | 0.000 | Control vs DHA | 0.884 |
| | | DHA | 32 | 2.561 | 0.086 | 2.630 | | Control vs DHA + ARA | 0.148 |
| | | DHA + ARA | 38 | 2.436 | 0.066 | 2.443 | | HM vs DHA | 0.000 |
| | | HM | 56 | 1.942 | 0.065 | 1.978 | | HM vs DHA + ARA | 0.000 |
| | | | | | | | | Control vs HM | 0.000 |
| | | | | | | | | DHA vs DHA + ARA | 0.213 |
| 48 Weeks PCA | 22:6n3 | Control | 37 | 3.196 | 0.159 | 3.013 | 0.000 | Control vs DHA | 0.000 |
| | | DHA | 32 | 4.143 | 0.177 | 4.079 | | Control vs DHA + ARA | 0.000 |
| | | DHA + ARA | 38 | 3.801 | 0.134 | 3.721 | | HM vs DHA | 0.000 |
| | | HM | 56 | 7.283 | 0.201 | 7.341 | | HM vs DHA + ARA | 0.000 |
| | | | | | | | | Control vs HM | 0.000 |
| | | | | | | | | DHA vs DHA + ARA | 0.281 |

TABLE 11
Preterm Infant Complications

| | Regimen | | | |
|---|---|---|---|---|
| | Control | DHA | DHA + ARA | p-value* |
| Retinopathy of | | | | |
| Prematurity Test | | | | |
| Results | | | | |
| Absent | 34 (76%) | 44 (76%) | 41 (79%) | 0.91 |
| I | 8 (18%) | 11 (19%) | 6 (12%) | |
| II | 2 (4%) | 2 (3%) | 4 (8%) | |
| III | 1 (2%) | 1 (2%) | | |
| Present, but not | | | 1 (2%) | |
| graded | | | | |
| Ultrasound | | | | |
| Examination for | | | | |
| Intraventricular | | | | |
| Hemorrhage | | | | |
| None | 47 (81%) | 52 (84%) | 49 (80%) | 0.78 |
| Stage 1 | 6 (10%) | 9 (15%) | 7 (11%) | |
| Stage 2 | 3 (5%) | | 2 (3%) | |
| Stage 3 | 1 (2%) | | 1 (2%) | |
| Stage 4 | 1 (2%) | | 2 (3%) | |
| Questionable | | 1 (2%) | | |
| Posthemorrhagic | | | | |
| Hydrocephalus | | | | |
| developed? | | | | |
| No | 61 (98%) | 65 (98%) | 64 (97%) | 1.00 |
| Yes | 1 (2%) | 1 (2%) | 2 (3%) | |

*The statistical test was based on a dichotomous response: present or absent.

TABLE 12
Serious Adverse Events Reported During Study Formula Phase

| | Regimen | | | p- |
|---|---|---|---|---|
| Event | Control | DHA | DHA + ARA | value |
| Any Event | 4 (6%) | 3 (5%) | 4 (6%) | 0.93 |
| Other Respiratory Conditions | 2 (3%) | 0 | 0 | 0.10 |
| of Fetus and Newborn | | | | |
| Other Infection Specific to | 1 (2%) | 0 | 0 | 0.32 |
| the Perinatal Period | | | | |
| Intraventricular Hemorrhage | 0 | 0 | 1 (2%) | 1.00 |
| Other Specified Perinatal | 0 | 1 (2%) | 0 | 1.00 |
| Disorders of Digestive System | | | | |
| Convulsions in Newborn | 1 (2%) | 0 | 0 | 0.32 |
| Feeding Problems in Newborn | 0 | 1 (2%) | 1 (2%) | 1.00 |
| Hernia | 0 | 0 | 1 (2%) | 1.00 |
| Other | 0 | 1 (2%) | 1 (2%) | 1.00 |

TABLE 13
Serious Adverse Events Reported During the Term Formula Phase

| | Regimen | | | | | |
|---|---|---|---|---|---|---|
| Event | Control | DHA | DHA + ARA | HM | p-value | |
| Any Event | 7 (13%) | 9 (15%) | 9 (15%) | 1 (1%) | 0.002 | |
| | | | | | C vs D | 0.79 |
| | | | | | C vs D + A | 0.79 |
| | | | | | D vs D + A | 1.00 |
| | | | | | C vs HM | 0.006 |
| | | | | | D vs HM | 0.001 |
| | | | | | D + A vs HM | 0.001 |
| Infectious Colitis, | 0 | 0 | 1 (2%) | 0 | 0.67 | |
| Enteritis, and | | | | | | |
| Gastroenteritis | | | | | | |
| Croup | 0 | 0 | 1 (2%) | 0 | 0.67 | |
| Bronchopneumonia, | 2 (4%) | 3 (5%) | 6 (10%) | 0 | 0.013 | |
| Organism Unspecified | | | | | C vs D | 1.00 |
| | | | | | C vs D + A | 0.27 |
| | | | | | D vs D + A | 0.49 |
| | | | | | C vs HM | 0.15 |
| | | | | | D vs HM | 0.064 |
| | | | | | D + A vs HM | 0.004 |

| | | | | | |
|---|---|---|---|---|---|
| Asthma, Unspecified | 1 (2%) | 0 | 0 | 0 | 0.21 |
| Esophageal Reflux | 0 | 1 (2%) | 2 (3%) | 0 | 0.23 |
| Dyspepsia and Other | 0 | 0 | 0 | 1 (1%) | 1.0 |
| Stomach Function | | | | | |
| Disorder | | | | | |
| Other Respiratory | 1 (2%) | 1 (2%) | 3 (5%) | 0 | 0.11 |
| Conditions of Fetus | | | | | |
| and Newborn | | | | | |
| Convulsions | 1 (2%) | 0 | 0 | 0 | 0.21 |
| Sudden Infant Death | 1 (2%) | 1 (2%) | 0 | 0 | 0.34 |
| Syndrome | | | | | |
| Hernia | 2 (4%) | 2 (3%) | 0 | 0 | 0.11 |
| Other | 0 | 3 (5%) | 2 (3%) | 0 | 0.063 |

APPENDIX 1
Listing of Weights Included in the Statistical Analyses

| Gender | Regimen | Subject | Variable | Wgt1 | Wgt2 | Wgt3 | Wgt4 | Wgt5 | Wgt6 | Wgt7 | Wgt8 | Wgt9 | Growth Rate g/day | Wgt_40 | Wgt_48 | Wgt_57 |
|---|---|---|---|---|---|---|---|---|---|---|---|---|---|---|---|---|
| Male | Control | 9698-0301 | Weight (g) | 1120 | 1240 | 1360 | 1590 | 1870 | | | | | 27.7 | | | |
| | | | Age (weeks pca) | 30.3 | 31.3 | 32.1 | 33.1 | 34.1 | | | | | | | | |
| Male | Control | 9698-0304 | Weight (g) | 1450 | 1630 | 1940 | 2180 | | | | | | 36.1 | 3731 | 5752 | 6816 |
| | | | Age (weeks pca) | 32.6 | 33.4 | 34.7 | 35.4 | | | | | | | 40.3 | 48.3 | 56.6 |
| Male | Control | 9699-0302 | Weight (g) | 958.0 | 1108 | 1251 | 1378 | 1659 | | | | | 23.9 | 3064 | 4993 | 6553 |
| | | | Age (weeks pca) | 30.7 | 31.7 | 32.7 | 33.7 | 34.7 | | | | | | 39.9 | 48.0 | 57.9 |
| Male | Control | 9699-0306 | Weight (g) | 1185 | 1261 | 1437 | 1647 | 1933 | | | | | 26.9 | 3575 | 4936 | 6014 |
| | | | Age (weeks pca) | 31.0 | 32.0 | 33.0 | 34.0 | 35.0 | | | | | | 40.3 | 48.3 | 57.1 |
| Male | Control | 9699-0308 | Weight (g) | 1600 | 1840 | 2752 | | | | | | | 43.3 | 3688 | 5504 | 6922 |
| | | | Age (weeks pca) | 34.4 | 35.4 | 38.3 | | | | | | | | 40.3 | 48.3 | 57.3 |
| Male | Control | 9700-0301 | Weight (g) | 1810 | 1855 | 2075 | 2330 | 2595 | 3120 | | | | 36.2 | 3745 | 5080 | 6610 |
| | | | Age (weeks pca) | 32.1 | 32.6 | 33.4 | 34.4 | 35.4 | 37.4 | | | | | 40.1 | 47.6 | 56.7 |
| Male | Control | 9701-0303 | Weight (g) | 1181 | 1298 | 1494 | 1785 | 2012 | | | | | 31.5 | 3070 | 3895 | 4965 |
| | | | Age (weeks pca) | 32.4 | 33.4 | 34.4 | 35.4 | 36.3 | | | | | | 41.6 | 48.6 | 57.6 |
| Male | Control | 9701-0304 | Weight (g) | 1412 | 1566 | 1851 | 2117 | 2318 | | | | | 34.1 | 3070 | 5445 | 7135 |
| | | | Age (weeks pca) | 31.9 | 32.9 | 33.7 | 34.7 | 35.9 | | | | | | 39.9 | 48.3 | 56.9 |
| Male | Control | 9702-0302 | Weight (g) | 1480 | 1775 | 2045 | 2240 | 2340 | 2570 | | | | 33.8 | 3590 | 4840 | 6110 |
| | | | Age (weeks pca) | 31.0 | 32.1 | 33.0 | 34.0 | 34.6 | 35.6 | | | | | 40.1 | 48.6 | 58.4 |
| Male | Control | 9703-0302 | Weight (g) | 1785 | 2040 | 2375 | 2685 | 2955 | | | | | 41.7 | 3620 | 5850 | 7470 |
| | | | Age (weeks pca) | 33.3 | 34.6 | 35.6 | 36.4 | 37.4 | | | | | | 39.7 | 48.6 | 57.3 |
| Male | Control | 9703-0304 | Weight (g) | 1475 | 1705 | 1920 | 2190 | 2425 | | | | | 34.2 | 3170 | 5240 | 6970 |
| | | | Age (weeks pca) | 31.7 | 33.0 | 34.0 | 34.9 | 35.7 | | | | | | 40.1 | 47.7 | 57.1 |
| Male | Control | 9703-0308 | Weight (g) | 1140 | 1230 | 1445 | 1665 | 1945 | | | | | 28.9 | 2520 | 4010 | 5030 |
| | | | Age (weeks pca) | 31.7 | 32.6 | 33.7 | 34.7 | 35.7 | | | | | | 39.7 | 48.4 | 56.9 |
| Male | Control | 9704-0303 | Weight (g) | 975.0 | 1205 | 1270 | 1450 | 1665 | 1760 | 2045 | | | 24.4 | 2150 | 3700 | 4950 |
| | | | Age (weeks pca) | 32.3 | 33.4 | 34.4 | 35.4 | 36.3 | 37.3 | 38.3 | | | | 39.3 | 48.3 | 57.4 |
| Male | Control | 9704-0305 | Weight (g) | 1315 | 1475 | 1640 | 1860 | | | | | | 23.7 | | | |
| | | | Age (weeks pca) | 30.9 | 32.0 | 33.0 | 34.1 | | | | | | | | | |
| Male | Control | 9705-0302 | Weight (g) | 1280 | 1389 | 1588 | 1786 | 2240 | | | | | 30.9 | 2540 | 4936 | 5646 |
| | | | Age (weeks pca) | 33.0 | 34.0 | 35.0 | 36.0 | 37.4 | | | | | | 39.6 | 47.4 | 56.4 |
| Male | Control | 9705-0304 | Weight (g) | 1270 | 1280 | 1570 | 1810 | | | | | | 25.3 | 3291 | 5816 | 7490 |
| | | | Age (weeks pca) | 31.3 | 32.3 | 33.3 | 34.6 | | | | | | | 39.7 | 47.7 | 56.7 |
| Male | Control | 9706-0302 | Weight (g) | 1645 | 1865 | 2130 | 2435 | | | | | | 37.1 | 2800 | 4660 | 6170 |
| | | | Age (weeks pca) | 35.7 | 36.6 | 37.7 | 38.7 | | | | | | | 40.1 | 48.7 | 56.7 |
| Male | Control | 9706-0303 | Weight (g) | 1875 | 1984 | 2135 | 2185 | 2465 | | | | | 22.2 | 3050 | 4550 | 6675 |
| | | | Age (weeks pca) | 33.7 | 34.7 | 35.6 | 36.4 | 37.3 | | | | | | 41.0 | 48.6 | 56.9 |
| Male | Control | 9706-0308 | Weight (g) | 1655 | 1734 | 2005 | 2495 | | | | | | 46.9 | 3835 | 5155 | 6090 |
| | | | Age (weeks pca) | 32.9 | 33.1 | 34.0 | 35.4 | | | | | | | 40.6 | 48.0 | 56.3 |
| Male | Control | 9707-0302 | Weight (g) | 1544 | 1820 | 2215 | 2450 | 2460 | | | | | 32.8 | 2930 | 3795 | 5185 |
| | | | Age (weeks pca) | 31.6 | 32.9 | 34.4 | 35.4 | 35.7 | | | | | | 40.1 | 47.7 | 56.6 |
| Male | Control | 9707-0303 | Weight (g) | 1415 | 1600 | 1850 | 2195 | 2310 | | | | | 32.7 | 2530 | 4235 | 6530 |
| | | | Age (weeks pca) | 33.1 | 34.1 | 35.1 | 36.6 | 37.1 | | | | | | 39.7 | 47.7 | 57.1 |
| Male | Control | 9707-0309 | Weight (g) | 1046 | 1442 | 1644 | 1910 | | | | | | 30.7 | 2965 | 4465 | |
| | | | Age (weeks pca) | 30.9 | 32.7 | 33.7 | 34.9 | | | | | | | 39.9 | 48.0 | |
| Male | Control | 9708-0303 | Weight (g) | 1730 | 1960 | 2205 | 2520 | | | | | | 37.4 | 3680 | 5470 | 7330 |
| | | | Age (weeks pca) | 32.7 | 33.7 | 34.7 | 35.7 | | | | | | | 40.1 | 48.1 | 57.0 |
| Male | Control | 9709-0302 | Weight (g) | 1090 | 1440 | 1660 | 1910 | 2040 | | | | | 30.8 | 3845 | 5700 | 6775 |
| | | | Age (weeks pca) | 29.9 | 31.7 | 32.7 | 33.7 | 34.3 | | | | | | 39.9 | 48.0 | 56.7 |
| Male | Control | 9712-0301* | Weight (g) | 1245 | 1221 | 1245 | 1291 | 1294 | 1330 | 1369 | 1402 | 1433 | 26.1 | | | |
| | | | Age (weeks pca) | 31.6 | 31.7 | 31.9 | 32.0 | 32.1 | 32.3 | 32.4 | 32.6 | 32.7 | | | | |
| Male | Control | 9712-0302 | Weight (g) | 1292 | 1345 | 1456 | 1670 | 1835 | 1985 | | | | 21.0 | 2160 | 3300 | 3980 |
| | | | Age (weeks pca) | 33.1 | 34.1 | 35.1 | 36.1 | 37.1 | 38.1 | | | | | 40.1 | 47.7 | 57.3 |
| Male | Control | 9743-0301 | Weight (g) | 1520 | 1570 | 1670 | 1720 | | | | | | 10.0 | 2260 | 4535 | |
| | | | Age (weeks pca) | 34.1 | 35.0 | 36.0 | 37.1 | | | | | | | 41.0 | 50.0 | |
| Male | Control | 9746-0301 | Weight (g) | 2065 | 2465 | 2760 | 3085 | 3085 | | | | | 48.9 | 3085 | 4795 | 6695 |
| | | | Age (weeks pca) | 37.6 | 38.9 | 39.7 | 40.6 | 40.6 | | | | | | 40.6 | 47.6 | 57.6 |
| Male | DHA | 9698-0302 | Weight (g) | 1640 | 1860 | 3170 | | | | | | | 47.5 | 3170 | 5206 | 7036 |
| | | | Age (weeks pca) | 35.1 | 36.1 | 39.9 | | | | | | | | 39.9 | 47.9 | 57.1 |
| Male | DHA | 9698-0306 | Weight (g) | 1620 | 1830 | 2090 | 2575 | | | | | | 28.3 | 2575 | 4334 | 6022 |
| | | | Age (weeks pca) | 35.1 | 36.3 | 37.3 | 40.0 | | | | | | | 40.0 | 48.0 | 57.0 |
| Male | DHA | 9699-0301 | Weight (g) | 1018 | 1207 | 1360 | 1617 | | | | | | 27.9 | 3121 | 5192 | 6752 |
| | | | Age (weeks pca) | 31.3 | 32.3 | 33.3 | 34.3 | | | | | | | 39.9 | 48.0 | 57.9 |
| Male | DHA | 9699-0303 | Weight (g) | 1258 | 1435 | 1631 | 1882 | 2724 | | | | | 48.3 | 2724 | 4341 | 5674 |
| | | | Age (weeks pca) | 32.4 | 33.4 | 34.4 | 35.4 | 36.4 | | | | | | 40.1 | 48.1 | 57.0 |
| Male | DHA | 9699-0307 | Weight (g) | 1182 | 1358 | 1484 | 1666 | | | | | | 22.5 | 1986 | 3206 | 4511 |
| | | | Age (weeks pca) | 34.7 | 35.7 | 36.7 | 37.7 | | | | | | | 40.0 | 48.0 | 57.0 |
| Male | DHA | 9700-0303 | Weight (g) | 1830 | 1980 | 2450 | 3045 | | | | | | 45.4 | 3585 | 5420 | 7035 |
| | | | Age (weeks pca) | 33.9 | 34.4 | 35.9 | 37.7 | | | | | | | 39.6 | 47.4 | 56.7 |
| Male | DHA | 9701-0301 | Weight (g) | 1098 | 1234 | 1365 | 1689 | 1902 | 2019 | 2104 | 2276 | 2288 | 20.4 | 2805 | 3405 | 4660 |
| | | | Age (weeks pca) | 29.6 | 30.6 | 31.6 | 33.4 | 34.6 | 35.6 | 36.4 | 37.4 | 38.6 | | 40.4 | 47.6 | 57.0 |
| Male | DHA | 9701-0305 | Weight (g) | 1621 | 1829 | 1880 | 2253 | 2582 | | | | | 34.7 | 3660 | | |
| | | | Age (weeks pca) | 31.7 | 33.1 | 33.7 | 34.7 | 35.7 | | | | | | 39.7 | | |
| Male | DHA | 9703-0303 | Weight (g) | 1775 | 2030 | 2285 | 2595 | 2780 | | | | | 38.2 | 3080 | 3940 | 5260 |
| | | | Age (weeks pca) | 33.3 | 34.1 | 35.1 | 36.0 | 37.1 | | | | | | 39.9 | 48.0 | 56.9 |
| Male | DHA | 9703-0306 | Weight (g) | 1725 | 1870 | 2180 | | | | | | | 41.7 | | | |
| | | | Age (weeks pca) | 33.4 | 34.0 | 35.0 | | | | | | | | | | |
| Male | DHA | 9703-0307 | Weight (g) | 1525 | 1725 | 2020 | 2390 | | | | | | 37.6 | 3120 | 4410 | 5600 |
| | | | Age (weeks pca) | 32.7 | 33.7 | 34.9 | 36.0 | | | | | | | 40.7 | 47.9 | 56.9 |
| Male | DHA | 9704-0304 | Weight (g) | 1380 | 1570 | 1730 | 1960 | 2140 | | | | | 29.3 | 2880 | 3900 | 4300 |
| | | | Age (weeks pca) | 32.1 | 33.1 | 34.1 | 35.0 | 35.9 | | | | | | 40.3 | 48.3 | 57.3 |
| Male | DHA | 9704-0306 | Weight (g) | 1320 | 1370 | 1550 | 1760 | 2020 | 2170 | | | | 25.6 | | 3750 | 4800 |
| | | | Age (weeks pca) | 30.7 | 31.7 | 32.7 | 33.7 | 34.7 | 35.9 | | | | | | 48.0 | 57.0 |
| Male | DHA | 9705-0303 | Weight (g) | 1380 | 1446 | 1616 | 1843 | 2330 | | | | | 30.8 | 2370 | 4170 | 5787 |
| | | | Age (weeks pca) | 33.0 | 34.0 | 35.0 | 36.0 | 37.4 | | | | | | 39.6 | 47.4 | 56.4 |
| Male | DHA | 9705-0305 | Weight (g) | 1490 | 1770 | 1980 | 2240 | | | | | | 36.7 | 3291 | | |
| | | | Age (weeks pca) | 31.1 | 32.1 | 33.1 | 34.0 | | | | | | | 39.6 | | |
| Male | DHA | 9706-0304 | Weight (g) | 1490 | 1655 | 1915 | 2260 | | | | | | 36.8 | 3335 | 5265 | 6900 |
| | | | Age (weeks pca) | 33.0 | 33.7 | 34.7 | 36.0 | | | | | | | 40.0 | 48.1 | 57.3 |
| Male | DHA | 9706-0306 | Weight (g) | 1604 | 1908 | 2160 | | | | | | | 42.8 | 3310 | 4205 | 5600 |
| | | | Age (weeks pca) | 34.4 | 35.4 | 36.3 | | | | | | | | 41.4 | 47.6 | 56.9 |
| Male | DHA | 9707-0001 | Weight (g) | 1305 | 1429 | | | | | | | | 17.7 | | | |
| | | | Age (weeks pca) | 31.0 | 32.0 | | | | | | | | | | | |
| Male | DHA | 9707-0304 | Weight (g) | 1555 | 1740 | 1990 | 2400 | 2570 | | | | | 36.9 | 3280 | 5115 | 6755 |
| | | | Age (weeks pca) | 32.0 | 33.0 | 34.0 | 35.4 | 36.0 | | | | | | 39.9 | 48.0 | 57.6 |
| Male | DHA | 9707-0306 | Weight (g) | 1728 | 2040 | 2260 | 3050 | 3050 | | | | | 43.2 | 3050 | 5100 | 7150 |
| | | | Age (weeks pca) | 36.1 | 37.3 | 38.1 | 40.6 | 40.6 | | | | | | 40.6 | 48.6 | 57.6 |
| Male | DHA | 9707-0307* | Weight (g) | 1649 | 1675 | 1699 | 1732 | 1778 | 1811 | 1858 | 1882 | 1938 | 39.6 | | | |
| | | | Age (weeks pca) | 32.4 | 32.6 | 32.7 | 32.9 | 33.0 | 33.1 | 33.3 | 33.4 | 33.6 | | | | |
| Male | DHA | 9707-1308 | Weight (g) | 1780 | 2045 | 3004 | 3004 | | | | | | 36.7 | 3004 | 4420 | 6090 |
| | | | Age (weeks pca) | 34.4 | 35.7 | 39.3 | 39.3 | | | | | | | 39.3 | 47.3 | 57.7 |
| Male | DHA | 9707-2308 | Weight (g) | 1651 | 1923 | 2850 | 2850 | | | | | | 35.8 | 2850 | 4375 | 5930 |
| | | | Age (weeks pca) | 34.4 | 35.7 | 39.3 | 39.3 | | | | | | | 39.3 | 47.3 | 57.7 |
| Male | DHA | 9708-0302 | Weight (g) | 1485 | 1740 | 2500 | | | | | | | 39.2 | 3873 | | 6256 |
| | | | Age (weeks pca) | 33.3 | 34.3 | 37.0 | | | | | | | | 42.9 | | 57.3 |
| Male | DHA | 9709-0301 | Weight (g) | 1490 | 1740 | 2000 | 2400 | 2800 | | | | | 44.4 | 3150 | 5080 | 6750 |
| | | | Age (weeks pca) | 32.4 | 33.4 | 34.4 | 35.4 | 36.7 | | | | | | 39.4 | 47.4 | 56.4 |
| Male | DHA | 9709-0304 | Weight (g) | 1470 | 1520 | | | | | | | | 7.1 | | | |
| | | | Age (weeks pca) | 34.4 | 35.4 | | | | | | | | | | | |
| Male | DHA | 9712-0304 | Weight (g) | 1545 | 1800 | 1985 | 2160 | 2550 | | | | | 30.5 | 3160 | 5200 | 7300 |
| | | | Age (weeks pca) | 33.0 | 34.0 | 35.0 | 36.0 | 37.6 | | | | | | 40.3 | 48.1 | 57.1 |
| Male | DHA | 9712-0306 | Weight (g) | 1240 | 1435 | 1695 | 1945 | | | | | | 33.9 | 3040 | 4680 | 5860 |
| | | | Age (weeks pca) | 31.5 | 32.5 | 33.5 | 34.5 | | | | | | | 39.6 | 48.6 | 57.6 |
| Male | DHA | 9743-0303 | Weight (g) | 1700 | 1810 | 2100 | 2300 | | | | | | 31.1 | 3100 | 5500 | |
| | | | Age (weeks pca) | 32.9 | 33.9 | 34.9 | 35.7 | | | | | | | 40.6 | 48.6 | |
| Male | DHA | 9743-0304 | Weight (g) | 1530 | 1880 | 2160 | 2375 | 2440 | | | | | 32.2 | 3628 | 5840 | |
| | | | Age (weeks pca) | 32.3 | 34.0 | 35.0 | 36.0 | 36.4 | | | | | | 38.1 | 50.6 | |
| Male | DHA + ARA | 9698-0305 | Weight (g) | 1120 | 1340 | 1550 | | | | | | | 20.9 | 2440 | 5525 | 6646 |
| | | | Age (weeks pca) | 30.7 | 32.6 | 33.6 | | | | | | | | 37.4 | 47.6 | 56.6 |
| Male | DHA + ARA | 9698-0308 | Weight (g) | 1410 | 1690 | 1870 | 2120 | | | | | | 32.0 | 3553 | 6007 | 7937 |
| | | | Age (weeks pca) | 31.1 | 32.4 | 33.3 | 34.3 | | | | | | | 40.3 | 47.6 | 57.3 |
| Male | DHA + ARA | 9699-0304 | Weight (g) | 1499 | 1689 | 1950 | 2355 | | | | | | 29.8 | 2355 | 3404 | 4993 |
| | | | Age (weeks pca) | 36.1 | 37.1 | 38.1 | 40.3 | | | | | | | 40.3 | 48.0 | 57.1 |
| Male | DHA + ARA | 9699-0305 | Weight (g) | 1056 | 1134 | 1290 | 1490 | | | | | | 17.2 | 2610 | 4256 | 5050 |
| | | | Age (weeks pca) | 32.0 | 33.0 | 34.0 | 35.7 | | | | | | | 40.6 | 48.7 | 57.6 |
| Male | DHA + ARA | 9700-0302 | Weight (g) | 1635 | 1880 | 2235 | 2570 | 2735 | | | | | 40.7 | 3255 | 5540 | 7380 |
| | | | Age (weeks pca) | 33.9 | 34.7 | 35.9 | 36.9 | 37.9 | | | | | | 39.7 | 47.7 | 56.7 |
| Male | DHA + ARA | 9701-0302 | Weight (g) | 1442 | 1686 | 2045 | 2835 | | | | | | 48.9 | 3240 | 5055 | 6600 |
| | | | Age (weeks pca) | 33.6 | 34.6 | 35.6 | 37.7 | | | | | | | 39.7 | 46.7 | 56.7 |
| Male | DHA + ARA | 9701-0306 | Weight (g) | 1587 | 2037 | 2245 | 2460 | 2756 | 3072 | 3228 | | | 41.4 | 3960 | 5200 | |
| | | | Age (weeks pca) | 32.3 | 33.4 | 34.4 | 35.3 | 36.3 | 37.3 | 37.7 | | | | 42.3 | 48.4 | |
| Male | DHA + ARA | 9701-0307 | Weight (g) | 1397 | 1710 | 1919 | 2932 | | | | | | 42.5 | 3445 | 5930 | 7475 |
| | | | Age (weeks pca) | 33.3 | 34.3 | 35.1 | 38.4 | | | | | | | 40.6 | 48.6 | 57.4 |
| Male | DHA + ARA | 9702-0301 | Weight (g) | 1670 | 1865 | 2160 | 2660 | | | | | | 36.0 | 3780 | 5250 | |
| | | | Age (weeks pca) | 32.0 | 33.0 | 34.0 | 36.0 | | | | | | | 40.6 | 47.6 | |
| Male | DHA + ARA | 9702-0303 | Weight (g) | 1650 | 1905 | 2660 | | | | | | | 40.7 | 3500 | 5160 | 6520 |
| | | | Age (weeks pca) | 32.9 | 33.9 | 36.4 | | | | | | | | 40.0 | 48.0 | 56.4 |
| Male | DHA + ARA | 9703-0301 | Weight (g) | 1255 | 1460 | 1745 | 2055 | 2415 | | | | | 42.3 | 4350 | 6020 | 6720 |
| | | | Age (weeks pca) | 29.4 | 30.4 | 31.3 | 32.3 | 33.4 | | | | | | 40.4 | 47.4 | 56.6 |
| Male | DHA + ARA | 9703-0305 | Weight (g) | 1440 | 1635 | 1830 | 2115 | 2390 | 2590 | | | | 34.1 | 3170 | 4330 | 5630 |
| | | | Age (weeks pca) | 32.0 | 33.0 | 34.0 | 35.0 | 36.1 | 36.9 | | | | | 40.0 | 47.9 | 56.7 |
| Male | DHA + ARA | 9704-0301 | Weight (g) | 1110 | 1270 | 1490 | 1740 | 2050 | | | | | 35.1 | 3220 | 5460 | 7050 |
| | | | Age (weeks pca) | 30.6 | 31.6 | 32.4 | 33.4 | 34.4 | | | | | | 39.9 | 47.7 | 56.7 |
| Male | DHA + ARA | 9704-0302 | Weight (g) | 1080 | 1230 | 1370 | 1520 | 1680 | 1840 | | | | 22.2 | 2570 | 6540 | 8050 |
| | | | Age (weeks pca) | 32.0 | 33.0 | 34.0 | 34.9 | 36.0 | 36.9 | | | | | 40.0 | 48.1 | 57.4 |
| Male | DHA + ARA | 9705-0301 | Weight (g) | 1300 | 1440 | 1620 | 1870 | | | | | | 27.0 | 2979 | 4400 | 5873 |
| | | | Age (weeks pca) | 32.7 | 33.7 | 34.7 | 35.7 | | | | | | | 40.1 | 48.1 | 58.0 |
| Male | DHA + ARA | 9705-0306 | Weight (g) | 1320 | 1490 | 1700 | 2020 | 2300 | | | | | 32.7 | 3631 | 5447 | 6809 |
| | | | Age (weeks pca) | 31.4 | 32.4 | 33.4 | 34.4 | 35.9 | | | | | | 39.9 | 47.9 | 56.9 |
| Male | DHA + ARA | 9705-0307 | Weight (g) | 1480 | 1650 | 1810 | 2240 | | | | | | 36.4 | 3007 | 5589 | 6596 |
| | | | Age (weeks pca) | 34.4 | 35.4 | 36.1 | 37.4 | | | | | | | 39.9 | 48.4 | 56.7 |
| Male | DHA + ARA | 9706-0305 | Weight (g) | 1330 | 1455 | 1660 | 1930 | | | | | | 31.4 | 2695 | 4820 | 6225 |
| | | | Age (weeks pca) | 33.9 | 34.4 | 35.4 | 36.6 | | | | | | | 39.9 | 48.1 | 58.1 |
| Male | DHA + ARA | 9706-0307 | Weight (g) | 1355 | 1585 | 1825 | 2270 | | | | | | 40.0 | 3585 | 5955 | 6925 |
| | | | Age (weeks pca) | 31.9 | 33.0 | 33.9 | 35.1 | | | | | | | 40.4 | 49.1 | 57.6 |
| Male | DHA + ARA | 9706-0309 | Weight (g) | 1620 | 1910 | 2150 | | | | | | | 40.3 | 3460 | 5255 | 5775 |
| | | | Age (weeks pca) | 34.1 | 35.3 | 36.0 | | | | | | | | 40.9 | 48.7 | 57.4 |
| Male | DHA + ARA | 9707-0301 | Weight (g) | 1553 | 1980 | 2280 | 2720 | | | | | | 41.5 | 3395 | 4950 | 6285 |
| | | | Age (weeks pca) | 32.6 | 34.3 | 35.3 | 36.6 | | | | | | | 40.1 | 47.9 | 56.9 |
| Male | DHA + ARA | 9707-0305 | Weight (g) | 1755 | 1990 | 2245 | 2505 | 2770 | | | | | 37.4 | | | |
| | | | Age (weeks pca) | 33.9 | 34.7 | 35.7 | 36.7 | 37.7 | | | | | | | | |
| Male | DHA + ARA | 9707-0310 | Weight (g) | 1620 | 1828 | 2140 | 3195 | | | | | | 44.8 | 3585 | 5170 | 6725 |
| | | | Age (weeks pca) | 32.7 | 33.7 | 34.7 | 37.9 | | | | | | | 39.7 | 47.9 | 56.3 |
| Male | DHA + ARA | 9708-0301 | Weight (g) | 1640 | 1880 | 2200 | 2420 | | | | | | 38.0 | 3730 | 4835 | 6185 |
| | | | Age (weeks pca) | 32.7 | 33.7 | 34.7 | 35.7 | | | | | | | 40.1 | 47.9 | 57.0 |
| Male | DHA + ARA | 9708-0304 | Weight (g) | 1680 | 2180 | | | | | | | | 55.6 | | | |
| | | | Age (weeks pca) | 34.6 | 35.9 | | | | | | | | | | | |
| Male | DHA + ARA | 9709-0303 | Weight (g) | 1470 | 1810 | | | | | | | | 48.6 | | | |
| | | | Age (weeks pca) | 32.6 | 33.6 | | | | | | | | | | | |
| Male | DHA + ARA | 9709-0305 | Weight (g) | 1410 | 1655 | 1900 | 2160 | | | | | | 35.6 | 2630 | 4570 | 5520 |
| | | | Age (weeks pca) | 34.4 | 35.4 | 36.4 | 37.4 | | | | | | | 39.7 | 47.7 | 57.1 |
| Male | DHA + ARA | 9712-0303 | Weight (g) | 1180 | 1210 | 1450 | 1590 | | | | | | 20.9 | 2520 | 3500 | 5010 |
| | | | Age (weeks pca) | 31.4 | 32.3 | 33.4 | 34.4 | | | | | | | 40.4 | 47.4 | 56.4 |
| Male | DHA + ARA | 9712-0305 | Weight (g) | 1325 | 1505 | 1785 | 2010 | 2300 | | | | | 34.1 | 3030 | 4350 | 5510 |
| | | | Age (weeks pca) | 31.5 | 32.5 | 33.5 | 34.5 | 35.6 | | | | | | 39.6 | 48.6 | 57.6 |
| Male | DHA + ARA | 9723-0301 | Weight (g) | 1630 | 1728 | 1961 | 2214 | | | | | | 28.4 | 3104 | | 5986 |
| | | | Age (weeks pca) | 33.9 | 34.9 | 35.9 | 36.9 | | | | | | | 40.3 | | 58.9 |
| Male | HM | 9698-0601 | | | | | | | | | | | | 3518 | 5497 | 6582 |
| | | | | | | | | | | | | | | 40.0 | 48.3 | 56.9 |
| Male | HM | 9698-0602 | | | | | | | | | | | | 3177 | 5220 | 6355 |
| | | | | | | | | | | | | | | 40.0 | 48.1 | 57.0 |
| Male | HM | 9698-0603 | | | | | | | | | | | | 3858 | 5447 | 6454 |
| | | | | | | | | | | | | | | 40.0 | 48.0 | 57.0 |
| Male | HM | 9698-0604 | | | | | | | | | | | | 4355 | 5092 | 6383 |
| | | | | | | | | | | | | | | 40.0 | 48.0 | 57.0 |
| Male | HM | 9698-0605 | | | | | | | | | | | | 3433 | 4979 | 6426 |
| | | | | | | | | | | | | | | 40.0 | 48.1 | 57.1 |
| Male | HM | 9699-0501 | | | | | | | | | | | | 3915 | 6639 | 7773 |
| | | | | | | | | | | | | | | 40.0 | 48.3 | 57.4 |
| Male | HM | 9699-0502 | | | | | | | | | | | | 3802 | 5787 | 7178 |
| | | | | | | | | | | | | | | 40.0 | 48.4 | 57.4 |
| Male | HM | 9701-0601 | | | | | | | | | | | | 3317 | 5555 | 7070 |
| | | | | | | | | | | | | | | 40.0 | 47.9 | 56.4 |
| Male | HM | 9701-0602 | | | | | | | | | | | | 3487 | 5833 | 8070 |
| | | | | | | | | | | | | | | 40.0 | 47.3 | 58.3 |
| Male | HM | 9701-0603 | | | | | | | | | | | | 3232 | 4940 | 5855 |
| | | | | | | | | | | | | | | 40.0 | 47.4 | 56.4 |
| Male | HM | 9701-0604 | | | | | | | | | | | | 3600 | 5215 | 6285 |
| | | | | | | | | | | | | | | 40.0 | 47.9 | 56.9 |
| Male | HM | 9701-0605 | | | | | | | | | | | | 3402 | 5575 | 7210 |
| | | | | | | | | | | | | | | 40.0 | 47.6 | 57.6 |
| Male | HM | 9701-0606 | | | | | | | | | | | | 3090 | 4485 | 5445 |
| | | | | | | | | | | | | | | 40.0 | 47.7 | 56.7 |
| Male | HM | 9702-0601 | | | | | | | | | | | | 3480 | 5780 | 6530 |
| | | | | | | | | | | | | | | 40.0 | 48.6 | 56.6 |
| Male | HM | 9702-0602 | | | | | | | | | | | | 3165 | 5060 | 6660 |
| | | | | | | | | | | | | | | 40.0 | 48.3 | 57.1 |
| Male | HM | 9703-0502 | | | | | | | | | | | | 2670 | 5420 | 7220 |
| | | | | | | | | | | | | | | 40.0 | 48.3 | 57.1 |
| Male | HM | 9703-0503 | | | | | | | | | | | | 4100 | 6740 | 8330 |
| | | | | | | | | | | | | | | 40.0 | 47.4 | 56.4 |
| Male | HM | 9703-0504 | | | | | | | | | | | | 3435 | 6000 | 7930 |
| | | | | | | | | | | | | | | 40.0 | 48.1 | 57.1 |
| Male | HM | 9704-0502 | | | | | | | | | | | | 3285 | 5220 | 6560 |
| | | | | | | | | | | | | | | 40.0 | 48.1 | 56.6 |
| Male | HM | 9704-0503 | | | | | | | | | | | | 3400 | 5200 | 6725 |
| | | | | | | | | | | | | | | 40.0 | 48.7 | 56.9 |
| Male | HM | 9705-0601 | | | | | | | | | | | | 3200 | 5617 | 6752 |
| | | | | | | | | | | | | | | 40.0 | 48.3 | 57.3 |
| Male | HM | 9705-0602 | | | | | | | | | | | | 3860 | 6227 | |
| | | | | | | | | | | | | | | 40.0 | 48.0 | |
| Male | HM | 9706-0601 | | | | | | | | | | | | 3152 | 5105 | 6545 |
| | | | | | | | | | | | | | | 40.0 | 49.0 | 57.0 |
| Male | HM | 9706-0602 | | | | | | | | | | | | 3557 | 5175 | 7315 |
| | | | | | | | | | | | | | | 40.0 | 47.4 | 57.7 |
| Male | HM | 9706-0603 | | | | | | | | | | | | 3192 | 5070 | 6970 |
| | | | | | | | | | | | | | | 40.0 | 47.9 | 56.7 |
| Male | HM | 9706-0604 | | | | | | | | | | | | 3461 | 4225 | 5525 |
| | | | | | | | | | | | | | | 40.0 | 48.0 | 57.1 |
| Male | HM | 9706-0605 | | | | | | | | | | | | 3870 | 6220 | 7660 |
| | | | | | | | | | | | | | | 40.0 | 48.1 | 56.4 |
| Male | HM | 9706-0606 | | | | | | | | | | | | 4315 | 5975 | 6720 |
| | | | | | | | | | | | | | | 40.0 | 48.3 | 56.6 |
| Male | HM | 9707-0601 | | | | | | | | | | | | 3263 | 4730 | 5825 |
| | | | | | | | | | | | | | | 40.0 | 48.1 | 57.0 |
| Male | HM | 9707-0602 | | | | | | | | | | | | 3206 | 4515 | 6220 |
| | | | | | | | | | | | | | | 40.0 | 48.1 | 57.7 |
| Male | HM | 9707-0603 | | | | | | | | | | | | 4256 | 6930 | 8810 |
| | | | | | | | | | | | | | | 40.0 | 48.0 | 57.0 |
| Male | HM | 9707-0604 | | | | | | | | | | | | 3419 | 5460 | 6130 |
| | | | | | | | | | | | | | | 40.0 | 48.0 | 56.7 |
| Male | HM | 9707-0605 | | | | | | | | | | | | 3433 | | |
| | | | | | | | | | | | | | | 40.0 | | |
| Male | HM | 9707-0606 | | | | | | | | | | | | 3603 | 5825 | |
| | | | | | | | | | | | | | | 40.0 | 48.4 | |
| Male | HM | 9707-0607 | | | | | | | | | | | | 3569 | 5410 | 6870 |
| | | | | | | | | | | | | | | 40.0 | 47.9 | 56.9 |
| Male | HM | 9707-0608 | | | | | | | | | | | | 3348 | 5135 | 6370 |
| | | | | | | | | | | | | | | 40.0 | 48.0 | 57.0 |
| Male | HM | 9707-0609 | | | | | | | | | | | | 3348 | | |
| | | | | | | | | | | | | | | 40.0 | | |
| Male | HM | 9708-0601 | | | | | | | | | | | | 3064 | 5220 | 6595 |
| | | | | | | | | | | | | | | 40.0 | 47.6 | 56.4 |
| Male | HM | 9708-0602 | | | | | | | | | | | | 4085 | | |
| | | | | | | | | | | | | | | 40.0 | | |
| Male | HM | 9708-0603 | | | | | | | | | | | | 3319 | 5135 | 6327 |
| | | | | | | | | | | | | | | 40.0 | 48.4 | 57.1 |
| Male | HM | 9708-0604 | | | | | | | | | | | | 3291 | | |
| | | | | | | | | | | | | | | 40.0 | | |
| Male | HM | 9708-0605 | | | | | | | | | | | | 3796 | | |
| | | | | | | | | | | | | | | 40.0 | | |
| Male | HM | 9708-0606 | | | | | | | | | | | | 4020 | 4645 | 5405 |
| | | | | | | | | | | | | | | 40.0 | 48.4 | 57.1 |
| Male | HM | 9708-0607 | | | | | | | | | | | | 3333 | 4043 | 5180 |
| | | | | | | | | | | | | | | 40.0 | 47.9 | 56.7 |
| Male | HM | 9709-0505 | | | | | | | | | | | | 3400 | | |
| | | | | | | | | | | | | | | 40.0 | | |
| Female | Control | 9698-0003* | Weight (g) | 1020 | 1050 | 1070 | 1080 | 1080 | 1060 | 1080 | 1070 | | 5.6 | | | |
| | | | Age (weeks pca) | 31.1 | 31.3 | 31.4 | 31.6 | 31.7 | 31.9 | 32.0 | 32.1 | | | | | |
| Female | Control | 9699-0001 | Weight (g) | 1464 | 1672 | 1862 | 2000 | 2145 | | | | | 24.1 | 2610 | 4369 | 5220 |
| | | | Age (weeks pca) | 32.7 | 33.7 | 34.7 | 35.7 | 36.7 | | | | | | 39.7 | 47.9 | 56.9 |
| Female | Control | 9699-0003 | Weight (g) | 1473 | 1629 | 1860 | 2497 | | | | | | 37.3 | 2780 | 4596 | 5816 |
| | | | Age (weeks pca) | 34.0 | 35.0 | 36.0 | 38.0 | | | | | | | 40.0 | 48.0 | 57.0 |
| Female | Control | 9701-0003 | Weight (g) | 1480 | 1633 | 1903 | 1975 | 2292 | | | | | 29.1 | 2675 | 4165 | 5200 |
| | | | Age (weeks pca) | 34.6 | 35.6 | 36.6 | 37.3 | 38.6 | | | | | | 40.6 | 48.6 | 55.6 |
| Female | Control | 9701-0005 | Weight (g) | 1174 | 1366 | 1555 | 1745 | 1976 | | | | | 28.3 | 3175 | 5140 | 6280 |
| | | | Age (weeks pca) | 30.7 | 31.7 | 32.7 | 33.7 | 34.7 | | | | | | 39.7 | 48.4 | 56.4 |
| Female | Control | 9701-0008 | Weight (g) | 1391 | 1569 | 1898 | 2198 | 2406 | | | | | 41.1 | 2980 | 4425 | 5815 |
| | | | Age (weeks pca) | 34.3 | 35.3 | 36.4 | 37.3 | 37.9 | | | | | | 40.4 | 47.4 | 56.4 |
| Female | Control | 9701-0011 | Weight (g) | 1050 | 1254 | 1492 | 1756 | 2044 | | | | | 36.6 | 2870 | 4420 | 5505 |
| | | | Age (weeks pca) | 30.6 | 31.4 | 32.4 | 33.4 | 34.4 | | | | | | 39.7 | 48.6 | 57.4 |
| Female | Control | 9702-0002 | Weight (g) | 1222 | 1371 | 1570 | 1750 | 1995 | 2390 | | | | 29.4 | 3380 | 4900 | |
| | | | Age (weeks pca) | 31.7 | 32.7 | 34.1 | 35.1 | 36.0 | 37.1 | | | | | 40.4 | 47.6 | |
| Female | Control | 9702-0004 | Weight (g) | 1454 | 1555 | 1840 | 2530 | | | | | | 31.6 | 3600 | 5160 | 6900 |
| | | | Age (weeks pca) | 31.0 | 31.9 | 33.1 | 36.0 | | | | | | | 39.9 | 47.7 | 56.7 |
| Female | Control | 9702-0010 | Weight (g) | 1775 | 2065 | 2410 | 2645 | | | | | | 42.2 | 3060 | 4820 | 6690 |
| | | | Age (weeks pca) | 34.0 | 35.0 | 36.0 | 37.0 | | | | | | | 39.9 | 48.3 | 57.6 |
| Female | Control | 9703-0002 | Weight (g) | 1170 | 1250 | 1390 | 1570 | 1825 | 2130 | | | | 26.4 | 3210 | 4750 | |
| | | | Age (weeks pca) | 29.1 | 30.4 | 31.3 | 32.4 | 33.4 | 34.3 | | | | | 39.6 | 47.4 | |
| Female | Control | 9703-0005 | Weight (g) | 1420 | 1590 | 1765 | 1900 | 2220 | | | | | 29.5 | 2610 | 4330 | 5640 |
| | | | Age (weeks pca) | 31.4 | 32.3 | 33.3 | 33.9 | 35.3 | | | | | | 37.3 | 46.0 | 55.0 |
| Female | Control | 9703-0008 | Weight (g) | 1495 | 1715 | 2095 | 2445 | 2685 | | | | | 48.3 | 3360 | 4780 | 6410 |
| | | | Age (weeks pca) | 33.0 | 34.0 | 35.0 | 36.0 | 36.6 | | | | | | 40.1 | 47.7 | 56.1 |
| Female | Control | 9705-0004 | Weight (g) | 1120 | 1290 | 1490 | 1660 | | | | | | 28.3 | 2722 | 4085 | 5646 |
| | | | Age (weeks pca) | 31.3 | 32.3 | 33.3 | 34.0 | | | | | | | 39.7 | 46.6 | 55.0 |
| Female | Control | 9706-0003 | Weight (g) | 1515 | 1673 | 1965 | 2330 | | | | | | 37.9 | | | |
| | | | Age (weeks pca) | 35.1 | 36.3 | 37.1 | 38.3 | | | | | | | | | |
| Female | Control | 9706-0005 | Weight (g) | 1485 | 1610 | 1805 | 2150 | | | | | | 31.7 | 2740 | 4165 | 5305 |
| | | | Age (weeks pca) | 33.0 | 33.7 | 34.7 | 36.0 | | | | | | | 40.0 | 48.1 | 57.3 |
| Female | Control | 9706-0009 | Weight (g) | 1525 | 1620 | 1960 | | | | | | | 31.6 | 3640 | 5495 | 7225 |
| | | | Age (weeks pca) | 32.3 | 32.9 | 34.3 | | | | | | | | 40.3 | 47.6 | 53.4 |
| Female | Control | 9706-0010 | Weight (g) | 1905 | 2185 | | | | | | | | 56.0 | 3655 | 5390 | 6535 |
| | | | Age (weeks pca) | 34.3 | 35.0 | | | | | | | | | 40.0 | 48.4 | 56.7 |
| Female | Control | 9706-0013 | Weight (g) | 1185 | 1270 | 1585 | 1810 | | | | | | 31.1 | 2680 | 3800 | |
| | | | Age (weeks pca) | 31.6 | 32.4 | 33.6 | 34.6 | | | | | | | 40.1 | 48.4 | |
| Female | Control | 9706-0016 | Weight (g) | 1510 | 1765 | 1935 | | | | | | | 32.6 | 3320 | 4535 | 5297 |
| | | | Age (weeks pca) | 32.0 | 33.1 | 33.9 | | | | | | | | 40.7 | 48.7 | 56.6 |
| Female | Control | 9707-0003 | Weight (g) | 1465 | 1505 | 1655 | 2010 | 2325 | 2765 | | | | 30.2 | 3110 | 4125 | 4995 |
| | | | Age (weeks pca) | 32.0 | 32.6 | 33.6 | 35.3 | 36.4 | 38.3 | | | | | 40.1 | 48.1 | 57.1 |
| Female | Control | 9707-0006 | Weight (g) | 1866 | 3430 | 3430 | | | | | | | 41.2 | 3430 | 5385 | 7250 |
| | | | Age (weeks pca) | 34.6 | 40.0 | 40.0 | | | | | | | | 40.0 | 48.9 | 57.3 |
| Female | Control | 9707-1006 | Weight (g) | 1815 | 3330 | 3330 | | | | | | | 39.9 | 3330 | 5490 | 6920 |
| | | | Age (weeks pca) | 34.6 | 40.0 | 40.0 | | | | | | | | 40.0 | 48.9 | 57.3 |
| Female | Control | 9708-0001 | Weight (g) | 1410 | 1600 | 1850 | 2050 | | | | | | 27.2 | 2910 | 4734 | |
| | | | Age (weeks pca) | 33.4 | 34.4 | 35.4 | 36.9 | | | | | | | 40.6 | 48.4 | |
| Female | Control | 9708-0003 | Weight (g) | 940.0 | 970.0 | | | | | | | | 4.3 | | | |
| | | | Age (weeks pca) | 30.0 | 31.0 | | | | | | | | | | | |
| Female | Control | 9708-0008 | Weight (g) | 1380 | 1605 | 1860 | 2180 | | | | | | 33.1 | 2582 | 4110 | 5361 |
| | | | Age (weeks pca) | 32.9 | 33.7 | 34.9 | 36.3 | | | | | | | 39.3 | 47.4 | 57.1 |
| Female | Control | 9709-0002 | Weight (g) | 1980 | 2225 | 2400 | | | | | | | 30.0 | | | |
| | | | Age (weeks pca) | 32.7 | 33.7 | 34.7 | | | | | | | | | | |
| Female | Control | 9709-0005 | Weight (g) | 1175 | 1425 | 1665 | 1945 | 2200 | | | | | 32.3 | 2975 | 4700 | 5900 |
| | | | Age (weeks pca) | 31.9 | 33.3 | 34.6 | 35.6 | 36.3 | | | | | | 39.6 | 48.4 | 56.7 |
| Female | Control | 9712-0005 | Weight (g) | 972.0 | 1145 | 1290 | 1490 | 1695 | | | | | 25.6 | 2930 | 4450 | 5880 |
| | | | Age (weeks pca) | 29.1 | 30.1 | 31.1 | 32.1 | 33.1 | | | | | | 40.3 | 47.6 | 57.1 |
| Female | Control | 9712-0006 | Weight (g) | 1203 | 1358 | 1585 | 1790 | | | | | | 28.4 | 3030 | 4560 | 6230 |
| | | | Age (weeks pca) | 31.9 | 32.9 | 33.9 | 34.9 | | | | | | | 39.7 | 48.0 | 57.0 |
| Female | Control | 9743-0003 | Weight (g) | 1300 | 1520 | 1740 | 1890 | | | | | | 24.0 | | 4000 | 5160 |
| | | | Age (weeks pca) | 31.6 | 33.4 | 34.1 | 35.1 | | | | | | | | 48.4 | 57.4 |
| Female | Control | 9746-0001 | Weight (g) | 1420 | 1740 | 2075 | 2320 | 2625 | | | | | 42.7 | 3170 | 4145 | 5192 |
| | | | Age (weeks pca) | 32.6 | 33.6 | 34.6 | 35.6 | 36.6 | | | | | | 39.7 | 47.6 | 56.6 |
| Female | DHA | 9698-0004 | Weight (g) | 1410 | 1650 | 1890 | 2140 | | | | | | 34.7 | 3787 | 4795 | 6291 |
| | | | Age (weeks pca) | 30.1 | 31.1 | 32.1 | 33.1 | | | | | | | 40.0 | 48.0 | 57.0 |
| Female | DHA | 9698-0006 | Weight (g) | 1110 | 1240 | 1420 | 1720 | | | | | | 28.7 | | | |
| | | | Age (weeks pca) | 30.7 | 31.7 | 32.7 | 33.7 | | | | | | | | | |
| Female | DHA | 9698-0009 | Weight (g) | 1205 | 1310 | 1520 | 1630 | 2020 | | | | | 25.9 | 2891 | 3979 | 5121 |
| | | | Age (weeks pca) | 30.3 | 31.4 | 32.4 | 33.1 | 34.9 | | | | | | 40.0 | 48.0 | 57.0 |
| Female | DHA | 9698-0307 | Weight (g) | 1790 | 2110 | 2450 | | | | | | | 29.7 | 3135 | 5185 | 6695 |
| | | | Age (weeks pca) | 34.4 | 35.7 | 37.6 | | | | | | | | 39.4 | 47.4 | 56.4 |
| Female | DHA | 9699-0002 | Weight (g) | 1313 | 1477 | 1669 | 1929 | 2380 | | | | | 36.9 | 3177 | 5787 | 7093 |
| | | | Age (weeks pca) | 32.9 | 33.9 | 34.9 | 35.9 | 36.9 | | | | | | 39.7 | 47.7 | 56.7 |
| Female | DHA | 9700-0001 | Weight (g) | 1580 | 1820 | 2050 | 2295 | 2500 | | | | | 34.5 | 3210 | 5110 | 6300 |
| | | | Age (weeks pca) | 32.4 | 33.4 | 34.3 | 35.3 | 36.3 | | | | | | 40.1 | 48.1 | 57.1 |
| Female | DHA | 9701-0001 | Weight (g) | 1300 | 1356 | 1586 | 1924 | 2125 | | | | | 34.2 | 2910 | 4325 | 5625 |
| | | | Age (weeks pca) | 33.0 | 34.0 | 35.0 | 36.0 | 36.6 | | | | | | 39.6 | 48.0 | 57.0 |
| Female | DHA | 9701-0004 | Weight (g) | 1108 | 1261 | 1441 | 1671 | 1897 | | | | | 28.4 | 3020 | 4855 | 6040 |
| | | | Age (weeks pca) | 30.7 | 31.7 | 32.7 | 33.7 | 34.7 | | | | | | 39.7 | 48.4 | 56.4 |
| Female | DHA | 9701-0012 | Weight (g) | 1674 | 1928 | 2151 | 2311 | 2685 | 2685 | | | | 30.1 | 2685 | | |
| | | | Age (weeks pca) | 34.9 | 35.9 | 36.9 | 37.6 | 39.6 | 39.6 | | | | | 39.6 | | |
| Female | DHA | 9701-0014 | Weight (g) | 1422 | 1631 | 1858 | 2455 | | | | | | 37.2 | 2970 | 4605 | 5140 |
| | | | Age (weeks pca) | 33.9 | 34.9 | 35.9 | 37.9 | | | | | | | 39.9 | 47.7 | 56.9 |
| Female | DHA | 9702-0001 | Weight (g) | 1780 | 2115 | 2390 | 3000 | | | | | | 35.8 | 3850 | 5610 | 6600 |
| | | | Age (weeks pca) | 31.6 | 32.9 | 33.9 | 36.4 | | | | | | | 40.0 | 49.6 | 57.0 |
| Female | DHA | 9702-0006 | Weight (g) | 1850 | 2005 | 2650 | 2650 | | | | | | 27.3 | 2650 | 4450 | 6020 |
| | | | Age (weeks pca) | 35.4 | 36.1 | 39.6 | 39.6 | | | | | | | 39.6 | 48.4 | 56.4 |
| Female | DHA | 9702-0007 | Weight (g) | 1285 | 1459 | 1780 | 1965 | 2035 | | | | | 29.6 | | | |
| | | | Age (weeks pca) | 31.1 | 32.1 | 33.6 | 34.4 | 34.9 | | | | | | | | |
| Female | DHA | 9702-0008 | Weight (g) | 1605 | 1930 | 3540 | 3540 | | | | | | 51.3 | 3540 | 5920 | 7820 |
| | | | Age (weeks pca) | 34.1 | 35.1 | 39.6 | 39.6 | | | | | | | 39.6 | 47.6 | 57.1 |
| Female | DHA | 9703-0003 | Weight (g) | 1255 | 1355 | 1535 | 1845 | 2150 | | | | | 34.8 | 2430 | 4130 | 5010 |
| | | | Age (weeks pca) | 34.4 | 35.1 | 36.1 | 37.1 | 38.1 | | | | | | 39.4 | 48.0 | 56.1 |
| Female | DHA | 9703-0004 | Weight (g) | 1170 | 1340 | 1550 | 1795 | 2225 | | | | | 33.9 | 2870 | 4610 | 6490 |
| | | | Age (weeks pca) | 32.6 | 33.3 | 34.3 | 35.3 | 37.0 | | | | | | 39.4 | 48.1 | 57.1 |
| Female | DHA | 9703-0009 | Weight (g) | 1570 | 1830 | 2095 | 2395 | 2655 | | | | | 34.6 | 3160 | 4480 | 5570 |
| | | | Age (weeks pca) | 33.3 | 34.3 | 35.1 | 36.3 | 37.9 | | | | | | 40.4 | 48.4 | 58.0 |
| Female | DHA | 9704-0004 | Weight (g) | 1440 | 1670 | 1740 | | | | | | | 30.5 | 3100 | 5830 | 8630 |
| | | | Age (weeks pca) | 33.6 | 34.6 | 35.0 | | | | | | | | 40.0 | 48.0 | 57.0 |
| Female | DHA | 9704-0005 | Weight (g) | 1050 | 1310 | 1490 | 1700 | 1890 | | | | | 30.0 | 3360 | 4860 | 6100 |
| | | | Age (weeks pca) | 29.7 | 30.9 | 31.7 | 32.7 | 33.7 | | | | | | 39.6 | 48.0 | 57.0 |
| Female | DHA | 9705-0001 | Weight (g) | 1220 | 1370 | 1590 | 1880 | 2098 | | | | | 31.9 | 3092 | 4795 | 5986 |
| | | | Age (weeks pca) | 32.7 | 33.6 | 34.7 | 35.7 | 36.7 | | | | | | 40.1 | 48.1 | 57.1 |
| Female | DHA | 9706-0006 | Weight (g) | 1270 | 1405 | 1630 | 1930 | | | | | | 31.7 | 2705 | 4145 | 5320 |
| | | | Age (weeks pca) | 33.0 | 33.7 | 34.7 | 36.0 | | | | | | | 40.0 | 48.1 | 57.3 |
| Female | DHA | 9706-0008 | Weight (g) | 990.0 | 1188 | 1345 | 1485 | | | | | | 23.0 | 2120 | | |
| | | | Age (weeks pca) | 33.4 | 34.6 | 35.7 | 36.4 | | | | | | | 39.9 | | |
| Female | DHA | 9706-0012 | Weight (g) | 1610 | 1830 | 2130 | 2280 | | | | | | 32.5 | 3530 | 4790 | |
| | | | Age (weeks pca) | 31.6 | 32.4 | 33.6 | 34.6 | | | | | | | 40.1 | 48.4 | |
| Female | DHA | 9706-0014 | Weight (g) | 1080 | 1170 | 1395 | 1560 | 1804 | | | | | 26.2 | 3295 | 5600 | 7675 |
| | | | Age (weeks pca) | 31.3 | 32.6 | 33.4 | 34.4 | 35.3 | | | | | | 40.6 | 49.4 | 58.0 |
| Female | DHA | 9707-0004 | Weight (g) | 1635 | 1771 | 2850 | | | | | | | 38.1 | 3045 | 4595 | 5765 |
| | | | Age (weeks pca) | 34.0 | 35.0 | 38.7 | | | | | | | | 40.0 | 48.0 | 57.0 |
| Female | DHA | 9707-0308 | Weight (g) | 2005 | 3440 | 3440 | | | | | | | 42.2 | 3440 | 4800 | 6360 |
| | | | Age (weeks pca) | 34.4 | 39.3 | 39.3 | | | | | | | | 39.3 | 47.3 | 57.7 |
| Female | DHA | 9708-0004 | Weight (g) | 1460 | 1665 | 1955 | 2280 | 2485 | | | | | 38.1 | | | |
| | | | Age (weeks pca) | 32.6 | 33.6 | 34.6 | 35.6 | 36.6 | | | | | | | | |
| Female | DHA | 9708-0006 | Weight (g) | 1485 | 1775 | 2110 | 2380 | | | | | | 39.5 | 3010 | 4620 | 6530 |
| | | | Age (weeks pca) | 33.7 | 34.7 | 35.7 | 37.0 | | | | | | | 40.1 | 48.1 | 57.0 |
| Female | DHA | 9709-0001 | Weight (g) | 1250 | 1490 | 1755 | 1970 | 2250 | 2520 | | | | 33.8 | 3500 | | |
| | | | Age (weeks pca) | 29.6 | 31.0 | 32.0 | 33.0 | 34.0 | 35.0 | | | | | 40.1 | | |
| Female | DHA | 9709-0003 | Weight (g) | 1540 | 1725 | 2015 | 2155 | | | | | | 30.5 | 2580 | 4080 | 5420 |
| | | | Age (weeks pca) | 34.4 | 35.4 | 36.4 | 37.4 | | | | | | | 40.3 | 47.7 | 57.1 |
| Female | DHA | 9712-0001 | Weight (g) | 987.0 | 1120 | 1270 | 1470 | 1685 | | | | | 24.9 | 2940 | 3980 | 5250 |
| | | | Age (weeks pca) | 30.0 | 31.0 | 32.0 | 33.0 | 34.0 | | | | | | 40.1 | 48.1 | 57.1 |
| Female | DHA | 9712-0002 | Weight (g) | 1060 | 1230 | 1430 | | | | | | | 26.4 | | | |
| | | | Age (weeks pca) | 32.7 | 33.7 | 34.7 | | | | | | | | | | |
| Female | DHA | 9712-0007 | Weight (g) | 1082 | 1230 | 1440 | 1650 | | | | | | 27.3 | 2425 | 4250 | 5340 |
| | | | Age (weeks pca) | 32.7 | 33.7 | 34.7 | 35.7 | | | | | | | 39.7 | 47.9 | 56.9 |
| Female | DHA | 9743-0001 | Weight (g) | 1000 | 1170 | 1470 | 1800 | 1930 | | | | | 33.5 | | 4140 | 5400 |
| | | | Age (weeks pca) | 32.1 | 33.1 | 34.4 | 35.7 | 36.1 | | | | | | | 48.3 | 57.3 |
| Female | DHA | 9743-0002 | Weight (g) | 1380 | 1570 | 1845 | 1975 | | | | | | 29.7 | | 4540 | 5160 |
| | | | Age (weeks pca) | 32.1 | 33.3 | 34.1 | 35.1 | | | | | | | | 48.4 | 57.4 |
| Female | DHA + ARA | 9698-0001 | Weight (g) | 1550 | 1690 | 2000 | 2380 | | | | | | 37.1 | 3530 | 5348 | 6582 |
| | | | Age (weeks pca) | 31.6 | 32.6 | 33.6 | 34.9 | | | | | | | 40.0 | 47.7 | 56.7 |
| Female | DHA + ARA | 9698-0002 | Weight (g) | 1580 | 1870 | 2130 | 2260 | | | | | | 31.8 | 3241 | | |
| | | | Age (weeks pca) | 32.6 | 33.7 | 34.6 | 35.7 | | | | | | | 40.7 | | |
| Female | DHA + ARA | 9699-0004 | Weight (g) | 985.0 | 1122 | 1283 | 1536 | 1788 | | | | | 28.9 | 3177 | 5107 | 6979 |
| | | | Age (weeks pca) | 31.0 | 32.0 | 33.0 | 34.0 | 35.0 | | | | | | 41.3 | 48.3 | 57.3 |
| Female | DHA + ARA | 9699-0005 | Weight (g) | 1330 | 1542 | 1688 | 2000 | 2330 | | | | | 35.1 | 4029 | 6752 | 8341 |
| | | | Age (weeks pca) | 31.9 | 32.9 | 33.9 | 34.9 | 35.9 | | | | | | 40.0 | 48.0 | 57.0 |
| Female | DHA + ARA | 9700-0002 | Weight (g) | 1315 | 1525 | 1885 | 2035 | 2220 | 2480 | | | | 31.9 | 3340 | 4930 | 6420 |
| | | | Age (weeks pca) | 30.3 | 31.3 | 32.3 | 33.3 | 34.1 | 35.6 | | | | | 40.3 | 48.1 | 57.1 |
| Female | DHA + ARA | 9701-0002 | Weight (g) | 1398 | 1609 | 1887 | 2210 | 2420 | | | | | 37.8 | 2930 | 5115 | 6525 |
| | | | Age (weeks pca) | 33.4 | 34.4 | 35.4 | 36.4 | 37.4 | | | | | | 39.4 | 48.4 | 56.4 |
| Female | DHA + ARA | 9701-0006 | Weight (g) | 1720 | 1859 | 2113 | 2456 | 2728 | | | | | 38.3 | 3600 | 5045 | 6270 |
| | | | Age (weeks pca) | 32.3 | 33.3 | 34.3 | 35.3 | 36.1 | | | | | | 40.3 | 48.0 | 57.3 |
| Female | DHA + ARA | 9701-0007 | Weight (g) | 1469 | 1427 | 1590 | 1982 | 2227 | | | | | 29.8 | 2680 | 4935 | 6955 |
| | | | Age (weeks pca) | 33.7 | 34.9 | 35.7 | 36.7 | 37.7 | | | | | | 39.9 | 47.9 | 56.9 |
| Female | DHA + ARA | 9701-0010 | Weight (g) | 1488 | 1703 | 1978 | 2234 | 2433 | 2759 | | | | 34.6 | 3500 | 5545 | |
| | | | Age (weeks pca) | 32.3 | 33.4 | 34.4 | 35.3 | 36.1 | 37.7 | | | | | 41.1 | 48.4 | |
| Female | DHA + ARA | 9701-0013 | Weight (g) | 1841 | 2019 | | | | | | | | 35.6 | | 4545 | 5550 |
| | | | Age (weeks pca) | 33.0 | 33.7 | | | | | | | | | | 48.7 | 57.4 |
| Female | DHA + ARA | 9702-0003 | Weight (g) | 1293 | 1488 | 1820 | 2155 | 2400 | | | | | 39.9 | 4190 | 6220 | 7500 |
| | | | Age (weeks pca) | 30.1 | 31.1 | 32.1 | 33.4 | 34.1 | | | | | | 40.0 | 48.4 | 56.9 |
| Female | DHA + ARA | 9702-0005 | Weight (g) | 1895 | 2060 | 2300 | 2525 | 2710 | | | | | 29.9 | 3025 | 4300 | 5340 |
| | | | Age (weeks pca) | 34.0 | 35.0 | 36.0 | 37.0 | 38.0 | | | | | | 40.0 | 47.4 | 56.4 |
| Female | DHA + ARA | 9702-0009 | Weight (g) | 1725 | 2000 | 2230 | 2595 | 2655 | | | | | 40.9 | 2905 | 4680 | 6410 |
| | | | Age (weeks pca) | 34.0 | 35.0 | 36.0 | 37.0 | 37.3 | | | | | | 39.9 | 48.3 | 57.6 |
| Female | DHA + ARA | 9703-0001 | Weight (g) | 1145 | 1255 | 1450 | 1680 | 1955 | | | | | 28.9 | 3030 | 4250 | 5420 |
| | | | Age (weeks pca) | 31.3 | 32.1 | 33.1 | 34.3 | 35.3 | | | | | | 41.0 | 48.1 | 57.3 |
| Female | DHA + ARA | 9703-0006 | Weight (g) | 1865 | 2200 | 2560 | 2880 | | | | | | 49.1 | 3600 | 5400 | 6650 |
| | | | Age (weeks pca) | 34.0 | 35.0 | 35.9 | 37.0 | | | | | | | 40.0 | 48.1 | 56.7 |
| Female | DHA + ARA | 9703-0007 | Weight (g) | 1390 | 1495 | 1620 | 1880 | 2030 | 2240 | | | | 27.4 | 2850 | 4190 | 5850 |
| | | | Age (weeks pca) | 32.0 | 33.1 | 34.0 | 35.0 | 35.7 | 36.6 | | | | | 40.0 | 47.9 | 56.7 |
| Female | DHA + ARA | 9704-0002 | Weight (g) | 960.0 | 1090 | 1200 | 1370 | 1570 | 1780 | 2070 | | | 26.7 | 3110 | 5150 | 6800 |
| | | | Age (weeks pca) | 29.0 | 30.0 | 30.9 | 31.9 | 32.9 | 33.9 | 34.9 | | | | 40.0 | 48.0 | 57.3 |
| Female | DHA + ARA | 9704-0003 | Weight (g) | 1690 | 1840 | | | | | | | | 30.0 | 4000 | 5400 | 6640 |
| | | | Age (weeks pca) | 32.7 | 33.4 | | | | | | | | | 40.0 | 48.0 | 57.0 |
| Female | DHA + ARA | 9705-0003 | Weight (g) | 1760 | 2260 | 2500 | 2920 | | | | | | 49.8 | 3376 | 5107 | 6894 |
| | | | Age (weeks pca) | 34.4 | 35.7 | 36.6 | 37.7 | | | | | | | 39.9 | 48.4 | 56.9 |
| Female | DHA + ARA | 9705-0005* | Weight (g) | 1075 | 1120 | 1185 | 1280 | 1310 | 1310 | 1265 | 1350 | 1380 | 22.1 | 2600 | 4000 | 5050 |
| | | | Age (weeks pca) | 31.1 | 31.4 | 31.7 | 32.1 | 32.4 | 32.7 | 33.0 | 33.3 | 33.4 | | 40.4 | 48.0 | 57.0 |
| Female | DHA + ARA | 9706-0001 | Weight (g) | 1290 | 1515 | 1685 | 2060 | | | | | | 34.5 | 4100 | 6550 | 7655 |
| | | | Age (weeks pca) | 31.7 | 32.9 | 33.7 | 34.9 | | | | | | | 40.1 | 48.6 | 56.7 |
| Female | DHA + ARA | 9706-0002 | Weight (g) | 1395 | 1710 | 1884 | 2275 | | | | | | 34.8 | 2845 | 4645 | 5550 |
| | | | Age (weeks pca) | 31.9 | 33.0 | 33.9 | 35.4 | | | | | | | 40.3 | 48.9 | 57.3 |
| Female | DHA + ARA | 9706-0004 | Weight (g) | 1550 | 1705 | 2050 | | | | | | | 36.1 | 2645 | 4225 | 4935 |
| | | | Age (weeks pca) | 36.7 | 37.6 | 38.7 | | | | | | | | 41.7 | 49.7 | 58.0 |
| Female | DHA + ARA | 9706-0007 | Weight (g) | 1235 | 1490 | 1820 | 1930 | | | | | | 34.3 | 2505 | | |
| | | | Age (weeks pca) | 33.4 | 34.6 | 35.7 | 36.4 | | | | | | | 40.3 | | |
| Female | DHA + ARA | 9706-0011 | Weight (g) | 1900 | 2105 | | | | | | | | 41.0 | 3430 | 5175 | 6140 |
| | | | Age (weeks pca) | 34.3 | 35.0 | | | | | | | | | 40.0 | 48.4 | 56.7 |
| Female | DHA + ARA | 9706-0015 | Weight (g) | 1670 | 1975 | 2210 | | | | | | | 41.6 | 3005 | 4465 | 5810 |
| | | | Age (weeks pca) | 34.6 | 35.6 | 36.4 | | | | | | | | 40.9 | 48.4 | 57.6 |
| Female | DHA + ARA | 9706-0017 | Weight (g) | 1465 | 1700 | 1895 | 2170 | | | | | | 33.4 | | | |
| | | | Age (weeks pca) | 32.3 | 33.4 | 34.3 | 35.3 | | | | | | | | | |
| Female | DHA + ARA | 9707-0002 | Weight (g) | 1775 | 2240 | 2385 | 2610 | | | | | | 33.2 | | | |
| | | | Age (weeks pca) | 34.3 | 36.0 | 36.9 | 37.9 | | | | | | | | | |
| Female | DHA + ARA | 9708-0002 | Weight (g) | 1535 | 1700 | 1980 | 2200 | | | | | | 32.5 | 2724 | 4645 | 6315 |
| | | | Age (weeks pca) | 33.0 | 34.0 | 35.0 | 36.0 | | | | | | | 38.1 | 47.6 | 55.4 |
| Female | DHA + ARA | 9708-0005 | Weight (g) | 1125 | 1345 | 1610 | 1980 | | | | | | 40.4 | 3121 | 5855 | 7875 |
| | | | Age (weeks pca) | 32.4 | 33.4 | 34.4 | 35.4 | | | | | | | 39.4 | 47.4 | 57.4 |
| Female | DHA + ARA | 9708-0007 | Weight (g) | 1200 | 1440 | 1680 | 1975 | | | | | | 36.6 | | | |
| | | | Age (weeks pca) | 31.3 | 32.3 | 33.3 | 34.3 | | | | | | | | | |
| Female | DHA + ARA | 9709-0004 | Weight (g) | 1350 | 1560 | 1885 | 2250 | 2475 | | | | | 37.0 | 3295 | 5250 | 6685 |
| | | | Age (weeks pca) | 31.9 | 33.3 | 34.6 | 35.6 | 36.3 | | | | | | 39.7 | 48.4 | 56.7 |
| Female | DHA + ARA | 9712-0003 | Weight (g) | 1283 | 1410 | 1590 | 1850 | 2010 | | | | | 27.1 | 2580 | 4130 | 5640 |
| | | | Age (weeks pca) | 32.0 | 33.0 | 34.0 | 35.0 | 36.0 | | | | | | 40.0 | 48.2 | 57.5 |
| Female | DHA + ARA | 9712-0004 | Weight (g) | 1575 | 1780 | 1890 | 2080 | 2530 | | | | | 29.7 | 3220 | 4920 | 6600 |
| | | | Age (weeks pca) | 33.0 | 34.0 | 34.6 | 35.6 | 37.6 | | | | | | 40.3 | 48.1 | 57.1 |
| Female | DHA + ARA | 9712-0008 | Weight (g) | 1590 | 1780 | 1990 | 2475 | | | | | | 37.2 | 2960 | 4470 | 5760 |
| | | | Age (weeks pca) | 34.0 | 35.0 | 35.8 | 37.4 | | | | | | | 40.1 | 48.1 | 57.1 |
| Female | DHA + ARA | 9746-0002 | Weight (g) | 1249 | 1429 | 1597 | 1814 | 2110 | | | | | 30.1 | 2680 | 4010 | 5362 |
| | | | Age (weeks pca) | 32.7 | 33.7 | 34.7 | 35.7 | 36.7 | | | | | | 39.9 | 46.9 | 56.9 |
| Female | HM | 9698-0501 | | | | | | | | | | | | 3546 | 4880 | |
| | | | | | | | | | | | | | | 40.0 | 48.3 | |
| Female | HM | 9698-0502 | | | | | | | | | | | | 3518 | 5972 | |
| | | | | | | | | | | | | | | 40.0 | 47.9 | |
| Female | HM | 9698-0503 | | | | | | | | | | | | 3390 | 4213 | 5319 |
| | | | | | | | | | | | | | | 40.0 | 48.3 | 57.1 |
| Female | HM | 9698-0504 | | | | | | | | | | | | 3383 | 5234 | 6667 |
| | | | | | | | | | | | | | | 40.0 | 48.7 | 57.9 |
| Female | HM | 9698-0505 | | | | | | | | | | | | 3646 | 4638 | 5653 |
| | | | | | | | | | | | | | | 40.0 | 48.3 | 57.0 |
| Female | HM | 9699-0601 | | | | | | | | | | | | 2582 | 4766 | 5731 |
| | | | | | | | | | | | | | | 40.0 | 49.0 | 57.0 |
| Female | HM | 9699-0602 | | | | | | | | | | | | 4284 | 4823 | 5986 |
| | | | | | | | | | | | | | | 40.0 | 48.0 | 57.0 |
| Female | HM | 9699-0603 | | | | | | | | | | | | 3716 | 4482 | 5674 |
| | | | | | | | | | | | | | | 40.0 | 47.7 | 56.7 |
| Female | HM | 9699-0604 | | | | | | | | | | | | 3660 | 4738 | 6355 |
| | | | | | | | | | | | | | | 40.0 | 48.0 | 57.0 |
| Female | HM | 9699-0605 | | | | | | | | | | | | 3433 | 5617 | 7603 |
| | | | | | | | | | | | | | | 40.0 | 48.4 | 57.6 |
| Female | HM | 9701-0501 | | | | | | | | | | | | 3884 | 5630 | 6450 |
| | | | | | | | | | | | | | | 40.0 | 47.7 | 57.7 |
| Female | HM | 9701-0502 | | | | | | | | | | | | 3858 | 5420 | 6700 |
| | | | | | | | | | | | | | | 40.0 | 48.6 | 57.6 |
| Female | HM | 9701-0503 | | | | | | | | | | | | 3430 | 4265 | 5085 |
| | | | | | | | | | | | | | | 40.0 | 47.4 | 57.4 |
| Female | HM | 9701-0504 | | | | | | | | | | | | 3317 | 5020 | 6230 |
| | | | | | | | | | | | | | | 40.0 | 48.1 | 57.1 |
| Female | HM | 9702-0501 | | | | | | | | | | | | 3302 | 5540 | 6630 |
| | | | | | | | | | | | | | | 40.0 | 47.7 | 56.7 |
| Female | HM | 9702-0502 | | | | | | | | | | | | 2658 | 5310 | 6800 |
| | | | | | | | | | | | | | | 40.0 | 47.4 | 57.1 |
| Female | HM | 9702-0503 | | | | | | | | | | | | 2895 | 3430 | 4530 |
| | | | | | | | | | | | | | | 40.0 | 47.7 | 57.4 |
| Female | HM | 9702-0504 | | | | | | | | | | | | 3401 | 5390 | 6270 |
| | | | | | | | | | | | | | | 40.0 | 48.0 | 57.4 |
| Female | HM | 9702-0505 | | | | | | | | | | | | 3141 | 4210 | 5320 |
| | | | | | | | | | | | | | | 40.0 | 47.9 | 57.0 |
| Female | HM | 9702-0506 | | | | | | | | | | | | 3762 | 6040 | 7600 |
| | | | | | | | | | | | | | | 40.0 | 48.9 | 57.7 |
| Female | HM | 9702-0507 | | | | | | | | | | | | 2718 | 4050 | 4940 |
| | | | | | | | | | | | | | | 40.0 | 48.9 | 57.4 |
| Female | HM | 9702-0508 | | | | | | | | | | | | 2927 | 4240 | 5860 |
| | | | | | | | | | | | | | | 40.0 | 47.4 | 57.0 |
| Female | HM | 9703-0501 | | | | | | | | | | | | 4085 | 5260 | 6360 |
| | | | | | | | | | | | | | | 40.0 | 48.1 | 57.1 |
| Female | HM | 9703-0505 | | | | | | | | | | | | 3390 | 5760 | 7670 |
| | | | | | | | | | | | | | | 40.0 | 48.3 | 57.3 |
| Female | HM | 9703-0506 | | | | | | | | | | | | 3405 | 6170 | 7490 |
| | | | | | | | | | | | | | | 40.0 | 47.9 | 56.9 |
| Female | HM | 9703-0507 | | | | | | | | | | | | 3085 | 5090 | 6550 |
| | | | | | | | | | | | | | | 40.0 | 48.0 | 56.3 |
| Female | HM | 9704-0501 | | | | | | | | | | | | 3194 | 4700 | 5880 |
| | | | | | | | | | | | | | | 40.0 | 48.1 | 57.4 |
| Female | HM | 9705-0501 | | | | | | | | | | | | 3120 | 4500 | 5702 |
| | | | | | | | | | | | | | | 40.0 | 48.1 | 57.1 |
| Female | HM | 9705-0502 | | | | | | | | | | | | 4080 | 6327 | 7348 |
| | | | | | | | | | | | | | | 40.0 | 48.3 | 57.3 |
| Female | HM | 9706-0501 | | | | | | | | | | | | 3396 | 5000 | 6645 |
| | | | | | | | | | | | | | | 40.0 | 48.3 | 58.1 |
| Female | HM | 9706-0502 | | | | | | | | | | | | 3041 | 4315 | 5525 |
| | | | | | | | | | | | | | | 40.0 | 47.7 | 57.6 |
| Female | HM | 9707-0501 | | | | | | | | | | | | 4653 | 5515 | 6770 |
| | | | | | | | | | | | | | | 40.0 | 47.9 | 56.6 |
| Female | HM | 9707-0502 | | | | | | | | | | | | 3419 | 5500 | 7080 |
| | | | | | | | | | | | | | | 40.0 | 48.0 | 57.1 |
| Female | HM | 9707-0503 | | | | | | | | | | | | 3773 | 5785 | 7675 |
| | | | | | | | | | | | | | | 40.0 | 47.9 | 56.9 |
| Female | HM | 9707-0505 | | | | | | | | | | | | 3716 | | |
| | | | | | | | | | | | | | | 40.0 | | |
| Female | HM | 9708-0501 | | | | | | | | | | | | 3688 | 5440 | 6890 |
| | | | | | | | | | | | | | | 40.0 | 48.1 | 57.6 |
| Female | HM | 9708-0502 | | | | | | | | | | | | 3454 | 5192 | 5950 |
| | | | | | | | | | | | | | | 40.0 | 48.1 | 57.4 |
| Female | HM | 9708-0503 | | | | | | | | | | | | 2977 | 5165 | 7040 |
| | | | | | | | | | | | | | | 40.0 | 48.1 | 57.4 |
| Female | HM | 9708-0504 | | | | | | | | | | | | 3864 | 5660 | 6705 |
| | | | | | | | | | | | | | | 40.0 | 48.4 | 57.4 |
| Female | HM | 9708-0505 | | | | | | | | | | | | 3831 | 5800 | 7435 |
| | | | | | | | | | | | | | | 40.0 | 47.7 | 57.6 |
| Female | HM | 9709-0501 | | | | | | | | | | | | 3550 | | |
| | | | | | | | | | | | | | | 40.0 | | |
| Female | HM | 9709-0502 | | | | | | | | | | | | 3715 | 5205 | 6100 |
| | | | | | | | | | | | | | | 40.0 | 48.0 | 56.9 |
| Female | HM | 9709-0503 | | | | | | | | | | | | 3195 | | |
| | | | | | | | | | | | | | | 40.0 | | |
| Female | HM | 9709-0504 | | | | | | | | | | | | 3190 | 4590 | |
| | | | | | | | | | | | | | | 40.0 | 48.3 | |
| Female | HM | 9709-0506 | | | | | | | | | | | | 3505 | 4500 | 5910 |
| | | | | | | | | | | | | | | 40.0 | 48.0 | 57.1 |

| Gender | Regimen | SUBJECT | Variable | Wgt1 | Wgt2 | Wgt3 | Wgt4 | Wgt5 | Wgt6 | Wgt7 |
|---|---|---|---|---|---|---|---|---|---|---|
| Male | Control | 9712-0301 | Weight (g) | 1245 | 1221 | 1245 | 1291 | 1294 | 1330 | 1369 |
| | | | Age (weeks pca) | 31.6 | 31.7 | 31.9 | 32.0 | 32.1 | 32.3 | 32.4 |
| Male | DHA | 9707-0307 | Weight (g) | 1649 | 1675 | 1699 | 1732 | 1778 | 1811 | 1858 |
| | | | Age (weeks pca) | 32.4 | 32.6 | 32.7 | 32.9 | 33.0 | 33.1 | 33.3 |
| Female | Control | 9698-0003 | Weight (g) | 1020 | 1050 | 1070 | 1080 | 1080 | 1060 | 1080 |
| | | | Age (weeks pca) | 31.1 | 31.3 | 31.4 | 31.6 | 31.7 | 31.9 | 32.0 |
| Female | DHA + ARA | 9705-0005 | Weight (g) | 1075 | 1120 | 1185 | 1280 | 1310 | 1310 | 1265 |
| | | | Age (weeks pca) | 31.1 | 31.4 | 31.7 | 32.1 | 32.4 | 32.7 | 33.0 |

| | | | | | | | | | | | | Growth |
|---|---|---|---|---|---|---|---|---|---|---|---|---|
| | | | | | | | | | | | | Rate |
| Gender | Wgt8 | Wgt9 | Wgt10 | Wgt11 | Wgt12 | Wgt13 | Wgt14 | Wgt15 | Wgt16 | Wgt17 | Wgt18 | g/day |
| Male | 1402 | 1433 | 1448 | 1465 | | | | | | | | 26.1 |
| | 32.6 | 32.7 | 32.9 | 33.0 | | | | | | | | |
| Male | 1882 | 1938 | 1994 | 2030 | 2075 | | | | | | | 39.6 |
| | 33.4 | 33.6 | 33.7 | 33.9 | 34.0 | | | | | | | |
| Female | 1070 | | | | | | | | | | | 5.6 |
| | 32.1 | | | | | | | | | | | |
| Female | 1350 | 1380 | 1440 | 1450 | 1510 | 1515 | 1565 | 1585 | 1640 | 1680 | 1670 | 22.1 |
| 33.3 | 33.4 | 33.7 | 33.9 | 34.0 | 34.1 | 34.3 | 34.4 | 34.6 | 34.7 | 34.9 | | |

*Four subjects had more the 9 weights used in growth rate calculation. A complete listing appears on the last page.

What is claimed is:

1. A method for providing nutrition to infants comprising:
administering to the infants an infant nutritional product or infant nutritional supplement having DHA in an amount of at least 10 mg per 100 kcal nutritional product or supplement and having ARA in an amount of at least about 30 mg per 100 kcal nutritional product or supplement, wherein the DHA and ARA are derived from a source that is selected from the group consisting of fish oils, single cell oils and brain lipids.

2. The method according to claim 1, wherein the nutritional product or supplement is an infant formula.

3. The method according to claim 2, wherein the infant formula is nutritionally complete and comprises lipid in an amount of from about 3 g/100 kcal to about 7 g/100 kcal, protein in an amount of from about 1 g/100 kcal to about 5 g/100 kcal, and carbohydrate in an amount of from about 8 g/100 kcal to about 12 g/100 kcal.

4. The method according to claim 1, wherein the infant nutritional product or infant nutritional supplement comprises DHA in an amount of at least about 17 mg per 100 kcal nutritional product or supplement and ARA in an amount of at least about 31 mg per 100 kcal nutritional product or supplement.

5. The method according to claim 1, wherein the infant nutritional product or infant nutritional supplement comprises DHA in an amount of at least about 18.5 mg per 100 kcal nutritional product or supplement and ARA in an amount of at least about 33.6 mg per 100 kcal nutritional product or supplement.

6. The method according to claim 1, wherein the infant nutritional product or infant nutritional supplement comprises DHA in an amount of from 10 mg to about 50 mg per 100 kcal nutritional product or supplement and ARA in an amount of from about 30 mg to about 100 mg per 100 kcal nutritional product or supplement.

7. The method according to claim 1, wherein the infant nutritional product or infant nutritional supplement comprises DHA in an amount of from 10 mg to about 33 mg per 100 kcal nutritional product or supplement and ARA in an amount of from about 30 mg to about 67 mg per 100 kcal nutritional product or supplement.

8. The method according to claim 1, wherein the infant nutritional product or infant nutritional supplement comprises DHA in an amount of from 10 mg to about 25 mg per 100 kcal nutritional product or supplement and ARA in an amount of from about 30 mg to about 50 mg per 100 kcal nutritional product or supplement.

9. The method according to claim 1, wherein the infant nutritional product or infant nutritional supplement comprises DHA in an amount of from 10 mg to about 20 mg per 100 kcal nutritional product or supplement and ARA in an amount of from about 30 mg to about 40 mg per 100 kcal nutritional product or supplement.

10. The method according to claim 1, wherein the infant nutritional product or infant nutritional supplement comprises DHA in an amount of from about 15 mg to about 20 mg per 100 kcal nutritional product or supplement and ARA in an amount of from about 30 mg to about 40 mg per 100 kcal nutritional product or supplement.

11. The method according to claim 1, wherein the infant nutritional product or infant nutritional supplement comprises DHA in an amount of from about 17 mg to about 50 mg per 100 kcal nutritional product or supplement and ARA in an amount of from about 31 mg to about 100 mg per 100 kcal nutritional product or supplement.

12. The method according to claim 1, wherein the infant nutritional product or infant nutritional supplement comprises DHA in an amount of from about 17 mg to about 33 mg per 100 kcal nutritional product or supplement and ARA in an amount of from about 31 mg to about 67 mg per 100 kcal nutritional product or supplement.

13. The method according to claim 1, wherein the infant nutritional product or infant nutritional supplement comprises DHA in an amount of from about 17 mg to about 25 mg per 100 kcal nutritional product or supplement and ARA in an amount of from about 31 mg to about 50 mg per 100 kcal nutritional product or supplement.

14. The method according to claim 1, wherein the infant nutritional product or infant nutritional supplement comprises DHA in an amount of from about 17 mg to about 20 mg per 100 kcal nutritional product or supplement and ARA in an amount of from about 31 mg to about 40 mg per 100 kcal nutritional product or supplement.

15. The method according to claim 1, wherein the infant nutritional product or infant nutritional supplement comprises DHA in an amount of from about 18.5 mg to about 33 mg per 100 kcal nutritional product or supplement and ARA in an amount of from about 33.6 mg to about 67 mg per 100 kcal nutritional product or supplement.

16. The method according to claim 1, wherein the infant nutritional product or infant nutritional supplement comprises DHA in an amount of from about 18.5 mg to about 25 mg per 100 kcal nutritional product or supplement and ARA in an amount of from about 33.6 mg to about 50 mg per 100 kcal nutritional product or supplement.

17. The method according to claim 1, wherein the infant nutritional product or infant nutritional supplement comprises DHA in an amount of from about 18.5 mg to about 20 mg per 100 kcal nutritional product or supplement and ARA in an amount of from about 33.6 mg to about 40 mg per 100 kcal nutritional product or supplement.

18. The method according to claim 1, wherein the DHA and ARA are derived from a source comprising fish oils.

19. The method according to claim 1, wherein the DHA and ARA are derived from a source comprising single cell oils.

20. The method according to claim 1, wherein the DHA and ARA are derived from a source comprising brain lipids.

21. The method according to claim 1, wherein the nutritional supplements are selected from the group consisting of oil drops, sachets, and vitamins.

22. An infant formula comprising DHA in an amount of at least 10 mg per 100 kcal of infant formula and ARA in an amount of at least about 30 mg per 100 kcal of infant formula, wherein the DHA and ARA are derived from a source that is selected from the group consisting of fish oils, single cell oils and brain lipids.

23. The infant formula according to claim 22, wherein the infant formula is nutritionally complete and comprises lipid in an amount of from about 3 g/100 kcal to about 7 g/100 kcal, protein in an amount of from about 1 g/100 kcal to about 5 g/00 kcal, and carbohydrate in an amount of from about 8 g/100 kcal to about 12 g/100 kcal.

24. The infant formula according to claim 22, wherein the infant formula comprises DHA in an amount of at least about 17 mg per 100 kcal infant formula and ARA in an amount of at least about 31 mg per 100 kcal infant formula.

25. The infant formula according to claim 22, wherein the infant formula comprises DHA in an amount of at least about 18.5 mg per 100 kcal infant formula and ARA in an amount of at least about 33.6 mg per 100 kcal infant formula.

26. The infant formula according to claim 22, wherein the infant formula comprises DHA in an amount of from 10 mg to about 50 mg per 100 kcal infant formula and ARA in an amount of from about 30 mg to about 100 mg per 100 kcal infant formula.

27. The infant formula according to claim 22, wherein the infant formula comprises DHA in an amount of from 10 mg to about 33 mg per 100 kcal infant formula and ARA in an amount of from about 30 mg to about 67 mg per 100 kcal infant formula.

28. The infant formula according to claim 22, wherein the infant formula comprises DHA in an amount of from 10 mg to about 25 mg per 100 kcal infant formula and ARA in an amount of from about 30 mg to about 50 mg per 100 kcal infant formula.

29. The infant formula according to claim 22, wherein the infant formula comprises DHA in an amount of from 10 mg to about 20 mg per 100 kcal infant formula and ARA in an amount of from about 30 mg to about 40 mg per 100 kcal infant formula.

30. The infant formula according to claim 22, wherein the infant formula comprises DHA in an amount of from about 15 mg to about 20 mg per 100 kcal infant formula and ARA in an amount of from about 30 mg to about 40 mg per 100 kcal infant formula.

31. The infant formula according to claim 22, wherein the infant formula comprises DHA in an amount of from about 17 mg to about 50 mg per 100 kcal infant formula and ARA in an amount of from about 31 mg to about 100 mg per 100 kcal infant formula.

32. The infant formula according to claim 22, wherein the infant formula comprises DHA in an amount of from about 17 mg to about 33 mg per 100 kcal infant formula and ARA in an amount of from about 31 mg to about 67 mg per 100 kcal infant formula.

33. The infant formula according to claim 22, wherein the infant formula comprises DHA in an amount of from about 17 mg to about 25 mg per 100 kcal infant formula and ARA in an amount of from about 31 mg to about 50 mg per 100 kcal infant formula.

34. The infant formula according to claim 22, wherein the infant formula comprises DHA in an amount of from about 17 mg to about 20 mg per 100 kcal infant formula and ARA in an amount of from about 31 mg to about 40 mg per 100 kcal infant formula.

35. The infant formula according to claim 22, wherein the infant formula comprises DHA in an amount of from about 18.5 mg to about 33 mg per 100 kcal infant formula and ARA in an amount of from about 33.6 mg to about 67 mg per 100 kcal infant formula.

36. The infant formula according to claim 22, wherein the infant formula comprises DHA in an amount of from about 18.5 mg to about 25 mg per 100 kcal infant formula and ARA in an amount of from about 33.6 mg to about 50 mg per 100 kcal infant formula.

37. The infant formula according to claim 22, wherein the infant formula comprises DHA in an amount of from about 18.5 mg to about 20 mg per 100 kcal infant formula and ARA in an amount of from about 33.6 mg to about 40 mg per 100 kcal infant formula.

38. The infant formula according to claim 22, wherein the DHA and ARA are derived from a source comprising fish oils.

39. The infant formula according to claim 22, wherein the DHA and ARA are derived from a source comprising single cell oils.

40. The infant formula according to claim 22, wherein the DHA and ARA are derived from a source comprising brain lipids.
